(19) United States
(12) Patent Application Publication
(10) Pub. No.: US 2012/0283125 A1
(43) Pub. Date: Nov. 8, 2012

(54) Ovarian Markers of Oocyte Competency and Uses Thereof

(75) Inventors: Marc-André Sirard, Quebec (CA); Mourad Assidi, Montreal (CA); Mélanie Hamel, Saint-Augustin-de-Desmaures (CA); Gilles Hamel, Donnaconna (CA); Claude Robert, St-Nicolas (CA); Hana Kovarova, Pardubice (CZ)

(73) Assignee: UNIVERSITÉ LAVAL, Québec, QC (CA)

(21) Appl. No.: 13/509,379

(22) PCT Filed: Nov. 12, 2010
(86) PCT No.: PCT/CA10/01813

§ 371 Date: Jul. 23, 2012

Related U.S. Application Data
(60) Provisional application No. 61/260,599, filed on Nov. 12, 2009.

(51) Int. Cl. C40B 30/04; G01N 21/76; C12Q 1/68

(52) U.S. Cl. 506/9; 435/6.13; 435/7.9; 435/6.12

(57) ABSTRACT

The present invention relates to the competence of oocytes to fertilization, uterine implantation and development into a living being. The invention describes ovarian markers whose expression is predicative of oocyte competency that are detected and/or measured in follicular fluid, cumulus cells and/or follicular cells of a mammal. Also described are methods for evaluating competence of mammalian oocytes, methods for selecting a mammalian oocyte for assisted reproduction (AR), and screening methods for identifying stimulatory or inhibitory compounds to mammalian oocyte competence.

### RELATED APPLICATION

This application claims priority to U.S. provisional application No. 61/260,599 filed on Nov. 12, 2009 which is incorporated herein by reference in its entirety.

### FIELD OF THE INVENTION

The present invention relates to field of fertility. More particularly, it relates to follicular fluid, follicular cells and cumulus cells markers of mammalian oocyte competency and uses thereof.

### BACKGROUND OF THE INVENTION

Oocyte's quality largely depends on the follicle from which it originates, as shown in a number of animal and human studies. During the IVF procedure upon ovarian stimulation and ovulation induction, a cohort of heterogeneous follicles is recruited to develop and ovulate, irrespective of their differentiate state. This creates an asynchrony in the maturation process and heterogeneity in the quality of the oocytes recovered for assisted reproduction. To determine the factors associated with the developmental competence of the oocytes and to understand how they positively influence the oocyte quality, follicles with different oocyte quality must be analyzed for these factors at the protein and gene levels.

Previous studies have tended to focus upon the appearance of the embryo (morphology) to predict the success of fertilization in vitro. Other means of investigate the embryo quality may interfere with embryo viability leading to an absence of objective criteria to distinguish between several embryos, which to transfer to the mother. In recent years, scientific evidences obtained both from animal models and humans are supporting the hypothesis that the oocyte quality and therefore its ability to implant post transfer depends on the follicular conditions prevailing in the ovary before the oocytes are removed. This leads to a method of predicting the outcome of IVF which involved firstly determining the level of target compounds in a biological sample taken from a female patient and then predicting, from the level of the compounds determined, the probability of establishing pregnancy in the subject by IVF. The activity measured for a pool of cells from different follicles (from the same individual) was not always a true reflection of activity in individual follicles, suggesting that one or more follicles possess compounds affecting the probability of establishing a pregnancy.

A major problem in identifying which oocytes are competent to become embryos is the fact that any procedure designed for such purpose must not adversely affect the quality or viability of the oocytes.

International PCT Patent publications WO 2007/130673 and WO 2008/066655 describe a series of oocyte, follicular fluid, and/or cumulus cells markers for evaluating the competence of a mammalian oocyte. International PCT Patent publication no. WO 2008/031226 describes using granulosa markers for determining oocyte competence. Scientific publications by the inventors also describe marker genes as pregnancy predictors (Hamel et al., (2010) Mol. Hum. Reprod., Vol. 16, No. 8, pp. 548-556; Assidi M, Montag M, Van Der Ven K, Sirard M A. Biomarkers of human oocyte developmental competence expressed in cumulus cells before ICSI: a preliminarystudy. J Assist Reprod Genet. 2010 Oct. 16). Considering the state of the art, there is still a need for biological markers and noninvasive characterization methods for determining the competency of oocytes.

### SUMMARY OF THE INVENTION

The present invention contemplates the use of follicular fluid, follicular cells and cumulus cells markers for evaluating the competence of mammalian oocytes for numerous assisted reproduction techniques, for implantation and pregnancy induction or both. As used herein the term “assisted reproduction” or “AR” broadly refers to methods, procedures and techniques wherein oocytes and/or embryos are manipulated, including, but not limited to, in vitro fertilization (IVF), artificial insemination (Al), intracytoplasmic sperm injection (ICSI), zygote intrafallopian transfer (ZIFT), pronuclear stage tubal transfer (PROST), and embryo transfer.

On aspect of the invention concerns a method for evaluating competence of a mammalian oocyte comprising assessing expression of at least one ovarian marker from an ovarian follicle comprising said oocyte. The oocyte may be from a human oocyte. The oocyte and the ovarian marker may be from a single follicle. The polynucleotide may be a DNA or a RNA sequence. The ovarian marker is selected from the genes listed in Tables 2A, 2B, 4 to 8 and 10, and combinations thereof. Particular embodiments comprises assessing expression of at least 3, 3, 5 or more markers.

In accordance with another embodiment the ovarian marker is a follicular cell marker which is expressed in follicular cells comprised in the ovarian follicle. Preferred follicular cell markers include UGP2, PHLDA1, GAPBPI, SFRP1, HOMER1, LRP8, DPYSL3, PGR, YWHAZ, MARCKS, SEMA3A, PIR, EREG and combinations thereof.

In accordance with one embodiment the ovarian marker is a cumulus cell marker which is expressed in cumulus cells originating form the oocyte e.g. surrounding the oocyte in the ovarian follicle. Preferred cumulus cell markers include the genes listed in Tables 4 to 8 and combinations thereof.

In accordance with one embodiment the ovarian marker is a follicular fluid marker which is present in follicular fluid comprised in the ovarian follicle. Preferred follicular fluid markers include Ceruloplasmin precursor, Apolipoprotein A-IV precursor, β-actin (ACTB) and combinations thereof. Follicular fluid may be obtained before ovulation by aspirating the ovarian follicle before ovulation.

In preferred embodiments, the methods of the invention comprises comparing the expression level of the at least one marker with a control expression level. Assessment of the expression of the marker may comprises measuring polynucleotide and/or polypeptide expression levels for the marker. Examples of polynucleotides and polypeptide to be measured includes sequence as set forth in GenBank™ or Unigene™ for the accession numbers provided in Tables 2A, 2B, 4 to 8 and 10.

Another aspect of the invention concerns a method for evaluating competence of a mammalian oocyte, the method comprising assessing expression of at least one follicular cell marker which is expressed in follicular cells of an ovarian follicle comprising the mammalian oocyte, the expression level of the follicular cell marker being predicative of oocyte competency. The follicular cell marker is selected from UGP2, PHLDA1, GAPBP1, SFRP1, HOMER1, LRP8, DPYSL3, PGR, YWHAZ, MARCKS, SEMA3A, PIR, EREG and combinations thereof. Assessment of the expression of the at least one follicular cell marker may comprises measuring polynucleotide (e.g. DNA and/or RNA levels) and/or polypeptide expression levels for said cumulus cell marker(s).

In one particular embodiment, the method of evaluating competence comprises:
(a) assessing in follicular cells originating from an ovarian follicle comprising the oocyte an expression level of at least one polynucleotide, wherein the at least one polynucleotide comprises a nucleotide sequence as set forth in GenBank™ or Unigene™ for the accession numbers provided in Tables 2A and 2B; and
(b) comparing the expression level of the at least one polynucleotide with a control expression level;
   wherein a differential between expression level of the at least one polynucleotide and the control expression level is predicative of oocyte competency.

In another particular embodiment, the method of evaluating competence comprises:
(a) assessing in follicular cells originating from an ovarian follicle comprising the oocyte an expression level of at least one polypeptide, wherein said polypeptide comprises an amino acid sequence as set forth in GenBank™ or Unigene™ for the accession numbers provided in Tables 2A and combinations thereof; and
(b) comparing the expression level of the at least one polypeptide with a control expression level;
   wherein a differential between expression level of the at least one polypeptide and the control expression level is predicative of oocyte competency.

Another aspect of the invention concerns a method for evaluating competence of a mammalian oocyte, the method comprising assessing expression of at least one cumulus cell marker which is expressed in cumulus cells originating form the oocyte e.g. surrounding the oocyte in the ovarian follicle, the expression level of the cumulus cell marker being predicative of oocyte competency. The cumulus cell marker is selected from the genes listed in Tables 4 to 8 and combinations thereof. Assessment of the expression of the at least one cumulus cell marker may comprises measuring polynucleotide (e.g. DNA and/or RNA levels) and/or polypeptide expression levels for said cumulus cell marker(s).

According to a particular embodiment, the method of evaluating competence of a mammalian oocyte comprises:
(a) assessing in cumulus cells originating from the oocyte an expression level of at least one polynucleotide, wherein the polynucleotide comprises a nucleotide sequence comprising any one of SEQ ID NOs: 88 to 109 or comprising a sequence as set forth in GenBank™ or Unigene™ for the accession numbers provided in Tables 4 to 8; and
(b) comparing the expression level of the at least one nucleotide with a control expression level;
   wherein a differential between expression level of the at least one nucleotide and the control expression level is predicative of oocyte competency.

According to another particular embodiment, the method of evaluating competence of a mammalian oocyte comprises:
(a) assessing in cumulus cells originating from the oocyte an expression level of at least one polypeptide, wherein the polypeptide comprises an amino acid sequence encoded by a nucleotide comprising any one of SEQ ID NOs: 88 to 109 or an amino acid sequence as set forth in GenBank™ or Unigene™ for the accession numbers provided in Tables 4 to 8;
and (b) comparing the expression level of the at least one polypeptide with a control expression level;
   wherein a differential between expression level of the at least one polypeptide and the control expression level is predicative of oocyte competency.

Another aspect of the invention concerns a method for evaluating competence of a mammalian oocyte, the method comprising assessing expression of at least one follicular fluid marker which is present in follicular fluid from an ovarian follicle comprising the mammalian oocyte, the expression level of the follicular fluid marker being predicative of oocyte competency. The follicular fluid marker is a protein selected from Ceruloplasmin precursor, Apolipoprotein A-IV precursor, β-actin (ACTB) and combinations thereof. Assessment of the presence of the at least one follicular fluid marker typically comprises measuring polypeptide expression levels, but it may under some particular circumstances comprises measuring polynucleotides (e.g. DNA and/or RNA levels).

According to a particular embodiment, the method of evaluating competence of a mammalian oocyte comprises:
(a) assessing in follicular fluid originating from an ovarian follicle comprising the oocyte an expression level of at least one polypeptide, wherein the polypeptide is selected from the group consisting of Ceruloplasmin precursor, Apolipoprotein A-IV precursor, β-actin (ACTB) and combinations thereof; and
(b) comparing the expression level of the at least one polypeptide with a control expression level;
   wherein a differential between expression level of the at least one polypeptide and the control expression level is predicative of oocyte competency.

The methods of the invention may further comprises comparing the expression level with expression level of control follicular cells, cumulus cells and/or follicular fluid and showing a significant change by using ratios or absolute amount to reflect oocyte competence.

Other aspects of the invention concerns a method for selecting a mammalian oocyte for assisted reproduction (AR) and methods for screening a compound stimulatory or inhibitory to oocyte competence, uterus implantation of an embryo and/or development into living individual at birth.

According to a particular embodiment, the method for selecting a mammalian oocyte for assisted reproduction (AR) comprises:
obtaining mammalian follicular cells of an ovarian follicle which contains the oocyte;
determining expression level of at least one follicular cell marker, wherein the at least one follicular cell marker is selected from the group consisting of UGP2, PHLDA1, GAPBP1, SFRP1, HOMER1, LRP8, DPYSL3, PGR, YWHAZ, MARCKS, SEMA3A, PIR, EREG and combinations thereof;
comparing the expression level of the at least one marker with a control expression level in control follicular cells; and
selecting for AR an oocyte which follicular cells have a desirable expression level of the at least one marker when compared with the control expression level.

According to another particular embodiment, the method for selecting a mammalian oocyte for assisted reproduction (AR) comprises:
obtaining mammalian cumulus cells originating from the oocyte;
determining expression level of at least one cumulus cell marker, wherein the at least one cumulus cell marker is selected from the group consisting of genes listed in Tables 4 to 8 and combinations thereof;
comparing the expression level of the at least one marker with a control expression level in control cumulus cells; and
selecting for AR an oocyte which cumulus cells have a desirable expression level of the at least one marker when compared with the control expression level.

According to another particular embodiment, the method for selecting a mammalian oocyte for assisted reproduction (AR) comprises:
obtaining mammalian follicular fluid from an ovarian follicle which contains the oocyte;
determining in the follicular fluid expression level of at least one follicular fluid marker, wherein the follicular fluid marker is a protein selected from the group consisting of Ceruloplasmin precursor, Apolipoprotein A-IV precursor, β-actin (ACTB) and combinations thereof;
comparing the expression level of the at least one marker with a control expression level in control follicular fluid; and
selecting for AR an oocyte which follicular fluid have a desirable expression level of said at least one marker when compared with the control expression level.

Also provided is a kit for use in evaluating competence of mammalian oocytes. An array of nucleic acid probes immobilized on a solid support is also described.

An advantage of the invention is that it provides predictive tools for determining in advance the competency of an oocyte for assisted reproduction (AR), to embryo viability, to embryo development and/or to embryo implantation. The invention also provides non-invasive and non-damaging methods for selecting embryos to be transferred to a recipient, thereby reducing the need of multiple embryo transfer while maximizing pregnancy rates. The markers of the invention may also serve as indicators of successful ovarian hormonal stimulation regimen which can be a useful diagnostic tool to refine hormonal treatment of a patient or a population of patients. In addition, the markers of the invention may be helpful in optimizing in vitro maturation (IVM) media, both in terms of type and levels of components. Examples of possible applications can be found in the scientific literature, for instance in Hamel et. al., 2010 (Mol. Hum. Reprod., Vol. 16, No. 8, pp. 548-556) and Albuz et al., Simulated physiological oocyte maturation (SPOM): a novel in vitro maturation system that substantially improves embryo yield and pregnancy outcomes. Hum Reprod. 2010 Sep. 24).

Additional aspects, advantages and features of the present invention will become more fully understood from the detailed description given herein and from the accompanying drawings, which are exemplary and should not be interpreted as limiting the scope of the invention.

### BRIEF DESCRIPTION OF THE FIGURES

FIG. 1 are bar graphs showing relative mRNA levels of gene markers in follicular cells associated with competent oocytes. Quantification of mRNA level by Q-PCR that showed differential expression (P<0.05) in follicular cells from oocytes associated with or without a pregnancy. ** Indicates a significant difference within gene (P<0.01), * Indicates a significant difference within gene (P<0.05). Results were presented as mean±SEM and analyzed by t-test analysis.

FIG. 2 are bar graphs showing relative mRNA levels of gene markers in follicular cells associated with non competent oocytes. Quantification of mRNA level by Q-PCR that showed a tendency (P<0.1) or similar expression (P>0.1) in follicular cells from oocytes associated with or without a pregnancy. Results were presented as mean±SEM and analyzed by t-test analysis.

FIG. 3 is a scheme of an experimental protocol used in Example 2 for comparing cumulus cells on micro-array in order to identify markers associated with the outcome (i.e. pregnancy).

FIGS. 4A and 4B are bar graphs showing differentially expressed candidates in human cumulus cells of pregnant (ZGP) versus non pregnant (ZGNP) ZG (zona good) patients as revealed by QPCR. Value (y axis) are arbitrary units following a normalization with Genorm™ and log base 10 transformation. FIG. 4A shows overexpressed candidates and FIG. 4B downexpressed candidates (α=0.05).

FIG. 5 are pictures of electrophoretic gels illustrating protein candidates differently expressed between the pool of follicles from pregnant (Pool A) vs unpregnant (Pool B) patients.

FIG. 6 is a picture of a Western gel analysis of six (6) different pools (pregnant patient (Pool A); unpregnant (Pool B)) for one of the candidate ApoA4. The numbers above the gel represent sub-pool identification as divided in 3 replicates.

FIG. 7 is a bar graph showing a densitometric analysis of the bands shown in FIG. 6.

### DETAILED DESCRIPTION OF PREFERRED EMBODIMENTS

The present invention provides, by the analysis of marker expression, non-damaging and noninvasive methods of distinguishing and characterizing oocytes and embryos more likely to experience successful fertilization and implantation from oocytes and embryos less likely to experience successful fertilization and implantation.

The invention identifies biological ovarian markers from the follicular fluid, the cumulus cells and follicular cells which are predictive of oocyte competency in mammals. For instance, the markers of the invention may be used to assess quality of an oocyte for fertilization and subsequent embryo quality (e.g. viability, likelihood of successful implantation, resistance to long-term storage and freezing, etc).

### Definitions

For the purpose of the present invention the following terms are defined below.

As used herein and in the appended claims, the singular forms “a”, “an”, and “the” include plural referents unless the context clearly indicates otherwise. Thus, for example, reference to “a marker” includes one or more of such markers and reference to “the method” includes reference to equivalent steps and methods known to those of ordinary skill in the art that could be modified or substituted for the methods described herein.

The term “subject” includes living organisms in which evaluation of oocyte competence is desirable. The term “subject” includes female animals (e.g., mammals (e.g., cats, dogs, horses, pigs, cows, goats, sheep, rodents (e.g., mice or rats), rabbits, squirrels, bears, primates (e.g., chimpanzees, monkeys, gorillas, and humans)), as well as avians (e.g. chickens, ducks, Peking ducks, geese), and transgenic species thereof. Preferably, the subject is a mammal. More preferably, the subject is a human. Even more preferably, the subject is a human patient in need of or receiving in vitro fertilization treatment.

The term “competence” as used herein is intended to mean the competence, or competency, both terms being equivalent, of an oocyte for fertilization, implantation and development into living individual.

The term “ovarian marker” as used herein refers to particular genes expressed in ovarian follicles which expression is predictive of the competence of the oocytes comprised in those follicles.

The term “follicular fluid” is the liquid which fills the follicular antrum and surrounds the ovum (oocyte) in an ovarian follicle.

The term “cumulus cells” refers to cells which originates from or are connected to (e.g. surrounding and nourishing) the oocyte in an ovarian follicle. This cluster of cells is also termed the cumulus oophorus.

The term “follicular cells” as used herein defines the cells that are obtained by follicular aspiration at the time of oocyte collection, these cells consisting essentially of granulosa cells. When the antrum develops and enlarges, the follicular cells divide into two functional groups: the cells in immediate contact with the oocyte which are called the cumulus cells (cumulus oophorus) and the mural granulosa cells which line the follicular wall around the follicular antrum. Cumulus cells express characteristics distinct from the mural granulosa cells. Those skilled in the art are aware that by aspirating follicular content near ovulation often result in a mix of cumulus and granulosa cells, and may be some blood. Since the cumulus cells are removed with the oocyte, the follicular cells remaining for the analysis are mainly granulosa cells.

An “oligonucleotide” or “polynucleotide” is a nucleic acid molecule ranging from at least 2, preferably at least 8, 15 or 25 nucleotides in length, but may be up to 50, 100, 1000, or 5000 nucleotides long or a compound that specifically hybridizes to a polynucleotide. Polynucleotides include DNA and fragments thereof, RNA and fragments thereof, cDNAs and fragments thereof, expressed sequence tags, artificial sequences including randomized artificial sequences.

As used herein, the term “polypeptide” or “protein” refers to any amino acid sequence derived from the expression of a nucleic acid sequence or gene encoding an ovarian marker as defined herein. The term is intended to encompass complete proteins and fragments thereof.

### Evaluation of Oocyte Competence

Evaluation of oocyte quality and competency may serves different uses. For instance, in one embodiment evaluation of oocyte competence is carried out to predict the outcome of assisted reproduction (AR) techniques (e.g. in vitro fertilization (IVF), artificial insemination (AI), intracytoplasmic sperm injection (ICSI), zygote intrafallopian transfer (ZIFT), pronuclear stage tubal transfer (PROST), and embryo transfer) and embryo implantation in a female individual. More particularly, the markers according to the invention are useful for determining the competence of fertilized oocytes and embryos, to implant (or, more accurately, successfully implant) in the uterus of a recipient female, and to develop into a living being. Accordingly, the markers and methods of the invention are useful to perform the screening of competent embryos before their transfer in a recipient human or animal female. Yet, the follicular fluid, the cumulus cells and/or granulosa cells markers may be used for evaluating whether a female subject is fertile or infertile.

In one embodiment evaluation is performed before fertilization, to assist in maximizing the generation of chromosomally normal embryos or to assist in minimizing the generation of chromosomally abnormal embryos. Yet, in another embodiment, the follicular fluid, the cumulus cells and/or follicular cells markers are used to assess whether an oocyte is chromosomally normal (e.g. in vitro assessment of oocyte aneuploidy).

In another embodiment it is performed before implantation to assist in maximizing the implantation of chromosomally normal embryos or to assist in minimizing the implantation of chromosomally abnormal embryos (e.g. diagnose chromosome abnormality).

The markers of the invention may be used to assess and/or to optimize methods for ovarian stimulation and/or for modifying or optimizing an in vitro maturation medium (e.g. identity and/or levels of components). The assessment of marker expression in follicular fluid, cumulus cells and/or follicular cells according to the invention may also be useful to assist the proper function of affected gene expression pathways for example, assay the effects of toxicants on human oocytes and/or human embryos. Accordingly, a related aspect of the invention concerns methods of diagnosis wherein levels of expression of the biological markers of the invention are used to determine the outcome of the assisted reproduction procedures. Another related aspect concerns methods wherein assessment of the expression of the biological markers of the invention are used to determine the suitability of a female individual for assisted reproduction treatment, and/or for optimizing for ovarian stimulation protocols.

One particular aspect of the invention concerns an in vivo method for assessing a compound stimulatory or inhibitory activity to oocyte competence in a subject, the method comprising the steps of:
a) administering to the subject a candidate compound which activity to stimulate or inhibit oocyte competency is to be assessed;
b) obtaining from the subject follicular fluid, follicular cells and/or cumulus cells from an ovarian follicle comprising the oocyte; and
c) determining in follicular fluid, follicular cells and/or cumulus cells the expression level of at least ovarian marker selected among the genes listed in Tables 2A, 2B, 4 to 8 and 10, and combinations thereof, wherein the expression level is predicative of oocyte competency.

In a related embodiment, the method further comprises step d) of comparing the expression level measured in step c) with the expression level of follicular fluid, follicular cells and/or cumulus cells from a subject not exposed to the candidate compound and differences in the expression levels is indicative of the compound stimulatory or inhibitory effect.

In practice, evaluating competence of a mammalian oocyte is carried out by assessing expression of one or the biological marker(s) according to the invention from the same follicle from which are sampled the follicular fluid, cumulus cell(s) and/or the follicular cell(s). In preferred embodiments, the subject's follicular fluid, follicular cells and/or cumulus cells are obtained is(are) obtained before ovulation by aspirating an ovarian follicle comprising said oocyte.

The oocyte can be obtained at a desired stage by in vivo or in vitro maturation, and the embryo can be produced by in vitro fertilization or sperm nuclear transfer into the oocyte(s). Preferably, the oocytes, follicular fluid, cumulus cells and/or follicular cells and embryos are human. However, the oocytes, follicular fluid, cumulus cells and/or follicular cells and embryos may be obtained from other non-human animals, for instance domesticated animals.

Quantity of fluid or number of cells (one or more) to be used for assessing expression levels will vary according to various factors, including but not limited to the particular marker being assessed, the source and quality of the sample, the measurement technique being used, the subject's condition, the collection protocol in the clinic, etc.

According to the present invention, oocytes, follicular fluid, cumulus cells and follicular cells can be harvested by methods and techniques known in the art, including direct aspiration of the ovarian follicle a subject's with an appropriate needle via the subject's vagina or any other route. Is some embodiment, oocytes, follicular fluid, cumulus cells and follicular cells may be obtained by puncture of an ovarian follicle from an ovary outside the patient's body. Typically the time of collection of the oocyte defines if the oocyte requires in vitro maturation (in vitro oocyte) or not (in vivo oocyte). The present invention encompasses both, in vitro and in vivo oocytes.

It is also conceivable according to the invention to assess indirectly expression of selected markers by measuring culture medium in which the oocytes, cumulus cells, or embryos are or have been cultured. Uses of metabolomic approaches are within the scope of the invention.

### Measurement Methods

The inventions contemplates using methods known to those skilled in the art for the identification of differently expressed markers and/or assessment of markers expression levels or marker expression products, such as RNA and protein, in follicular fluid, cumulus cells, and follicular cells. As used herein, the term “marker expression” or “expression of a [X] marker” encompasses the transcription, translation, post-translation modification, and phenotypic manifestation of a gene, including all aspects of the transformation of information encoded in a gene into RNA or protein. By way of non-limiting example, marker expression includes transcription into messenger RNA (mRNA), and translation into protein.

The terms “assessing expression” is meant an assessment of the degree of expression of a marker in a sample at the nucleic acid or protein level, using technology available to the skilled artisan to detect a sufficient portion of any marker expression product (including nucleic acids and proteins) of any one of the genes listed herein in Tables 2A, 2B, 4 to 8 and 10 and/or any of the sequences listed herein in the accompanying sequence listing, such that the sufficient portion of the marker expression product detected is indicative of the expression of any one of the genes listed herein in Tables 2A, 2B, 4 to 8 and 10 and/or any one of the sequences listed herein in the accompanying sequence listing.

Any suitable method known in the art can be used to measure the marker's expression. For instance, assessment of the expression of the markers according to the invention may comprise detecting and/or measuring le level of one or more marker expression products, such as mRNA and protein.

According to the invention, specific markers are selected depending of the origin of the biological materials. For instance, in one embodiment the marker is a follicular cell marker which is selected from UGP2, PHLDA1, GAPBP1, SFRP1, HOMER1, LRP8, DPYSL3, PGR, YWHAZ, MARCKS, SEMA3A, PIR, EREG and combinations thereof. In a preferred embodiment the follicular cell marker is selected from UGP2, PHLDA1, GABPB1, SFRP1, HOMER1, and combinations thereof. In some embodiments, the invention comprises assessing expression of follicular cell marker(s) by measuring levels of expression at the polynucleotide level. In some embodiments, the invention comprises assessing expression of follicular cell marker(s) by measuring levels of expression at the polypeptide level, including but not limited to measuring levels of entire proteins, polypeptides, and fragments of the polypeptides encoded by the polynucleotides. Polynucleotide and polypeptide sequences of the follicular cell marker according to the invention can easily be found by consulting the in GenBank™ or Unigene™ databases for the accession numbers provided in Tables 2A and 2B. Additional nucleotides sequences for selected follicular cell markers within the scope of the present invention are disclosed in SEQ ID NO: 13 (UGP2), SEQ ID NO: 14 (PHLDA1), SEQ ID NO: 15 (SFRP1), SEQ ID NO: 16 (HOMER1), and SEQ ID NO: 17 (GABPB1).

In another embodiment the marker is a cumulus cell marker which is selected from the genes listed in Tables 4 to 8 and combinations thereof. In a preferred embodiment the cumulus cell marker is selected from NRP1, TOM1, UBQLN1, PSMD6, DPP8, HIST1H4C, CALM1, TUG1, THOC2, SYT11, RPL9, PKN2, CAL U, CHD9, AR, SPHKAP, CHGB and combinations thereof. In another preferred embodiment the cumulus cell marker is selected from NRP1, TOM1, UBQLN1, PSMD6, DPP8, HIST1H4C, CALM1, and combinations thereof. In some embodiments, the invention comprises assessing expression of cumulus cell marker(s) by measuring levels of expression at the polynucleotide level. In some embodiments, the invention comprises assessing expression of cumulus cell marker(s) by measuring levels of expression at the polypeptide level, including but not limited to measuring levels of entire proteins, polypeptides, and fragments of the polypeptides encoded by the polynucleotides. Polynucleotide and polypeptide sequences of these genes can easily be found by consulting the in GenBank™ or Unigene™ databases for the accession numbers provided in Tables 4 to 8. Additional nucleotide sequences for selected cumulus cell markers within the scope of the present invention are disclosed in SEQ ID NO: 7 (DPP8), SEQ ID NO: 8 (HIST1H4C), SEQ ID NO: 9 (TOM1), SEQ ID NO: 10 (HIST1H4C), SEQ ID NO: 11 (UBQLN1) and SEQ ID NO: 12 (PSMD6).

Yet, in another embodiment the marker is a follicular fluid marker which is selected from Ceruloplasmin precursor, Apolipoprotein A-IV precursor, β-actin (ACTB) and combinations thereof. In some embodiments, the invention comprises assessing expression of follicular cell marker(s) by measuring levels of expression at the polypeptide level, including but not limited to measuring levels of entire proteins, polypeptides, and fragments of the polypeptides encoded by the polynucleotides. Polynucleotide and polypeptide sequences of these three genes can easily be found by consulting the in GenBank™ or Unigene™ databases for the accession numbers provided in Table 10. Additional nucleotide and amino acid sequences for selected follicular fluid markers within the scope of the present invention are disclosed in SEQ ID NO: 1 (human ceruloplasmin nucleotide sequence: Unigene™ ref. # Hs.558314, NCBI™ ref. # NM_{—}000096.3); SEQ ID NO: 2 (human ceruloplasmin protein sequence: NCBITM ref. # NP_{—}000087.1); SEQ ID NO: 3 (human beta actin (ACTB) nucleotide sequence: Unigene™ ref. # Hs.520640, NCBI™ ref. # NM_{—}001101.3); SEQ ID NO: 4 (human beta actin (ACTB) protein sequence: NCBI™ ref. # NP_{—}001092.1); SEQ ID NO: 5 (human apolipoptrotein A-IV (APOA4) nucleotide sequence: Unigene™ ref. # Hs.591940, NCBI™ ref. # NM_{—}000482.3); and SEQ ID NO: 6 (human apolipoptrotein A-IV (APOA4) protein sequence: NCBI™ ref. # NP_{—}000473.2).

Assessment of the expression of the ovarian markers described herein may comprises measuring polynucleotide levels (e.g. DNA and/or mRNA levels) and/or polypeptide expression levels for such markers. In some embodiments assessment of the marker's expression comprises measuring polynucleotide, or fragments thereof (e.g. 10, 50, 75, 100, 150, 200, 250, 300, 400, 500 or more nucleotides in length), the polynucleotide comprising a sequence as set forth in GenBank™ or Unigene™ for the accession numbers provided in Tables 2A, 2B, 4 to 8 and 10. In other embodiments assessment of the marker's expression comprises measuring a polypeptide, or a fragment thereof (e.g. 10, 15, 25, 50, or more amino acid in length), the polypeptide comprising an amino acid sequence as set forth in GenBank™ or Unigene™ for the accession numbers provided in Tables 2A, 2B, 4 to 8 and 10. Those skilled in the art will know how to select appropriate markers reported herein and identify suitable polynucleotide or polypeptide sequences providing a desired sensitivity and specificity.

In some embodiments, assessment of the follicular fluid, cumulus cells or follicular cells marker's expression is carried out by using genetic tools and related molecular biology techniques. Any conventional technique of molecular biology known to those in the art can be used, including but not limited to amplification and hybridization-related methods, and more particularly nucleic acid arrays and microarrays, PCR amplification, ligase chain reaction (LCR), polynucleotide hybridization assays (e.g. Northern blot, Southern blot, etc), deep sequencing and the like. Those skilled the art are capable of selecting suitable tools and techniques for measurement methods of gene expression.

In some embodiments, the invention contemplates the use of nucleic acid probes capable of specifically hybridizing to a mRNA of interest, and oligonucleotides or PCR primers capable of specifically amplifying a target nucleotide sequence. The nucleic acid probes, oligonucleotides or PCR primers may be of about 5 to 200 nucleic acids in length (e.g. about 5, about 10, about 15, about 20, about 25, about 30, about 50, about 75, about 100, about 125, about 150, about 175, about 200). The ways of preparing such nucleic acid probes, oligonucleotides or PCR primers are well known by persons skilled in the art. PCR analysis is preferably performed as reverse-transcriptase PCR (RT-PCR). PCR amplification products can be measured in real time for precise quantification (Real-time PCR). Tables 2A, 2B, 2C and Table 4 hereinafter provides selected examples of suitable primers according to the invention.

Hybridized nucleotides can be detected by detecting one or more labels attached to sample nucleic acids or to a probe. Labels and dyes can also be used for protein and polypeptide detection. Examples of useful labels for use in the present invention include, but is not limited to, biotin for staining with labelled streptavidin conjugate, anti-biotin antibodies, magnetic beads, fluorescent dyes (e.g. fluorescein, texas red, rhodamine, green fluorescent protein, and the like), radiolabels, phosphorescent labels, enzymes (e.g. horse radish peroxidase, alkaline phosphatase), and colorimetric labels such as colloidal gold or colored glass or plastic.

In some embodiments, assessment of the follicular fluid, cumulus cells or follicular cells marker's expression is carried out by using polypeptide-related tools and detection techniques. Any conventional technique known to those in the art can be used, including but not limited to competitive and non competitive immunoassays (e.g. sandwich assays, ELISA, RIA, chemiluminescent detection, etc.), electrophoresis and chromatography (liquid chromatography, capillary electrophoresis, quantitative western blotting, etc.), fluorescent probes, absorption matrices, mass spectrometry, and the like. Antibodies capable of specifically binding to polypeptides expressed by the gene of interest may be particularly useful. In addition, any established or newly quantitative technique known in the art can be used, alone or in combination with other techniques, in the accurate assessment of follicular fluid, cumulus cells and/or follicular cells markers expression. Those skilled the art are capable of selecting suitable tools and techniques for measurement methods of polypeptide expression levels.

The present invention may also make use of various computer program products and software for a variety of purposes, such as probe design, management of data, statistical analysis, mathematical algorithms and instrument operation. Additionally, the present invention may have include methods for providing results and genetic information over networks such as the Internet.

In another embodiment, the competence of an oocyte can be addressed by the measurement of a plurality of follicular fluid, cumulus cells and granulosa markers according to the invention. Measurement of a plurality of markers may be helpful in drawing gene expression profile pattern of a tested oocyte and in establishing a subject's expression profile. An expression profiles may be helpful in establishing more finely the competence of an oocyte as defined herein. In some embodiments, the methods of the invention comprises assessing expression of at least 2, 3, 4, 5, 6, 7, 8, 9, 10 or more follicular fluid markers. In some embodiments, the methods of the invention comprises assessing expression of at least 2, 3, 4, 5, 6, 7, 8, 9, 10 or more cumulus cell markers. In some embodiments, the methods of the invention comprises assessing expression of at least 2, 3, 4, 5, 6, 7, 8, 9, 10 or more follicular cell markers. In some embodiments, the methods of the invention comprises assessing expression of at least 2, 3, 4, 5, 6, 7, 8, 9, 10 or more markers from different source (e.g. from follicular cells, from cumulus cells and/or from follicular fluid).

According to particular embodiments, the methods of the invention comprises assessing expression a combination of at least two follicular cell markers, the combination being selected according to Table A hereinafter.

According to particular embodiments, the methods of the invention comprises assessing expression a combination of at least three follicular cell markers, the combination being selected according to Table B hereinafter.

According to particular embodiments, the methods of the invention comprises assessing expression a combination of at least two cumulus cell markers, the combination being selected according to Table C hereinafter.

According to particular embodiments, the methods of the invention comprises assessing expression a combination of at least three cumulus cell markers, the combination being selected according to Table D hereinafter.

According to particular embodiments, the methods of the invention comprises assessing expression a combination of at least two or at least three follicular cell markers, the combination being selected according to Table E hereinafter.

According to particular embodiments, the methods of the invention comprises assessing expression a combination of at least two markers from different source (e.g. follicular fluid, cumulus cell and/or follicular cells).

Similarly, the assessment of the expression of one or more follicular fluid, cumulus cells and granulosa markers according to the invention can be used in combination with any other suitable indicator of oocyte competency, with any other suitable indicator of a female subject fertility or infertility, with any other suitable indicator of an oocyte chromosomal defectiveness, etc. in a subject. Examples of possibly useful indicators include, but are not limited to, the age, body weight, general health, hormone levels (e.g. FSH, LH), the time of the menstrual cycle, and hormonal treatment used.

TABLE A
Combination of at least two follicular cell markers

| Gene | UGP2 | PHLDA1 | GAPBP1 | SFRP1 | HOMER1 | LRP8 | DPYSL3 | PGR | YWHAZ | MARCKS |
|---|---|---|---|---|---|---|---|---|---|---|
| PHLDA1 | X | | | | | | | | | |
| GAPBP1 | X | X | | | | | | | | |
| SFRP1 | X | X | X | | | | | | | |
| HOMER1 | X | X | X | X | | | | | | |
| LRP8 | X | X | X | X | X | | | | | |
| DPYSL3 | X | X | X | X | X | X | | | | |
| PGR | X | X | X | X | X | X | X | | | |
| YWHAZ | X | X | X | X | X | X | X | X | | |
| MARCKS | X | X | X | X | X | X | X | X | X | |
| SEMA3A | X | X | X | X | X | X | X | X | X | X |

TABLE B
Combination of at least three follicular cell markers

| | UGP2 & | UGP2 & | GAPBP1& |
|---|---|---|---|
| Gene | PHLDA1 | GAPBP1 | PHLDA1 |
| UGP2 | | | x |
| PHLDA1 | | x | |
| GAPBP1 | x | | x |

TABLE C
Combination of at least two cumulus cell markers

| gene | NRP1 | TOM1 | UBQLN1 | PSMD6 | DPP8 | HIST1H4C |
|---|---|---|---|---|---|---|
| TOM1 | x | | | | | |
| UBQLN1 | x | x | | | | |
| PSMD6 | x | x | x | | | |
| DPP8 | x | x | x | x | | |
| HIST1H4C | x | x | x | x | x | |
| CALM1 | x | x | x | x | x | x |

TABLE D
Combination of at least three cumulus cell markers

| | | TOM1 & | UBQLN1 & | PSMD6 & | | HIST1H4C & |
|---|---|---|---|---|---|---|
| gene | NRP1 & Tom1 | UBQLN1 | NRP1 | NRP1 | DPP8 &NRP1 | NRP1 |
| TOM1 | | | | x | x | x |
| UBQLN1 | x | | | x | x | x |
| PSMD6 | x | x | x | | x | x |
| DPP8 | x | x | x | x | | x |
| HIST1H4C | x | x | x | x | x | |
| CALM1 | x | x | x | x | x | x |

TABLE E
Combination of at least two or at least three follicular fluid markers

| | | | Ceruloplasmin & | ApoA-IV & | Ceruloplasmin & |
|---|---|---|---|---|---|
| protein | Ceruloplasmin | ApoA-IV | ApoA-IV | Beta-Actin | Beta actin |
| Ceruloplasmin | | | | x | |
| ApoA-IV | x | | | | x |
| Beta-Actin | x | x | x | | |

### Control Expression Level

In some embodiment, the methods of the invention further comprises comparing the expression level of the biological marker with a control expression level in control follicular fluid, cumulus cells or follicular cells sample. As used herein, “control expression level” is meant any value, including a predetermined value or a range of values, that is used for purposes of comparison. A control expression level can reflect the outcome of a single experiment or assay, or it can be a statistical function of multiple experiments or assays. A control expression level can also reflect the presence or absence of a signal. A control expression level can be generated from a prior measurement from the same subject or a measurement from a sample (e.g. follicular fluid, cumulus cells or follicular cells) from a single or from a pool of two or more oocytes competent for fertilization; from a single or from a pool of two or more oocytes not competent for fertilization; from a single or from a pool of two or more oocytes competent for embryo development; from a single or from a pool of two or more oocytes not.

Comparing the expression level of the biological marker with a control expression level may comprise comparing two values (or a set of values) in parallel, or comprise calculating a difference (e.g. a threshold level) or calculating a ratio in expression level(s). Such comparison may provide an absolute or relative gene/peptide expression. Whenever necessary, it is also possible to normalize the measured marker levels using available normalization tools, including using level of expression of the biological marker over level of expression of a housekeeping gene, including but not limited to ACTB, GAPDH and PPIA (Table 2C). It is within the knowledge of those skilled in the art to determine what measurements or controls are appropriate and which value(s) are acceptable to serve as control expression level(s).

According to some embodiments, when expression level of a marker in a tested follicular fluid, cumulus cells or follicular cells is lower than the average level of the same marker from the follicular fluid, cumulus cells or follicular cells originating from group of competent oocytes, it is deemed not likely competent to become fertilized or to implant. On the contrary, a tested follicular fluid, cumulus cells or follicular cells having an expression level of a marker similar or greater than the expression levels in the controls (competent group) will indicate that the oocyte is competent. Under such circumstances, the ratio of the expression level of a marker in a tested oocyte over the expression level of a marker in a control oocyte can be from about 1.5 above control to 150 (e.g. above 2, above 5, above 10, above 25, above 50, above 75, above 100 or more) and preferably above 2 for an oocyte to be deemed competent.

For some markers, it may be the opposite, i.e. a lower expression level of an ovarian marker in a tested follicular fluid, cumulus cells or follicular cells, when compared to appropriate controls (competent group) will indicate that the oocyte is competent. Under such circumstances, the ratio of the expression level of a marker in a control oocyte over the expression level of a marker of a tested oocyte may vary for instance from about 1.5 to 150 above control (e.g. above 2, above 5, above 10, above 25, above 50, above 75, above 100 or more).

Those skilled in the art will be able, when considering the instant disclosure to determine whether it is a higher or lower expression of the ovarian marker which is indicative of higher competency.

Those skilled in the art also understand that average expression level of one or more selected markers may be preferable to select or to assess oocytes competency, and more particularly oocytes likely to implant and to develop properly in the uterus up until the birth. For instance, in the case where the expression level of a marker in follicular fluid, cumulus cells or follicular cells of a tested oocyte is within the range associated with expression levels of competent oocytes (e.g. higher expression level compared to the range of incompetent oocytes) the tested oocyte will be deemed competent. On the contrary, if the level is below a defined or relative threshold then the oocyte will be considered incompetent or considered of lower potential.

### Competence Induction

Another aspect of the present invention relates to a method for improving oocyte competence. The method includes treating a subject with one or more factors known to modulate the expression one or more selected follicular fluid, cumulus cells or follicular cell markers according to the invention. The factor(s) is selected according to the markers and type of modulation that is desired (e.g. higher or lower levels of expression). For instance, administering a given hormonal treatment or a given schedule of treatment or a combination of dose and products (like FSH and LH) may increase the presence of markers and hence the competence of the resulting oocytes.

The markers according to the invention may also be useful to validate treatments aimed as contraceptive. For instance, if higher levels of a given marker is indicative of better chances of pregnancy, a lower level would indicate a lower chance of pregnancy. Therefore treatments aiming at reducing the presence of such a marker could be developed for contraceptive purposes. Methods of decreasing gene expression can be applied through various hormonal treatments or direct signaling path with specific chemicals such as phosphodiesterase inhibitors (e.g. Viagra™) or through RNAi or synthetic oligomer.

### Drug Screening

A further aspect of the present invention relates a method for screening candidate compounds capable of increasing or decreasing the expression of markers of the invention as described herein. For example, but not limited to, isolated cumulus or follicular cells put under in vitro culture conditions can be submitted to treatment with candidate compounds, and then tested for measuring the increase or decrease of expression levels of oocyte competence markers, therefore reflecting the effect of the candidate compound. This approach will allow the screening of compounds stimulatory or inhibitory to oocyte competence. The same compound testing can be performed under in vivo conditions, for instance following administration of a candidate compounds to subject, through which ovarian stimulation conditions can be tested for assessing expression of follicular fluid, cumulus cells or follicular cell markers according to the invention, and/or for assessing the production of competent oocytes.

According to a particular embodiment, the method for screening a compound stimulatory or inhibitory to mammalian oocyte competence comprises the steps of:
a) contacting follicular cells with a compound to be screened for activity to stimulate or inhibit the competence of an oocyte;
b) determining an expression level of at least one follicular cell marker in follicular cells contacted with said compound, wherein said at least one follicular cell marker is selected from the group consisting of UGP2, PHLDA1, GAPBP1, SFRP1, HOMER1, LRP8, DPYSL3, PGR, YWHAZ, MARCKS, SEMA3A, PIR, EREG and combinations thereof;
c) comparing the expression level measured in step b) with the expression level of non-contacted follicular cells;
   wherein a difference in the expression levels is indicative of the compound stimulatory or inhibitory effect.

According to another embodiment, the method for screening a compound stimulatory or inhibitory to mammalian oocyte competence comprises the steps of:
a) contacting cumulus cells with a compound to be screened for activity to stimulate or inhibit the competence of an oocyte;
b) determining an expression level of at least one cumulus cell marker contacted with the compound, wherein the at least one cumulus cell marker is selected from the group consisting of consisting of genes listed in Tables 4 to 8 and combinations thereof;
c) comparing the expression level measured in step b) with the expression level of non-contacted cumulus cells;
   wherein a difference in the expression levels is indicative of the compound stimulatory or inhibitory effect.

According to a further embodiment, the method for screening a compound stimulatory or inhibitory to mammalian oocyte competence comprises the steps of:
a) contacting follicular cells with a compound to be screened for activity to stimulate or inhibit the competence of an oocyte;
b) determining an expression level of at least one follicular cell marker in follicular cells contacted with the compound or in culture media deriving therefrom, wherein the at least one follicular cell marker is selected from the group consisting of Ceruloplasmin precursor, Apolipoprotein A-IV precursor, β-actin (ACTB) and combinations thereof;
c) comparing the expression level measured in step b) with the expression level of non-contacted follicular cells;
   wherein a difference in said expression levels is indicative of the compound stimulatory or inhibitory effect.

### Kits and Arrays

A further aspect of the invention relates to a solid support and to kits. The solid supports and/or kits of the invention may be useful for the practice of the methods of the invention, particularly for diagnostic applications in humans according to the evaluation methods described hereinbefore.

A solid support the invention may comprise a compound for assessing expression of one or more follicular fluid, cumulus cells or follicular cell markers as defined herein. In one embodiment, the compound is a nucleic acid probe designed for specific detection of a marker according to the invention. The solid support may me a tube, a chip (see for instance Affymetrix GeneChip® technology), a membrane, a glass support, a filter, a tissue culture dish, a polymeric material, a bead, a silica support, etc. The invention also encompasses the use of techniques and tools relating to microfluidic and lab-on-chip technology.

In some embodiment the solid support is a nucleic acid array. Nucleic acid arrays that are useful in the present invention include arrays such as those commercially available from Affymetrix (Santa Clara, Calif.), Applied Biosystems (Foster City, Calif.) and from Agilent Technologies (Santa Clara, Calif.). Preferred arrays according to the invention typically comprises a plurality of different nucleic acid probes (e.g. a probes capable of hybridization with a follicular fluid, cumulus cells or follicular cell markers as defined herein) that are coupled to a surface of a substrate in different, known locations. The array may be designed to detect sequences from an entire genome, or from one or more regions of a genome, for example selected regions of a genome such as those encoding for a protein or RNA of interest. Arrays according to the invention can be directed to a variety of purposes, including genotyping, diagnostics, mutation analysis, and marker expression. Arrays, also described as “microarrays” or “chips” may be produced and packaged using a variety of techniques known in the art.

According to a particular aspect, the invention relates to an array of nucleic acid probes immobilized on a solid support, the array comprising a plurality of probes hybridizing specifically to an ovarian marker associated with oocyte competency. The probes comprises a segment of at least twenty nucleotides exactly complementary to at least one reference sequence selected from the group of nucleic acid sequences encoding the genes listed in Tables 2A, 2B, 4 to 8 and 10.

A kit of the invention may comprise at least one oligonucleotide hybridizing specifically with an ovarian marker associated with oocyte competency (i.e. an ovarian marker comprising a sequence selected nucleic acid sequences encoding the genes listed in Tables 2A, 2B, 4 to 8 and 10). The kit may also comprise one or more additional components, such as a buffer for the homogenization of the biological sample(s), purified marker proteins (and/or a fragment thereof) to be used as controls, incubation buffer(s), substrate and assay buffer(s), standards, detection materials (e.g. antibodies, fluorescein-labelled derivatives, luminogenic substrates, detection solutions, scintillation counting fluid, etc.), laboratory supplies (e.g. desalting column, reaction tubes or microplates (e.g. 96- or 384-well plates), a user manual or instructions, etc. Preferably, the kit and methods of the invention are configured such as to permit a quantitative detection or measurement of the protein(s) or polynucleotide(s) of interest.

For instance, the kits may comprise at least one oligonucleotide which specifically hybridizes with nucleic acid molecules encoding any of the follicular fluid, cumulus cells or follicular cell markers defined herein, reaction buffers, and instructional material. Optionally, the at least one oligonucleotide contains a detectable tag. Certain kits may contain two such oligonucleotides, which serve as primers to amplify at least part of the markers. Some kits may contain a pair of oligonucleotides for detecting pre-characterized mutations in the follicular fluid, cumulus cells or follicular cell markers defined herein. Alternatively, the kit may comprise primers for amplifying at least part of the markers to allow for sequencing and identification of mutant nucleic acid molecules. The kits of the invention may also contain components of the amplification system, including PCR reaction materials such as buffers and a thermostable polymerase. In other embodiments, the kit of the present invention can be used in conjunction with commercially available amplification kits, such as may be obtained from GIBCO BRL (Gaithersburg, Md.) Stratagene (La Jolla, Calif.), Invitrogen (San Diego, Calif.). The kits may optionally include instructional material, positive or negative control reactions, templates, or markers, molecular weight size markers for gel electrophoresis, and the like.

Kits of the instant invention may also comprise antibodies immunologically specific for follicular fluid, cumulus cells or follicular cell markers defined herein and/or mutants thereof and instructional material. Optionally, the antibody contains a detectable tag. The kits may optionally include buffers for forming the immunocomplexes, agents for detecting the immunocomplexes, instructional material, solid supports, positive or negative control samples, molecular weight size markers for gel electrophoresis, and the like.

Those skilled in the art will recognize, or be able to ascertain using no more than routine experimentation, numerous equivalents to the specific procedures, embodiments, and examples described herein. Such equivalents are considered to be within the scope of this invention and covered by the claims appended hereto. The invention is further illustrated by the following examples, which should not be construed as further limiting.

### EXAMPLE 1

### Markers in Human Follicular Cells Associated with Competent Oocytes

### Materials and Methods

Follicular cells were obtained from women undergoing IVF treatments at the Fertility Center at the Ottawa Hospital, Canada, Women (n=18) with major indications for IVF, such as tubal infertility, unexplained infertility including endometriosis stage I/II/III and partners not requiring ICSI were recruited for the study. Patients with polycystic ovary syndrome (PCO), or partners with severe male factor requiring ICSI were not included in the study. The procedure was performed with the approval from the Ottawa Hospital Research Ethic Board.

Following ovarian stimulation, follicular fluid, follicular cells and oocytes from individual follicles were collected 36 h after hCG administration by ultrasound-guided follicular aspiration using a double lumen needle. The oocytes and surrounding cumulus cells were removed for IVF procedure. The Follicular cells recovery was performed as described previously (Hamel et al., 2008, Hum Reprod 23, 1118-27). After the recovering procedure, cells were rapidly frozen and stored in liquid nitrogen until RNA extraction.

### Treatment Assignment

Data (fertilization, embryo development, embryo morphology, transfer and pregnancy) generated from each follicle was recorded by an embryologist. Each embryo was scored according to the clinic's embryo selection protocol and based on main criteria, the cleavage stage and morphological characteristics (shape, size, granularity and 3D orientation of the blastomeres and space inside the zona pellucida occupied by enucleated fragments). Following a chart table, transferred embryos were at least 6-7 cells with high scores in morphology grade. Depending of the IVF protocol used, one or two embryos were transferred at either day 3 (16 patients) or day 5 (2 patients). Pregnancy was confirmed by the presence of a fetal heartbeat by ultrasound at 6 to 8 weeks.

For the hybridization experiments, were selected seven patients who produced follicular cells from oocytes that 100% resulted in a successful pregnancy (positive samples; n=9 follicles) and seven patients who produced follicular cells from oocytes that resulted in a transferred embryo with unsuccessful pregnancy (negative samples; n=9 follicles). For the Q-PCR, three pools of follicles [pool 1 (3 patients; n=3 follicles), pool 2 (4 patients; n=4 follicles) and pool 3 (4 patients, n=4 follicles)] were created from follicular cells associated with 100% of successful pregnancy which were called the pregnancy groups 1, 2 and 3 respectively. Three other pools [pool 1 (3 patients; n=3 follicles), pool 2 (4 patients; n=4 follicles) and pool 3 (4 patients, n=4 follicles)] were assigned to the no pregnancy groups 1, 2 and 3 respectively, containing follicular cells resulting in transferred embryos with unsuccessful pregnancy (Table 1).

TABLE 1
Treatment assignments with follicular cell tissues and patient characteristics for Q-PCR validation

| | Average | Average | Average | | | | |
|---|---|---|---|---|---|---|---|
| | number of | number of | number of | | | | |
| | oocytes | oocytes | embryos | | | Embryo | Pregnancy |
| | recovered | fertilized | transferred* | Pools | Patients | transfer | outcome |
| Pregnancy | 6.00 ± 1.344 | 3.33 ± 0.9428 | 1.22 ± 0.1470^{a} | Pool 1 | 1 | 1 | Single |
| groups | | | | | 2 | 1 | Single |
| | | | | | 3 | 1 | Twins |
| | | | | Pool 2 | 1 | 1 | Single |
| | | | | | 2 | 1 | Single |
| | | | | | 3 | 2 | Twins |
| | | | | Pool 3 | 1 | 1 | Single |
| | | | | | 2 | 1 | Single |
| | | | | | 3 | 2 | Twins |
| No | 6.89 ± 0.9782 | 4.00 ± 0.8498 | 1.89 ± 0.1111^{b} | Pool 1 | 1 | 1 | No Preg. |
| Pregnancy | | | | | 2 | 2 | No Preg. |
| groups | | | | | 3 | 2 | No Preg. |
| | | | | Pool 2 | 1 | 2 | No Preg. |
| | | | | | 2 | 2 | No Preg. |
| | | | | | 3 | 2 | No Preg. |
| | | | | Pool 3 | 1 | 2 | No Preg. |
| | | | | | 2 | 2 | No Preg. |
| | | | | | 3 | 2 | No Preg. |

*Values with different superscripts are different (p < 0.01)

### RNA Isolation

Total RNA from follicular cells was extracted with 1 ml of Trizol™ reagent (Invitrogen, Burlington, Canada) following the manufacturer's protocol. RNA was then further purified using the RNeasy™ total RNA clean-up protocol with the DNAse treatment (Qiagen, Mississauga, Canada). The concentration and integrity of the RNA samples were assessed spectrophotometrically at 260 nm and on an Agilent Bioanaliser 2100™ (Agilent Technology INC., Santa Clara, USA) running an aliquot for the RNA samples on the RNA 6000 Nano LabChip™. Only RNA that displayed intact 18S and 28S peaks was reverse transcribed to cDNA for hybridizations and Q-PCR experiments.

### Microarray Hybridizations

Total RNA of follicular cells was amplified using the RiboAmpT7™ RNA Amplification kit (Molecular Devices, USA)) according to the manufacturer's instructions. The RNA was submitted to one round of amplification and the quantity of aRNA was estimated by spectrophotometer at 260 nm. Probes were labelled with the ULS™ aRNA Fluorescent Labelling Kit (Kreatech Biotechnology, Salt Lake City, USA) according to the manufacturer's protocol, but without the aRNA fragmentation step. Slides were hybridized overnight at 50° C. with labelled purified probes using the SlideHyb™ #1 buffer (Ambion, Austin, USA). Hybridizations were performed in a SlideBooster™ using the Advacard AC3C™ (The Gel Company, San Francisco, USA). Slides were then washed twice with standard saline 2× citrate (SSC)/0.5% sodium dodecyl sulfate (SDS) for 15 min at 50° C. and twice with 0.5×SCC/0.5% SDS for 15 min at 50° C.

The hybridization was performed using positive group and negative group. The RNA from both positive and negative groups was used as probes with a dye swap manner. Slides were scanned using the VersArray ChipReader System™ (Bio-Rad) and analyzed using the ChipReader™ and ArrayPro Analyzer™ software (Media Cybernetics, Bethesda, USA). Fluorescence signal intensities for each replicate were log2 transformed, normalized by the Loess method, and corrected for background. The determination of the background signal threshold was performed with the SpotReport™ cDNA controls (Stratagene), which determine the background (t ¼m [symbol] 2_sd, where ‘t’ is the calculated threshold, ‘m’ the mean and ‘sd’ the standard deviation of the negative control data, n ¼ 58). Transcripts above the threshold were considered as present in follicular cells, whereas the other transcripts were eliminated from the analysis

### Candidate Gene Selection

Selection of clones for further analysis was based on the microarray results from the custom-made cDNA array slides and analysis performed with other hybridizations described previously (Hamel et al., 2008 supra). Markers were selected and graded according to their number of occurrences in different libraries, their repetition in the same library and the signal intensities.

Quantitative PCR

Primers of each candidate gene were designed with the Primer3TM web interface using sequences derived from The National Center for Biotechnology Information (NCBI) corresponding to our library sequences (Tables 2A, 2B and 2C). Real-time analysis measured and compared the three different groups of follicular cells for the pregnancy and no pregnancy groups with the same procedure already published (Vigneault et al., 2004, Biol Reprod. 2004 June; 70(6):1701-9). Briefly, for each sample, a reverse transcriptase was performed using 50 ng of granulosa cell RNA using the Sensiscript™ kit (Qiagen, Mississauga, Canada) according to the manufacturer's directions. To confirm that the right product was amplified, all amplifications were visualized on an agarose gel (2%) and then sequenced. Three housekeeping genes (β-actin (ACTB), cyclophylin A (PPIA) and glyceraldehyde-3-phosphate dehydrogenase (GAPDH) were used as an internal control.

TABLES 2A, 2B AND 2C
Sequences of specific primers used for amplification in Q-PCR

| | | | | | | | | |
|---|---|---|---|---|---|---|---|---|
| | Average | Average | | | | | | Fluorescence |
| | fold | fold | | GenBank | UniGene | Product | Annealing | acquisition |
| | change | change | | accession | accession | size | temperature | temperature |
| Genes | HYB A^{a} | HYB B^{b} | Primers sequences | number | number | (bp) | (° C.) | (° C.) |

Table 2A: Genes associated with competent follicle

| | | | | | | | | |
|---|---|---|---|---|---|---|---|---|
| EREG | 2.55 | 1.81 | Up 5′- | NM_001432 | Hs.115263 | 159 | 56 | 82 |
| | | | CTGGGTTTCCATCTTCTACAGG | | | | | |
| | | | (SEQ ID NO: 18) | | | | | |
| | | | Low 5′- | | | | | |
| | | | GCCATTCATGTCAGAGCTACAC | | | | | |
| | | | (SEQ ID NO: 19) | | | | | |
| DPYSL3 | 4.43 | 1.59 | Up 5′- | NM_001387 | Hs.519659 | 253 | 55 | 84 |
| | | | CAAAGTCAGTCTTGAGCAGAGG | | | | | |
| | | | (SEQ ID NO: 20) | | | | | |
| | | | Low 5′- | | | | | |
| | | | AGAAGCAGCATGATAGGGAAAG | | | | | |
| | | | (SEQ ID NO: 21) | | | | | |
| PGR | 2.79 | 1.69 | Up 5′- | NM_000926 | Hs.32405 | 149 | 54 | 82 |
| | | | GATTCAGAAGCCAGCCAGAG | | | | | |
| | | | (SEQ ID NO: 22) | | | | | |
| | | | Low 5′- | | | | | |
| | | | TACCTTCCATTGCCCTCTTAAA | | | | | |
| | | | (SEQ ID NO: 23) | | | | | |
| YWHAZ | 3.02 | 1.61 | Up 5′- | NM_145690 | Hs.492407 | 244 | 54 | 82 |
| | | | GGTGATGACAAGAAAGGGATTG | | | | | |
| | | | (SEQ ID NO: 24) | | | | | |
| | | | Low 5′- | | | | | |
| | | | GCGTGCTGTCTTTGTATGACTC | | | | | |
| | | | (SEQ ID NO: 25) | | | | | |
| MARCKS | 2.99 | 1.62 | Up 5′- | NM_002356 | Hs.519909 | 247 | 56 | 80 |
| | | | TTCTTCCTCTGCCTTGTTTCTC | | | | | |
| | | | (SEQ ID NO: 26) | | | | | |
| | | | Low 5′- | | | | | |
| | | | CAGCCTTTACACCATTTCTAGTG | | | | | |
| | | | (SEQ ID NO: 27) | | | | | |
| UGP2 | 2.98 | 1.61 | Up 5′- | NM_006759 | Hs.516217 | 163 | 55 | 77 |
| | | | GACTCAGTCGCACCAAGTTTC | | | | | |
| | | | (SEQ ID NO: 28) | | | | | |
| | | | Low 5′- | | | | | |
| | | | TTCCTCTCCTGCTTTAACAACC | | | | | |
| | | | (SEQ ID NO: 29) | | | | | |
| SEMA3A | 2.22 | 2.01 | Up 5′- | NM_006080 | Hs.252451 | 281 | 55 | 83 |
| | | | CAGCCCTGAAGAGAGAATCATC | | | | | |
| | | | (SEQ ID NO: 30) | | | | | |
| | | | Low 5′- | | | | | |
| | | | GCTCTGTGTCAATGACTTCCAG | | | | | |
| | | | (SEQ ID NO: 31) | | | | | |
| LRP8 | 2.24 | 2.60 | Up 5′- | NM_004631 | Hs.576154 | 258 | 54 | 82 |
| | | | CCAGAGTGCTCAGAAACTCAAG | | | | | |
| | | | (SEQ ID NO: 32) | | | | | |
| | | | Low 5′- | | | | | |
| | | | CCATCTTTATCTTCATCCACAGC | | | | | |
| | | | (SEQ ID NO: 33) | | | | | |
| PIR | 4.33 | 1.54 | Up 5′- | NM_003662 | Hs.495728 | 298 | 55 | 83 |
| | | | AACCCAGTAAGGATGGTGTGAC | | | | | |
| | | | (SEQ ID NO: 34) | | | | | |
| | | | Low 5′- | | | | | |
| | | | AAGACAAAGTGGCTTCTCTTGG | | | | | |
| | | | (SEQ ID NO: 35) | | | | | |
| PHLDA1 | 2.12 | 1.56 | Up 5′- | NM_007350 | Hs.602085 | 421 | 55 | 81 |
| | | | AGCTCAATAACTGTIGGGACAAAG | | | | | |
| | | | (SEQ ID NO: 36) | | | | | |
| | | | Low 5′- | | | | | |
| | | | CATGTGAGGGAAACTCCTTTAAGT | | | | | |
| | | | (SEQ ID NO: 37) | | | | | |

Table 2B: Genes associated with non competent follicle

| | | | | | | | | |
|---|---|---|---|---|---|---|---|---|
| SFRP1 | 3.05^{c} | 0 | Up 5′- | NM_003012 | Hs.713546 | 239 | 53 | 82 |
| | | | TTCTAATGATTGGCAAGTCACG | | | | | |
| | | | (SEQ ID NO: 38) | | | | | |
| | | | Low 5′- | | | | | |
| | | | TGGCTGATTCAGATTCAAAGAC | | | | | |
| | | | (SEQ ID NO: 39) | | | | | |
| HOMER1 | 3.05^{c} | 0 | Up 5′- | NM_004272 | Hs.591761 | 163 | 53 | 81 |
| | | | GGGAACAACCTATCTTCAGCAC | | | | | |
| | | | (SEQ ID NO: 40) | | | | | |
| | | | Low 5′- | | | | | |
| | | | ATTGCCTTTGAGCCATCTAAAC | | | | | |
| | | | (SEQ ID NO: 41) | | | | | |
| GABPB1 | 2.80^{c} | 0 | Up 5′- | NM_005254 | Hs.654350 | 223 | 53 | 81 |
| | | | TCCCACAGAAATGATGAAAGTG | | | | | |
| | | | (SEQ ID NO: 42) | | | | | |
| | | | Low 5′- | | | | | |
| | | | TGATGGAAGGCACAGAATACAG | | | | | |
| | | | (SEQ ID NO: 43) | | | | | |

Table 2C: Housekeeping genes

| | | | | | | |
|---|---|---|---|---|---|---|
| GAPDH | Up 5′- | NM_002046 | Hs.544577 | 452 | 56 | 89 |
| | ACCACAGTCCATGCCATCAC | | | | | |
| | (SEQ ID NO: 44) | | | | | |
| | Low 5′- | | | | | |
| | TCCACCACCCTGTTGCTGTA | | | | | |
| | (SEQ ID NO: 45) | | | | | |
| ACTB | Up 5′- | NM_001101 | Hs.520640 | 375 | 58 | 88 |
| | CGTGACATTAAGGAGAAGCTGTGC | | | | | |
| | (SEQ ID NO: 46) | | | | | |
| | Low 5′- | | | | | |
| | CTCAGGAGGAGCAATGATCTTGAT | | | | | |
| | (SEQ ID NO: 47) | | | | | |
| PPIA | Up 5′- | NM_007350 | Hs.602085 | 421 | 59 | 83 |
| | CAGCAGGCAGAGAAAATCCT | | | | | |
| | (SEQ ID NO: 48) | | | | | |
| | Low 5′- | | | | | |
| | GCATCTTTCGGGTTCCTTTT | | | | | |
| | (SEQ ID NO: 49) | | | | | |

^{abc}Fold change obtained by dye swap hybridizations.
EREG, Homo sapiens Epiregulin;
DPYSL3, Homo sapiens Dihydropyrimidinase-like 3;
PGR, Homo sapiens Progesterone receptor;
YWHAZ, Homo sapiens Tyrosine 3-monooxygenase/tryptophan 5-monooxygenase activation protein, zeta polypeptide;
MARCKS, Homo sapiens Myristoylated alanine-rich protein kinase C substrate;
UGP2, Homo sapiens UDP-glucose pyrophosphorylase 2;
SEMA3A, Homo sapiens Sema domain, immunoglobulin domain (Ig), short basic domain, secreted, (semaphorin) 3A;
LRP8, Homo sapiens Low density lipoprotein receptor-related protein 8, apolipoprotein e receptor;
PIR, Homo sapiens Pirin (iron-binding nuclear protein);
PHLDA1, Homo sapiens Pleckstrin homology-like domain, family A, member 1;
SFRP1, Homo sapiens Secreted frizzled-related protein 1;
HOMER1, Homo sapiens Homer homolog 1 (Drosophila);
GABPB1, Homo sapiens GA binding protein transcription factor, beta subunit 1;
GAPDH, Homo sapiensGlyceraldehyde-3-phophate dehydrogenase;
ACTB, Homo sapiens Beta actin;
PPIA, Homo sapiens cyclophylin A.

### Statistical Analysis

Analysis of the gene expression stability over the different positive and negative samples was performed using the GeNorm VBA™ applet software. This analysis relies on the principle that the expression ratio of two ideal internal control genes is identical in all samples, regardless of the experimental condition or cell type, and determined as the standard deviation of the logarithmically transformed expression ratios. Using the software, the internal control gene stability (the M value) was calculated as the average pair wise variation of a particular gene (ACTB, PPIA and GAPDH in this study) with respect to the rest of the genes, and ranking was made based on these values. The most stable reference genes were identified by stepwise exclusions of the least stable gene and recalculating the M values. Following GeNorm™ analysis, the actin and GAPDH were the most stable genes and the M values was less than 1.5 as the software recommendation (M values=0.634). Normalization of genes was calculated according the normalization factors for each sample. Data are presented as mean+SEM. The evaluation of mRNA differences between the positive groups and negative groups was done by a non-parametric two-tailed unpaired t-test. Differences were considered statistically significant at the 95% confidence level (P<0.05) and a tendency at the 90% level (P<0.1).

### Results

Data generated from each follicle (fertilization, embryo development, embryo morphology, transfer, and pregnancy) were recorded by an embryologist. From patients recruited for the study (Table 1), we selected patients whom had 100% transferred embryos associated with successful pregnancies (pregnancy groups) and patients who had 100% transferred embryos associated with unsuccessful pregnancy (no pregnancy group). In pregnancy groups, all double embryo transfers have resulted in a double pregnancy (twin) and a single embryo transfer have resulted in a twin pregnancy for one patient of the pool 1. Average numbers of oocytes recovered and fertilized were similar in both groups, but the average number of embryo transferred was higher in no pregnancy group (P=0.0023).

### Microarray Hybridizations

Hybridizations with RNA from follicular cells were performed. A total of 62 transcripts of the total transcripts have demonstrated ratio (>2.0) preferentially expressed in the Pregnancy Group. Hybridizations comparison from follicular cells from follicles leading to a pregnancy already resulted in the identification of 31 common transcripts coding for 25 different genes. For the transcripts preferentially expressed in the No Pregnancy group, we detected 54 transcripts with ratios>2.0.

### Candidate Genes Selection

From the 25 candidate genes expressed preferentially in the Pregnancy group (hybridization A) and in the two other hybridizations (hybridizations B), markers were selected and graded according to their known function, their number of occurrences in different libraries, their repetition in the same library and the signal intensity (fold changes>2.00 for hybridization A and >1.5 for hybridizations B). After selection and gradation, 10 candidate genes were validated by Q-PCR (Table 2A): Epiregulin (EREG), Dihydropyrimidinase-like 3 (DPYSL3), Progesterone receptor (PGR), Tyrosine 3-monooxygenase/tryptophan 5-monooxygenase activation protein (YWHAZ), Myristoylated alanine-rich protein kinase C substrate (MARCKS), UDP-glucose pyrophosphorylase 2 (UGP2), semaphorin 3A (SEMA3A), low density lipoprotein receptor-related protein (LRP8), Pirin (PIR) and Pleckstrin homology-like domain, family A, member 1 (PHLDA1).

From the 54 transcripts preferentially expressed in the No Pregnancy group in hybridization A (fold change>2.00), 24 transcripts were not expressed in hybridizations B previously done from follicular cells from follicles leading to a pregnancy. A total of 19 different genes were found. Candidate markers were selected and graded according to their known function and the signal intensity. After selection and gradation, 3 candidate genes from Non Pregnancy Group were validated by Q-PCR (Table 2B): Secreted frizzled-related protein 1 (SFRP1), homer homolog 1 (HOMER1) and GA binding protein transcription factor β1(GABPB1).

### Quantitative PCR

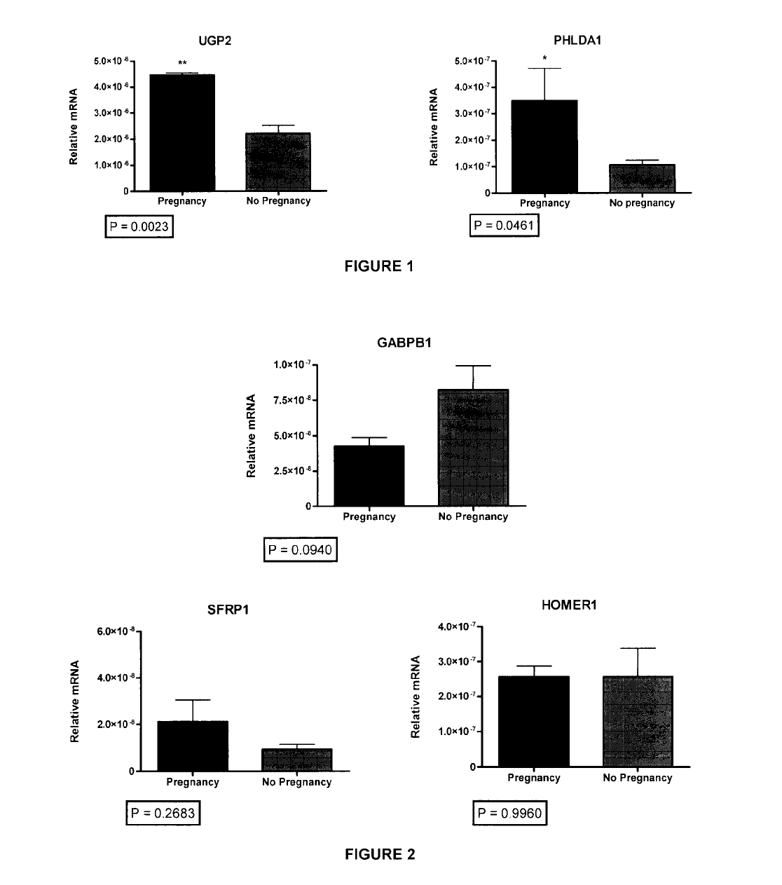

Quantitative PCR was performed with all three pools of human follicular cells from each group (pregnancy and non pregnancy groups) (Table 1). From the 10 candidate genes selected as indicator of pregnancy, two genes [UGP2 (P=0.0023) and PHLDA1 (P=0.0461)] had a statistical difference between follicular cells of pregnancy and non pregnancy groups (P<0.05) (FIG. 1). The UGP2 (P=0.0023) had a higher gene expression in the pregnancy groups (P<0.01) (FIG. 1). No differences in the mRNA levels were observed between the two groups in the eight other selected genes (Table 3). The expression of housekeeping genes ACTB, GAPDH and PPIA was similar (P>0.05) in both groups (Table 3).

TABLE 3
Quantification of mRNA level by Q-PCR that showed similar expression (P > 0.05)
in follicular cells from follicles associated with or without a pregnancy.

| | | | | No | | |
|---|---|---|---|---|---|---|
| | Mean | Pregnancy | Mean No | Pregnancy | | |
| | pregnancy | groups +/− | Pregnancy | groups +/− | Difference | P |
| Gene | groups (fg) | SEM | groups (fg) | SEM | between means | Value |
| SEMA3A | 6.60E−07 | 1.88E−07 | 2.44E−07 | 1.49E−07 | 4.17E−07 ± 2.40E−07 | 0.1564 |
| YWHAZ | 2.13E−07 | 2.26E−08 | 1.41E−07 | 4.54E−08 | 7.29E−08 ± 5.07E−08 | 0.2242 |
| PGR | 6.68E−09 | 1.72E−09 | 5.01E−09 | 8.94E−10 | 1.66E−09 ± 1.94E−09 | 0.4397 |
| LRP8 | 3.91E−09 | 2.10E−09 | 3.79E−09 | 2.01E−09 | 1.22E−10 ± 2.91E−09 | 0.4782 |
| MARCKS | 1.56E−07 | 1.35E−07 | 2.56E−07 | 1.51E−07 | −1.00E−07 ± 2.03E−07 | 0.6473 |
| PIR | 9.15E−08 | 1.79E−08 | 1.12E−07 | 4.41E−08 | −2.00E−08 ± 4.76E−08 | 0.6956 |
| DPYSL3 | 7.83E−08 | 1.85E−08 | 8.84E−08 | 2.94E−08 | −1.01E−08 ± 3.47E−08 | 0.7850 |
| EREG | 5.80E−09 | 3.34E−09 | 5.59E−09 | 2.38E−09 | 2.07E−10 ± 4.10E−09 | 0.9622 |
| GAPDH | 8.80E−07 | 1.16E−07 | 6.48E−07 | 9.86E−08 | 2.33E−07 ± 1.52E−07 | 0.2008 |
| CYC | 6.45E−06 | 2.79E−06 | 8.14E−06 | 3.22E−06 | 1.69E−06 ± 4.26E−06 | 0.7116 |
| ACTB | 3.64E−06 | 5.17E−07 | 3.21E−06 | 1.03E−06 | 4.27E−07 ± 1.16E−06 | 0.7308 |

From the 3 candidate genes selected as indicator of no pregnancy, one gene [GABPB1 (P=0.0940)] (FIG. 2) was not statistically different between the two groups, mainly due to larger variation in the levels measured, but could be considered as potential indicators of follicular incompetence. No differences in the transcripts levels were observed for the two other genes selected (FIG. 2).

### EXAMPLE 2

### Markers in Human Cumulus Cells Associated with Competent Oocytes

### Materials and Methods

### Patient Selection

Eight consent patients (n=8) were selected for this study at the IVF clinic of the medical faculty of the University of Bonn based on the diagnosis of male factor infertility with reduced sperm quality.

Ovarian Stimulation and Cumulus-oocyte Complex (COG) etrieval

Ovarian stimulation started with the administration of the gonadotrophin releasing hormone agonist (GnRHa) triptorelin acetate (Decapeptyl (0.1 mg/day), Ferring, Germany) since the 22nd day of the preceding oestral cycle. Daily administration of the human menopausal gonadotrophin (HMG; Humegon, Organon) and/or follicular stimulating hormone (FSH; Fertinorm, Serono) was carried out 12 to 15 day later. The HMG/FSH (225 IU) dose was adjusted through a transvaginal ultrasound monitoring of the patient's individual response mainly the follicular size and oestradiol levels. When some follicles of the ovulatory wave go beyond 18 mm in diameter, human chorionic gonadotrophin (HCG; 10000 IU) was administered and 36 to 38 h later cumulus-oocyte complexes (COCs) were transvaginally punctured. All the protocols used herein were approved by the institutional review board of the medical faculty of the University of Bonn.

### Cumulus Cells Collection and Zona Birefringence Analysis:

Following follicular aspiration, collected COCs were immediately washed in HEPES-buffered medium (Cook, Brisbane, Australia) and individually cultured in IVF-20™ media (Scandinavian IVF Sciences AB, SIVFS, Göteborg, Sweden) for two hours. Incubation was performed in a mini-incubator (Minc, Cook) using pre-mixed gas with low oxygen (6% CO₂, 5% O₂, 89% N₂) at 37° C.

Each COC was put in a dish containing a Hepes-buffered medium under oil. Cumulus cells (CCs) were dissected using sterile scalpel and transferred immediately into a sterile tube and stored at −80° C. for further analysis. After that, a hyaluronidase treatment to remove the remaining cumuli was achieved and denuded oocytes were individually incubated at 37° C. in 5-μl droplets of IVF-20™ medium covered with mineral oil in a glass-bottom dish (Willco, Wells BV dish, MTG, Altdorf, Germany) for 1 to 2 hour. Oocytes with vacuolization were excluded and neither used for zona imaging nor for ICSI. Prior switching to the birefringence analysis mode to assess the zona score, immature oocytes (absence of the first polar body when scanned by the conventional light microscopy) were also removed. As described previously by Montag et al. (Montag et al. (2007), Reprod Biomed Online 16, 239-44). Unfertilized MII oocytes were classified based on their inner zona layer birefringence measurement using an automatic module Octax polairAide™ (Octax ICSI Guard™, OCTAX Microscience GmbH, Altdorf, Germany) connected to a polarization imaging software (OCTAX Eyeware™) that recorded images combining bright field (green) and birefringence (red). Zona score was therefore automatically and non-invasively measured in a real time way based on the intensity and the uniformity of the birefringence at 180 measuring points of the inner zona layer. The temperature of the heated plate was linked to a calibrated sensor to maintain 37.0±0.5° C. in the medium droplet during microscopic observation. A micromanipulation system (Eppendorf, Hamburg, Germany) adapted to the microscope allowed rotation of oocytes to optimize zona visualization and scoring. MII oocytes with an irregular and/or low birefringence distribution in the inner zona layer were classified as low zona birefringence (LZB). However, those with a high-intensity and uniform birefringent inner zona layer were classified as high zona birefringence (HZB). The highest priority in ICSI and later embryo transfer was given to the MII oocytes with uniformly bright and very thick inner zona layer.

### Intracytoplasmic Sperm Injection

All media used for oocyte retrieval, denuding, ICSI treatment and subsequent culture were of pharmaceutical grade, free of phenol red and provided by SIVFS company (IVF-50™; Gamete-100™, ICSI-1; Scandinavian IVF Science, Göteborg, Sweden). The selection of patients for ICSI treatment was based on the diagnosis of male factor infertility due to reduced sperm quality. In a collaborative approach, all patients underwent an extensive andrological, gynecological and cytogenetic examination prior to ICSI to avoid any other bias.

ICSI was performed within 1 h after zona imaging. Oocytes were kept in the same order as during zona imaging and thereafter cultured individually in 30-μl medium droplets under oil. The spermatozoa ejaculate was first diluted by a mini-swim-up technique, then washed first with Gamete-100 buffer and finally with 1 ml of IVF50™ medium. After each wash step, a centrifugation step in a microfuge (Biofuge 13™, Heraeus, Osterode, Germany) was achieved. The sperm final pellet was resuspended in 20-50 ml of IVF50™ and stored in a CO₂ incubator. A few microlitres of the motile sperm suspension were placed into a central polyvinylpyrrolidone (PVP) droplet (ICSI-1) in the injection dish. ICSI was carried out on the heated stage of an inverted microscope (DMIRB; Leica, Bensheim, Germany) equipped with microinjection devices for holding the oocyte and sperm injection (Narishige, Tokyo, Japan). All MII oocytes were fertilized by ICSI. Following injection, oocytes were cultured in IVF-50™ up to the time of transfer.

### Embryo Culture and Transfer

Eighteen (18) hours following ICSI, oocytes with two pronuclei (the two polar nuclei; 2PN) of equal size in close proximity and centrally located within the ooplasm were considered as successfully fertilized. Among them and due to legal restrictions, only two fertilized oocytes were chosen for transfer. The principal criterion for selection was the intensity of zona birefringence (the two top zone scorer were taken). Ideally, two oocytes with initially HZB were chosen for further embryo culture and transfer; whereas the supernumerary oocytes were cryopreserved. The selected 2×2PN were further individually cultured until transfer on day 3 using the Cook™ culture system (COOK, Brisbane, Australia). Incubation was done in a Minc™ benchtop incubator at 5% O₂, 6% CO₂, 98% N₂. Transvaginal intrauterine embryo transfer was done with 30 μl culture medium using a Sydney IVF™ catheter (COOK, Brisbane, Australia) as described previously (Montag et al., 2002, Eur J Obstet Gynecol Reprod Biol 102, 57-60). Progesterone vaginal suppositories (200 mg/day) were used twice a day to support the luteal phase. This treatment began on the subsequent day following the HCG administration. Pregnancy was assessed first through a positive HCG test at day 14 after transfer and then a higher value 2 days later. Proven implantation and pregnancy were thereafter confirmed by ultrasonic detection of gestational sacs and a positive heart beat (viable embryo) 3 weeks later.

### Patient's Groups

Based on the pregnancy results, individual cumulus cells from the eight (n=8) patients were divided into two main groups to explore in vivo genomic markers expressed in CC and associated to oocyte competence, embryo quality as well as pregnancy. The CC of the zona good oocytes with successful pregnancy (ZGP) was the first group. It includes 8 cumuli of individual oocytes (from 4 patients) that lead to pregnancy. However, the second group contains 6 individual cumuli of individual oocytes (again from different 4 patients) with zona good score but an unsuccessful pregnancy (ZGNP). While ZGP represents the positive group, the ZGNP is the negative one.

Custom-made cDNA Microarray Preparation

Four suppressive subtractive cDNA hybridizations (SSH) previously achieved in our lab were printed on our custom-made microarray. Differentially expressed cDNA associated to in vivo competent oocytes of human follicular cells (Hamel et al., 2008 supra), human cumulus cells (Hamel et al., 2008), bovine granulosa cells (Robert et al., 2001, Biol Reprod 64, 1812-20) and bovine cumulus cells (Assidi et al., 2008, Biol Reprod 79, 209-22) were amplified, purified, sequenced and identified through their blast against the cDNA Library Manager Program (Genome Canada bioinformatics, Quebec, Canada). SSH products, negative and positive controls were dissolved in equal volumes of dimethyl sulfoxide (DMSO) and H₂O, and spotted in two replicates in different locations on GAPSII glass slides (Corning, Corning, N.Y., United States) using VersArray Chip WriterPro™ robot (Bio-Rad, Mississauga, Canada) as detailed elsewhere (Assidi et al., 2008, Biol Reprod 79, 209-22). UV rays served to cross-link the oligonucleotides before the Terminal Deoxynucleotidyl Transferase quality control Assay (GE healthcare, Quebec, Canada).

### Total RNA Extraction:

The cumulus cells samples of each oocyte in both experimental groups were subjected to total RNA extraction using the PicoPure™ RNA Isolation Kit (Arcturus, Molecular Devices Analytical Technologies, Sunnyvale, Calif., USA) according to the manufacturer's instructions. Briefly, cumulus cells were extracted in 100 μl of extraction Buffer (XB), incubated for 30 min at 42° C. and centrifuged 2 min at 3000 g. The supernatant containing the RNA was collected, mixed with an equal volume of 70% ethanol, transferred to a previously conditioned purification column and spun for 1 min. To prevent contamination and immediately after a first wash with 100 μl of w1 wash buffer, an on-Column DNase Digestion for 15 min on benchtop with the RNase-Free DNase Set (Qiagen, Maryland, USA) and according to the manufacturers' instructions. Following the two washing steps respectively with buffer w1 and w2 provided, the column product was resuspended in 30 μl of elution buffer (EB) provided in the kit. The concentration and quality of the RNA were assessed by the Agilent 2100™ bioanalyzer (Agilent Technologies, Waldbronn, Germany) according to the manufacturer's protocol.

### Messenger RNA Linear Amplification:

Based on RNA concentrations of each individual CC (biological replicate), 10 ng of total RNA were pooled for each experimental groups (pool of 8 and 6 replicates respectively for the pregnant and the non-pregnant group) for amplification using 2-round in vitro transcription (IVT) following the instructions of the RiboAmp^{plus}™ RNA Amplification kit (Arcturus, Molecular Devices Analytical Technologies, Sunnyvale, Calif., USA). Briefly, RNA was first reversed transcribed with the incorporation of a primer containing a T7 RNA polymerase promoter sequence (RiboAmp™ primer A). Double-stranded cDNA was then synthesized, column-purified and used as a template that drives the first 6-hour round of the T7-polymerase IVT. One microliter of this elution was used for the NanoDrop™ (NanoDrop Technologies, Wilmington, Del., USA) quantification of the first round yield, whereas the rest served as a template for the second round. Similarly to round 1, the second linear amplification round was carried out according to the kit recommendations and the resulting RNA was column-purified and eluted in 30 μl of RNA eluted buffer (RE). The final RNA amplification yield was quantified by spectrophotometry at 260 nm using the NanoDrop ND1000™ (NanoDrop Technologies) as before.

### Messenger RNA Indirect Labelling:

Amplified Messenger RNA of each group (ZGP vs ZGNP) was divided into 2 sub-replicates per chip type (FIG. 1) to get a dye-swap design and labelled using the Universal Linkage System (ULS™) aRNA Fluorescent Labelling Kit (KREATECH Biotechnology, Amsterdam, The Netherlands) according to the manufacturer's instructions. Briefly, for each sub-replicate, 2.5 μg of amplified RNA was labelled by incubation with 2.5 μl of Cy5/DY647-ULS or Cy3/DY547-ULS dyes, and 2 μl of 10× labelling solution in a 20-μl total volume at 85° C. for 15 min. Unbound dye was then removed, as recommended, using the KREApure™ columns provided in the kit. Labelled RNA was quantified on the NanoDrop ND-1000™ (NanoDrop Technologies). Each probe of the ZGP group was mixed with its correspondent one in the ZGNP group in in equimolar proportions before hybridization.

### Hybridization Design:

Custom-made cDNA Microarray Hybridizations

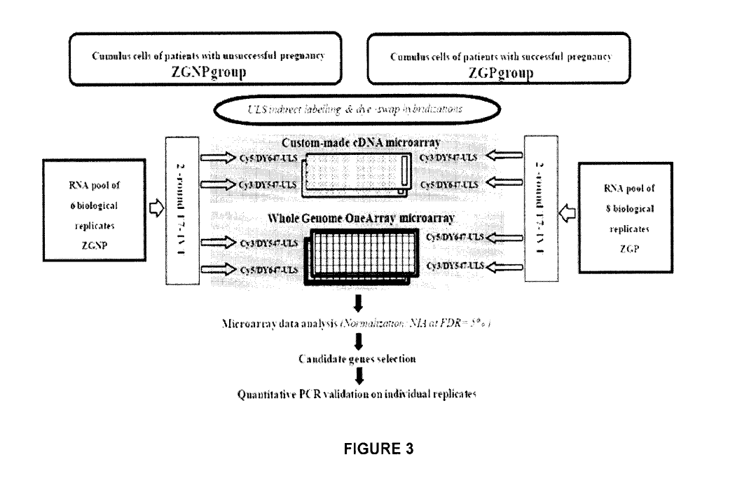

Two hybridizations were performed in a dye-swap design (FIG. 1) on our custom-made array. Hybridizations were performed in the ArrayBooster™ using the Advacard AC3C™ (The Gel Company, San Francisco, Calif., USA) for 18 h at 50° C. using Slide Hyb#1™ (Ambion, Austin, Tex.). The slides were then washed twice in 2×SSC/0.5% SDS buffer, and twice in 0.5×SSC/0.5% SDS buffer. After two quick final washes at room temperature in 1×SSC and water, slides were spin-dried, scanned and analyzed using the ChipReader™ and ArrayPro Analyzer™ software (Media Cybernetics, San Diego, Calif., USA). FIG. 3 provides a visual description of the design.

Hybridizations using the OneArray™ 30K 60-mer Oligo Microarray

In order to achieve a large scale candidates search, two additional hybridizations in a dye-swap design (FIG. 1) were made using two OneArray™ microarrays (Phalanx Biotech, Palo Alto, Calif., USA). It is a 60-mer sense-strand polynucleotide microarray that contains 30,968 of highly sensitive and specific human probes and 1082 experimental control probes. After a prehybridization step of 10 min at 60° C., the hybridization protocol was performed according the DNA microarray user guide (Phalanx Biotech; available on the website of the company) and using the recommended hybridization and washing buffers.

### Hybridization Data Analysis

Following hybridization, both microarray slides were scanned using the VersArray ChipReader™3.1 System (Bio-Rad, Mississauga, Canada) and analyzed using the ArrayPro Analyzer™ software (Media Cybernetics, Bethesda, USA). Raw microarray data were first Loess-normalized and corrected for background as described elsewhere (Assidi et al., 2008, Biol Reprod 79, 209-22). Ratio of net fluorescence intensities of our dye-swap experiments between positive (pregnant) and negative (non pregnant) group was analyzed using the free-software National Institute on Aging (NIA) Array Analysis Tool (Baltimore, Md., USA) developed at NIA (NIA Array Analysis Tool, 2009, National Institute on Aging (NIA/NIH), Laboratory of Genetics, Baltimore Md., USA. http://lgsun.grania.nih.gov/ANOVA/.) at FDR=5% and a minimum cut off limit of 2.25. Since each clone was printed twice on our slide (Hamel et al., 2008, Hum Reprod 23, 1118-27), two additional technical sub-replicates that emerged from this design were taken into account during the statistical analysis. Two lists of more than two-fold change in both over-expressed and under-expressed clones in the ZGP group compared to the ZGNP one were generated for subsequent analysis to define suitable markers expressed in cumulus cells and associated with good quality oocytes.

### Real-Time PCR Validation

Equal amount of total RNA were taken from each replicate on individual CC of each patient group. To denature the RNA and remove secondary structures, the RNAs were heated at 65° C. for 5 min and then quenched rapidly on ice for at least two minutes. Samples were then reversed transcribed using the Sensi Script™ reverse transcriptase kit (Qiagen, Mississauga, ON, Canada) according to the manufacturer's recommendations. Real time PCR was performed on the 17 selected candidates from both hybridizations of our custom-made cDNA array and the 60-mer oligonucleotide OneArray™ chip in LightCycler™ capillaries (Roche Applied Science, Mannheim, Germany) using the LightCycler™ FastStart™ DNA Master™ SYBR Green I (Roche) as detailed elsewhere (Assidi et al., 2008, Biol Reprod 79, 209-22). For each candidate, specific set of primers were designed using the NCBI's primer-blast software and the candidates specific sequences (NCBI) (table 1). Additionally, three housekeeping genes ACTB (β-Actin), GAPDH, and PPIA were quantified and used in Genorm Normalization™. The two most stable housekeepings (ACTB and PPIA; P>0.05) in both groups were maintained as the suitable control genes for QPCR data normalization. The real-time PCR product specificity of each candidate was confirmed by the sequencing to validate the amplification of the appropriate product as well as by the analysis of the LightCycler™ melting curve (Roche). Each gene mRNA expression level was then divided by its normalization factor and log-transformed. A t-test to compare gene expression levels between both groups was thereafter performed using the GraphPad Prism 5™ software (GraphPad Software, San Diego, Calif., USA) at α=0.05.

Table 4 hereinafter shows the sequences of specific primers of candidates used in real time PCR quantification.

TABLE 4
Sequences of specific primers of candidates used in real time PCR quantification

| | | | | | | Fluorescence |
|---|---|---|---|---|---|---|
| Gene | | | | | Annealing | acquisition |
| Name* | Primer set (5′-3′) | | Genbank | Unigene | temperature | temperature |
| CALU | Up | 5′- ACAAGGATGGAGACCTCATTGCC -3′ | AF013759 | Hs.7753 | 62 | 80 |
| | | (SEQ ID NO: 50) | | | | |
| | low | 5′- TGCTCTCGCTCTGTCTTTACCC -3′ | | | | |
| | | (SEQ ID NO: 51) | | | | |
| DPP8 | Up | 5′- GCTGCCTGCTCCAAGTGATTTCAA -3′ | NM_197960.2 | Hs.591106 | 61 | 83 |
| | | (SEQ ID NO: 52) | | | | |
| | low | 5′- GCAAGAATGTGAGTAGCCACGGT -3′ | | | | |
| | | (SEQ ID NO: 53) | | | | |
| HIST1H4C | Up | 5′- CCATCGTAAGGTGCTCCG -3′ | NM_003542 | Hs.46423 | 60 | 81 |
| | | (SEQ ID NO: 54) | | | | |
| | low | 5′- TTGGCGTGCTCCGTATAGGT -3′ | | | | |
| | | (SEQ ID NO: 55) | | | | |
| PKN2 | Up | 5′- ACAAGCCTGATACTCCTCAGTCAG -3′ | NM_006256.2 | Hs.440833 | 61 | 81 |
| | | (SEQ ID NO: 56) | | | | |
| | low | 5′- GCAACCCAAGAACTACACAAGCAG -3′ | | | | |
| | | (SEQ ID NO: 57) | | | | |
| PSMD6 | Up | 5′- TTCCAGCAGTTCGGCAGTATCTGT -3′ | BC000630 | Hs.152536 | 61 | 81 |
| | | (SEQ ID NO: 58) | | | | |
| | low | 5′- TTCCACACCAACACCAAACGCTTC -3′ | | | | |
| | | (SEQ ID NO: 59) | | | | |
| RPL9 | Up | 5′- TGAAGGGACGCACAGTTATCGTGA -3′ | BC066318 | Hs.719072 | 60 | 83 |
| | | (SEQ ID NO: 60) | | | | |
| | low | 5′- AAGCAACACCTGGTCTCATCCGAA -3′ | | | | |
| | | (SEQ ID NO: 61) | | | | |
| SYT11 | Up | 5′- ACCCTGTGTTTGACGAGACCTTCA -3′ | BC039205 | Hs.32984 | 63 | 87 |
| | | (SEQ ID NO: 62) | | | | |
| | low | 5′- CATCTTCGGCAAGTGTCTGGCTTT -3′ | | | | |
| | | (SEQ ID NO: 63) | | | | |
| THOC2 | Up | 5′- GGTAATCTTTCAGGAAGGTGGAGA -3′ | NM_001081550.1 | Hs.592243 | 60 | 81 |
| | | (SEQ ID NO: 64) | | | | |
| | low | 5′- GCTGATGTCATCCCAGACTTTG -3′ | | | | |
| | | (SEQ ID NO: 65 | | | | |
| TOM1 | Up | 5′- CAATCTCAACAATGTGTTCCTGCG -3′ | NM_001135732 | Hs.474705 | 60 | 77 |
| | | (SEQ ID NO: 66) | | | | |
| | low | 5′- TACCTCTTTCCGTTGGTCAGCC -3′ | | | | |
| | | (SEQ ID NO: 67) | | | | |
| TUG1 | Up | 5′- CTTCAGATCAGCAGGACAGTTGG -3′ | NR_002323.1 | Hs.554829 | 62 | 80 |
| | | (SEQ ID NO: 68) | | | | |
| | low | 5′- GGGAGTTGTTACAAGATGGAACGG -3′ | | | | |
| | | (SEQ ID NO: 69) | | | | |
| AR | Up | 5′- AACCCTATTTCCCCACCCCAG -3′ | NM_000044.2 | Hs.496240 | 59 | 83 |
| | | (SEQ ID NO: 70 | | | | |
| | low | 5′- GCTCTCTAAACTTCCCGTGGCA -3′ | | | | |
| | | (SEQ ID NO: 71) | | | | |
| CALM1 | Up | 5′- TACTTCGTGTGCTCCGACCCAT -3′ | BC007965 | Hs.282410 | 60 | 84 |
| | | (SEQ ID NO: 72) | | | | |
| | low | 5′- AGTCCACAGCCACAGCCTACTC -3′ | | | | |
| | | (SEQ ID NO: 73) | | | | |
| NRP1 | Up | 5′- ACCTGAAACCCAGTGCCCAGAA -3′ | NM_003873.5 | Hs.131704 | 60 | 84 |
| | | (SEQ ID NO: 74) | | | | |
| | low | 5′- TGTTGTTGCGGTTGTCAGCAGT -3′ | | | | |
| | | (SEQ ID NO: 75) | | | | |
| CHD9 | Up | 5′- ACCAGCCTCGTCAATTTCCCAA -3′ | NM_025134.4 | Hs.622347 | 59 | 80 |
| | | (SEQ ID NO: 76) | | | | |
| | low | 5′- CATCTCCTGCAAGTCTCGTTCCA -3′ | | | | |
| | | (SEQ ID NO: 77) | | | | |
| SPHKAP | Up | 5′- GCAGCGATTTGCCTTGACAAC -3′ | NM_001142644.1 | Hs.436306 | 58 | 85 |
| | | (SEQ ID NO: 78) | | | | |
| | low | 5′- TTTAAGCTCAGGGTGCTCGTCC -3′ | | | | |
| | | (SEQ ID NO: 79) | | | | |
| CHGB | Up | 5′- CAACTGGACCAGCTCCTTCAC -3′ | NM_001819.2 | Hs.516874 | 58 | 85 |
| | | (SEQ ID NO: 80) | | | | |
| | low | 5′- GCACAGTCATTGTCATAAGCATGT -3′ | | | | |
| | | (SEQ ID NO: 81) | | | | |
| UBQLN1 | Up | 5′- CAGTGATACATTTGGCTGACTCTGG -3′ | BC017289 | Hs.9589 | 62 | 82 |
| | | (SEQ ID NO: 82) | | | | |
| | low | 5′- GCCTCCACCGTAACCTTTGTACTT -3′ | | | | |
| | | (SEQ ID NO: 83) | | | | |
| PPIA | Up | 5′- TGCTGGACCCAACACAAATGGTTC -3′ | NM_021130.3 | Hs.356331 | 60 | 84 |
| | | (SEQ ID NO: 84) | | | | |
| | low | 5′- TGGTGATCTTCTTGCTGGTCTTGC -3′ | | | | |
| | | (SEQ ID NO: 85) | | | | |
| ACTB | Up | 5′- CGTGACATTAAGGAGAAGCTGTGC -3′ | NM_001101 | Hs.520640 | 59 | 89 |
| | | (SEQ ID NO: 86) | | | | |
| | low | 5′- CTCAGGAGGAGCAATGATCTTGAT -3′ | | | | |
| | | (SEQ ID NO: 87) | | | | |

*CALU = calumenin;
CALM1 = calmodulin 1 (phosphorylase kinase, delta);
TUG1 = taurine upregulated 1;
CHD9 = chromodomain helicase DNA binding protein 9;
PSMD6 = proteasome (prosome, macropain) 26S subunit, non-ATPase, 6;
UBQLN1 = ubiquilin 1;
AR = androgen receptor;
SYT11 = synaptotagmin XI;
PKN2 = protein kinase N2;
NRP1 = neuropilin 1;
RPL9 = ribosomal protein L9;
THOC2 = THO complex 2;
DPP8 = dipeptidyl-peptidase 8;
HIST1H4C = histone cluster 1, H4c;
TOM1 = target of myb1 (chicken);
SPHKAP = SPHK1(sphingosine kinase type 1) interactor, AKAP domain containing;
CHGB = chromogranin B (secretogranin 1)

### Results

### Microarray Data Analysis:

Following microarray experiment analysis, candidate gene selection was achieved based on the microarray results from both our custom-made cDNA array and the OneArray™ slides. By comparing the positive clone lists from the two different groups, two main categories of candidates were selected based on their fold change (fold>2, FDR=5%). The first category corresponds to the competence markers and includes 260 candidate genes (69 from our library and 191 from OneArray™) that were differentially expressed in the CC of pregnant patients compared to the non-pregnant group. Conversely, the second group contains 231 potential incompetence markers (29 in our library and 202 from OneArray™) that were downexpressed in the CC of pregnant patients compared to the non pregnant. These candidates are potential negative indicators of oocyte quality.

It is to note that two positive markers of competence (overexpressed candidates) were common between our library and the OneArray™: HIST1H4C and GSDMA. These selected candidates were then ordered according to their redundancy in different libraries, their signal intensities and their recurrence inside the same library.

Among both overexpressed and underexpressed candidates provided by the hybridization on our custom-made library, there are some clone transcript sequences that don't match significantly with any known transcript sequence on the NCBI data base. These still unidentified candidates were put in both overexpressed and downexpressed candidate lists produced from our custom-made hybridizations.

### Real Time PCR Analysis:

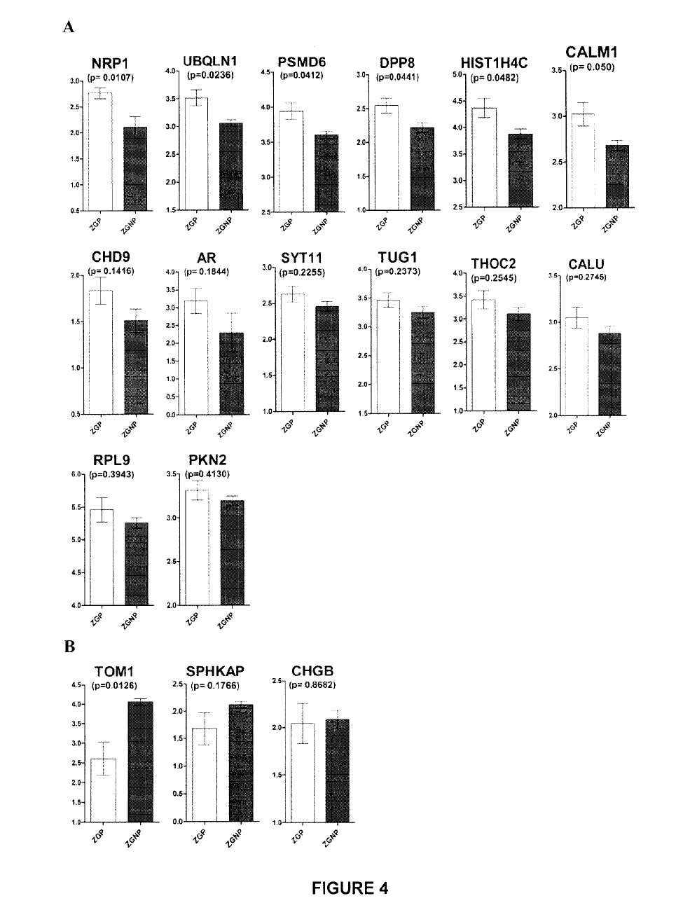

In order to validate our both positive and negative makers lists, 17 candidates were chosen for additional validation by quantitative real time PCR. The QPCR validation was achieved on the CC tissues of the two ZGP and ZGNP groups (positive and negative groups). Among the 17 selected genes, six positive markers of oocyte quality and successful pregnancy were statistically significant between pregnant and non-pregnant patient groups. These candidates are DPP8 (p=0.0441), HIST1H4C (p=0.0482), UBQLN1 (p=0.0236), CALM1 (p=0.05), NRP1 (ₚ=0.0107) and PSMD6 (p=0.0412) (FIGS. 4A and 4B)

Among the three (3) downexpressed markers assessed, TOM1(p=0.0126) was confirmed as negative marker differentially expressed in the CC of the non pregnant patient group. Concerning SPHKAP (p=0.1766) and CHGB (p=0.8682), they were not significant. TOM1 is therefore an interesting candidate was highly significant following the QPCR validation (p=0.0126) (FIG. 4A).

Other overexpressed candidate genes were not statistically significant including CALU (p=0.2745), PKN2 (p=0.413), RPL9 (p=0.3943), SYT11 (p=0.2255), THOC2 (p=0.2545), CHD9 (p=0.1416), AR (p=0.1844) and TUG1 (p=0.2373). These candidates remain potential positive markers and require validation with different tissues and a large number of patients (FIGS. 4A, and 4B).

These CC candidates were selected using two different platforms. The first is a custom-made microarrays platform obtained by Suppressive subtractive hybridizations of cDNA sequences, whereas the second one is the OneArray™ commercial arrays. These in vivo markers reflect the normal physiological and genomic contexts needed for good oocyte production and successful pregnancy. The proximity of the oocyte confer CC a high potential to notify its developmental potential both in ICSI programs or IVF cycles. They represent a valuable tool in clinical aspect not only in the selection of good quality oocyte that leads to successful pregnancy and healthy embryo, but also to assess efficiency and optimize the of the used superovulation protocols. They could be used also to optimize the culture media used during in vitro maturation protocols. The level expression of these positive and negative markers in CC collected following IVM or a superovulation protocol should correlate with those find in successful pregnancy context find in the in vivo context and reported herein.

Table 5 hereinafter provides a list of overexpressed candidates (69) of the hybridization on our custom-made library with their fold change.

Table 6 hereinafter provides a list of overexpressed candidates (191) of the hybridization on the OneArray™ library with their fold change.

Table 7 hereinafter provides a list of downexpressed candidates (29) of the hybridization on our custom-made library with their fold change.

Table 8 hereinafter provides a list of downexpressed candidates (202) of the hybridization on the OneArray™ library with their fold change.

TABLE 5
Overexpressed candidates (69) of the hybridization on our custom-made library with their fold change

| | | Official | GenBank | Unigene | Fold |
|---|---|---|---|---|---|
| Order | Gene full name | symbol | accession | accession | change |
| 1 | Homo sapiens ubiquilin 1 | UBQLN1 | BC017289 | Hs.9589 | 11.05 |
| 2 | Homo sapiens protein phosphatase 1G (formerly 2C), magnesium-dependent, | PPM1G | NM_002707.3 | Hs.643951 | 11.04 |
| | gamma isoform | | | | |
| 3 | Homo sapiens ribosomal protein L9, mRNA | RPL9 | BC066318 | Hs.719072 | 8.54 |
| 4 | Homo sapiens proteasome (prosome, macropain) 26S subunit, non-ATPase, | PSMD6 | BC000630 | Hs.152536 | 8.45 |
| 5 | Homo sapiens gametogenetin binding protein 2 | GGNBP2 | NM_024835 | Hs.514116 | 7.88 |
| 6 | Homo sapiens histone cluster 1, H4c | HIST1H4C | NM_003542 | Hs.46423 | 6.68 |
| 7 | PREDICTED: Homo sapiens hypothetical protein LOC100127894 | LOC100127894 | XM_001723874.1 | GeneID: | 6.15 |
| | | | | 100127894 | |
| 8 | Homo sapiens LIM domain 7 | LMO7 | NM_005358.5 | Hs.207631 | 5.387 |
| 9 | Homo sapiens proline-rich transmembrane protein 2 | PRRT2 | BC053594 | Hs.655071 | 4.72 |
| 10 | Homo sapiens gasdermin A | GSDMA | NM_178171 | Hs.448873 | 4.37 |
| 11 | Homo sapiens iduronidase, alpha-L- | IDUA | NM_000203.3 | Hs.89560 | 4.13 |
| 12 | Homo sapiens PC4 and SFRS1 interacting protein 1 | PSIP1 | BC033817 | Hs.658434 | 4.08 |
| 13 | Homo sapiens THO complex 2 (THOC2), transcript variant 1 | THOC2 | NM_001081550.1 | Hs.592243 | 3.90 |
| 14 | Homo sapiens calumein | CALU | AF013759 | Hs.7753 | 3.89 |
| 15 | Homo sapiens internexin neuronal intermediate filament protein, alpha | INA | NM_032727.3 | Hs.500916 | 3.80 |
| 16 | Homo sapiens eukaryotic translation initiation factor 4 gamma, | EIF4G3 | BC094683 | Hs.467084 | 3.72 |
| 17 | Homo sapiens Cell division cycle and apoptosis regulator protein 1 | CCAR1 | BC026036 | Hs.49853 | 3.64 |
| 18 | Homo sapiens calmodulin 1 (phosphorylase kinase, delta) | CALM1 | BC007965 | Hs.282410 | 3.63 |
| 19 | Homo sapiens COX2 cytochrome c oxidase subunit II | MT-CO2 | BC021246 | X15759 | 3.59 |
| 20 | Homo sapiens general transcription factor IIIC, polypeptide 1, alpha 220 kDa | GTF3C1 | NM_001520.3 | Hs.371718 | 3.59 |
| 21 | Homo sapiens androgen receptor | AR | NM_000044.2 | Hs.496240 | 3.33 |
| 22 | Homo sapiens forkhead box K1 | FOXK1 | NM_001037165 | Hs.708095 | 3.32 |
| 23 | Homo sapiens RAN, member RAS oncogene family | RAN | BC072000 | Hs.10842 | 3.05 |
| 24 | Homo sapiens carnitine palmitoyltransferase 1A (liver) | CPT1A | NM_001876.3 | Hs.503043 | 3.02 |
| 25 | Homo sapiens neuropilin 1 | NRP1 | AB209641 | Hs.131704 | 2.98 |
| 26 | Homo sapiens retinol dehydrogenase 11 | RDH11 | NM_016026 | Hs.226007 | 2.92 |
| 27 | Homo sapiens prolactin receptor | PRLR | NM_000949.4 | Hs.368587 | 2.88 |
| 28 | Homo sapiens iduronidase, alpha-L- | IDUA | NM_000203.3 | Hs.89560 | 2.88 |
| 29 | Homo sapiens minichromosome maintenance complex component 3 | MCM3AP | BC104958.1 | Hs.389037 | 2.82 |
| | associated protein | | | | |
| 30 | Homo sapiens synaptotagmin XI | SYT11 | BC039205 | Hs.32984 | 2.76 |
| 31 | Homo sapiens BCDIN3 domain containing | BCDIN3D | NM_181708.2 | Hs.142736 | 2.76 |
| 32 | Homo sapiens membrane bound O-acyltransferase domain containing 1 | MBOAT1 | NM_001080480 | Hs.377830 | 2.76 |
| 33 | Homo sapiens DEAD (Asp-Glu-Ala-Asp) box polypeptide 51 | DDX51 | BC040185 | Hs.445168 | 2.75 |
| 34 | Homo sapiens Rho-related BTB domain containing 3 | RHOBTB3 | BC041337 | Hs.445030 | 2.69 |
| 35 | Homo sapiens ribosomal protein S4, X-linked | RPS4X | NM_001007.4 | Hs.118076 | 2.56 |
| 36 | Homo sapiens cyclin-dependent kinase inhibitor 1A (p21, Cip1) | CDKN1A | NM_000389.3 | Hs.370771 | 2.55 |
| 37 | Homo sapiens protein phosphatase 1B (formerly 2C), magnesium-dependent, | PPM1B | NM_177968.2 | Hs.416769 | 2.51 |
| | beta isoform | | | | |
| 38 | Homo sapiens vacuolar protein sorting 16 homolog (S. cerevisiae) | VPS16 | NM_022575.2 | Hs.269577 | 2.445 |
| 39 | Homo sapiens hook homolog 3 (Drosophila) | HOOK3 | NM_032410.3 | Hs.162852 | 2.437 |
| 40 | Homo sapiens GABA(A) receptor-associated protein | GABARAP | NM_007278.1 | Hs.647421 | 2.403 |
| 41 | Homo sapiens FK506 binding protein 11, 19 kDa | FKBP11 | NM_016594.2 | Hs.655103 | 2.4 |
| 42 | Homo sapiens toll interacting protein | TOLLIP | NM_019009.2 | Hs.368527 | 2.30 |
| 43 | Homo sapiens family with sequence similarity 8, member A1 | FAM8A1 | NM_016255 | Hs.95260 | 2.18 |
| 44 | Homo sapiens proteasome maturation protein | POMP | BC003390 | Hs.268742 | 2.11 |
| 45 | Homo sapiens POTE ankyrin domain family, member F | POTEF | NM_001099771.2 | Hs.580547 | 2.07 |
| 46 | Homo sapiens TP53 regulating kinase | TP53RK | BC035461 | Hs.440263 | 2.06 |
| 47 | Homo sapiens Caspase 9, apoptosis-related cysteine peptidase | CASP9 | AY732490 | Hs.329502 | 2.05 |
| 48 | Homo sapiens methyltransferase like 5 | METTL5 | BC093014 | Hs.470553 | 2.04 |
| 49 | Homo sapiens SEC14-like 4 (S. cerevisiae) | SEC14L4 | NM_174977 | Hs.517541 | 2.02 |
| 50 | Homo sapiens nitrilase family, member 2 | NIT2 | AF284574 | Hs.439152 | 2.02 |
| 51 | Homo sapiens peroxiredoxin 2 | PRDX2 | BC000452 | Hs.432121 | 2.01 |

| No. | Unkown transcripts' sequences | length (pb) | Fold change |
|---|---|---|---|
| 1 | SEQ ID NO: 88 | 250 | 11 |
| 2 | SEQ ID NO: 89 | 418 | 6.1 |
| 3 | SEQ ID NO: 90 | 327 | 4.7 |
| 4 | SEQ ID NO: 91 | 245 | 4.1 |
| 5 | SEQ ID NO: 92 | 602 | 4 |
| 6 | SEQ ID NO: 93 | 402 | 3.8 |
| 7 | SEQ ID NO: 94 | 464 | 3.3 |
| 8 | SEQ ID NO: 95 | 372 | 2.8 |
| 9 | SEQ ID NO: 96 | 367 | 2.88 |
| 10 | SEQ ID NO: 97 | 462 | 2.82 |
| 11 | SEQ ID NO: 98 | 650 | 2.7 |
| 12 | SEQ ID NO: 99 | 459 | 2.3 |
| 13 | SEQ ID NO: 100 | 488 | 2.1 |
| 14 | SEQ ID NO: 101 | 473 | 2 |
| 15 | SEQ ID NO: 102 | 448 | 2 |
| 16 | SEQ ID NO: 103 | 519 | 5.4 |
| 17 | SEQ ID NO: 104 | 326 | 3.6 |
| 18 | SEQ ID NO: 105 | 273 | 3.3 |

TABLE 6
Overexpressed candidates (191) of the hybridization on the OneArray library with their fold change

| | | Official | Genbank | Unigene | Fold |
|---|---|---|---|---|---|
| No. | Gene full name | symbol | accession | accession | change |
| 1 | Homo sapiens chromodomain helicase DNA binding protein 9 | CHD9 | NM_025134.4 | Hs.622347 | 30.77 |
| 2 | Homo sapiens chromosome 12 open reading frame 30 | C12orf30 | NM_024953.2 | Hs.530941 | 29.09 |
| 3 | Homo sapiens histone cluster 1, H4c | HIST1H4C | NM_003542.3 | Hs.46423 | 21.50 |
| 4 | Homo sapiens additional sex combs like 2 (Drosophila) | ASXL2 | NM_018263.4 | Hs.594386 | 20.84 |
| 5 | Homo sapiens eukaryotic translation initiation factor 2, subunit 1 alpha, | EIF2S1 | NM_004094.4 | Hs.151777 | 20.31 |
| | 35 kDa | | | | |
| 6 | Homo sapiens protein kinase N2 | PKN2 | NM_006256.2 | Hs.440833 | 18.45 |
| 7 | Homo sapiens taurine upregulated 1 (non-protein coding) | TUG1 | NR_002323.1 | Hs.554829 | 17.10 |
| 8 | Homo sapiens ubiquitin specific peptidase 4 | USP4 | NM_003363.3 | Hs.77500 | 16.87 |
| 9 | Homo sapiens CXXC finger 5 | CXXC5 | NM_016463.7 | Hs.189119 | 15.74 |
| 10 | Homo sapiens transmembrane and coiled-coil domain family 1 | TMCC1 | NM_001128224.1 | Hs.477547 | 14.38 |
| 11 | Homo sapiens Rap guanine nucleotide exchange factor (GEF) 6 | RAPGEF6 | NM_016340.4 | Hs.483329 | 14.22 |
| 12 | Homo sapiens asparaginyl-tRNA synthetase | NARS | NM_004539.3 | Hs.465224 | 14.13 |
| 13 | Homo sapiens bobby sox homolog (Drosophila) | BBX | NM_001142568.1 | Hs.124366 | 14.05 |
| 14 | Homo sapiens poliovirus receptor-related 3 | PVRL3 | NM_015480.1 | Hs.293917 | 13.97 |
| 15 | Homo sapiens H3 histone, family 3A | H3F3A | NM_002107.3 | Hs.546259 | 13.76 |
| 16 | Homo sapiens apolipoprotein L, 6 | APOL6 | NM_030641.3 | Hs.257352 | 13.37 |
| 17 | Homo sapiens histone cluster 1, H4l | HIST1H4L | NM_003546.2 | Hs.533295 | 13.14 |
| 18 | Homo sapiens phosphoglycolate phosphatase | PGP | NM_001042371.2 | Hs.442634 | 12.74 |
| 19 | Homo sapiens sorbin and SH3 domain containing 2 | SORBS2 | BC011883 | Hs.655143 | 12.66 |
| 20 | Homo sapiens dipeptidyl-peptidase 8 | DPP8 | NM_197960.2 | Hs.591106 | 12.09 |
| 21 | Homo sapiens zinc finger, BED-type containing 3 | ZBED3 | NM_032367.2 | Hs.584988 | 11.97 |
| 22 | Homo sapiens chromosome 17 open reading frame 63 | C17orf63 | NM_018182.2 | Hs.564533 | 11.10 |
| 23 | Homo sapiens suppressor of variegation 4-20 homolog 1 (Drosophila) | SUV420H1 | NM_017635.3 | Hs.632120 | 10.69 |
| 24 | Homo sapiens KIAA2026 | KIAA2026 | NM_001017969.2 | Hs.535060 | 10.52 |
| 25 | Homo sapiens androgen receptor | AR | NM_000044.2 | Hs.496240 | 10.37 |
| 26 | Homo sapiens hyperpolarization activated cyclic nucleotide-gated | HCN4 | NM_005477.2 | Hs.86941 | 10.12 |
| | potassium channel 4 | | | | |
| 27 | Homo sapiens ataxin 2 | ATXN2 | NM_002973.3 | Hs.76253 | 10.00 |
| 28 | Homo sapiens speckle-type POZ protein | SPOP | NM_001007226.1 | Hs.718421 | 9.99 |
| 29 | Homo sapiens mediator complex subunit 9 | MED9 | NM_018019.2 | Hs.244595 | 9.91 |
| 30 | Homo sapiens histone cluster 1, H4h | HIST1H4H | NM_003543.3 | Hs.591790 | 9.58 |
| 31 | Homo sapiens DnaJ (Hsp40) homolog, subfamily C, member 30 | DNAJC30 | NM_032317.2 | Hs.647046 | 9.43 |
| 32 | Homo sapiens LYR motif containing 2 | LYRM2 | NM_020466.4 | Hs.177275 | 8.99 |
| 33 | Homo sapiens protein kinase, AMP-activated, alpha 1 catalytic subunit | PRKAA1 | NM_206907.3 | Hs.43322 | 8.98 |
| 34 | Homo sapiens adenosylhomocysteinase-like 1 | AHCYL1 | NM_006621.4 | Hs.705418 | 8.70 |
| 35 | Homo sapiens chromosome 6 open reading frame 72 | C6orf72 | NM_138785.2 | Hs.438872 | 8.65 |
| 36 | Homo sapiens general transcription factor IIH, polypeptide 5 | GTF2H5 | NM_207118.2 | Hs.356224 | 8.36 |
| 37 | Homo sapiens survival of motor neuron 1, telomeric | SMN1 | NM_000344.3 | Hs.535788 | 8.31 |
| 38 | Homo sapiens apolipoprotein B mRNA editing enzyme, catalytic | APOBEC3D | NM_152426.3 | Hs.658626 | 8.28 |
| | polypeptide-like 3D | | | | |
| 39 | Homo sapiens cytochrome c oxidase subunit VIb polypeptide 2 (testis) | COX6B2 | NM_144613.4 | Hs.550544 | 7.96 |
| 40 | Homo sapiens protocadherin beta 19 pseudogene | PCDHB19P | NR_001282.2 | Hs.570898 | 7.94 |
| 41 | Homo sapiens leucine-rich repeat kinase 1 | LRRK1 | NM_024652.3 | Hs.407918 | 7.90 |
| 42 | Homo sapiens actinin, alpha 4 | ACTN4 | NM_004924.3 | Hs.270291 | 7.85 |
| 43 | Homo sapiens RAB5B, member RAS oncogene family | RAB5B | NM_002868.2 | Hs.567328 | 7.66 |
| 44 | Homo sapiens syntrophin, beta 2 (dystrophin-associated protein A1, | SNTB2 | NM_006750.3 | Hs.461117 | 7.54 |
| | 59 kDa, basic component 2) | | | | |
| 45 | Homo sapiens dynamin 1-like | DNM1L | NM_012062.3 | Hs.556296 | 7.45 |
| 46 | Homo sapiens poly (ADP-ribose) polymerase family, member 14 | PARP14 | NM_017554.2 | Hs.518203 | 7.43 |
| 47 | Homo sapiens apolipoprotein L, 2 | APOL2 | NM_145637.1 | Hs.474740 | 7.40 |
| 48 | Homo sapiens motile sperm domain containing 2 | MOSPD2 | NM_152581.2 | Hs.715564 | 7.36 |
| 49 | Homo sapiens SHC SH2-domain binding protein 1 | SHCBP1 | NM_024745.4 | Hs.123253 | 7.34 |
| 50 | Homo sapiens prolyl 4-hydroxylase, beta polypeptide | P4HB | NM_000918.3 | Hs.464336 | 7.30 |
| 51 | Homo sapiens bone morphogenetic protein 8b | BMP8B | NM_001720.3 | Hs.664022 | 7.30 |
| 52 | Homo sapiens chromosome 21 open reading frame 45 | C21orf45 | NM_018944.2 | Hs.190518 | 7.30 |
| 53 | Homo sapiens proteasome (prosome, macropain) subunit, alpha type, 7 | PSMA7 | NM_002792.2 | Hs.233952 | 7.25 |
| 54 | Homo sapiens golgi autoantigen, golgin subfamily a-like | LOC283767 | NM_001001413.3 | Hs.531569 | 7.19 |
| 55 | Homo sapiens zinc finger protein 668 | ZNF668 | NM_024706.3 | Hs.102928 | 6.99 |
| 56 | Homo sapiens ubiquitin specific peptidase 47 | USP47 | NM_017944.3 | Hs.577256 | 6.99 |
| 57 | Homo sapiens 5-methyltetrahydrofolate-homocysteine methyltransferase | MTRR | NM_024010.2 | Hs.481551 | 6.86 |
| | reductase | | | | |
| 58 | Homo sapiens reticulon 4 | RTN4 | NM_020532.4 | Hs.704007 | 6.85 |
| 59 | Homo sapiens LRRN4 C-terminal like | LRRN4CL | NM_203422.1 | Hs.427449 | 6.73 |
| 60 | Homo sapiens ring finger protein 125 | RNF125 | NM_017831.3 | Hs.633703 | 6.67 |
| 61 | Homo sapiens centrosomal protein 135 kDa | CEP135 | NM_025009.3 | Hs.518767 | 6.65 |
| 62 | Homo sapiens nuclear receptor coactivator 1 | NCOA1 | NM_003743.4 | Hs.596314 | 6.61 |
| 63 | Homo sapiens nudE nuclear distribution gene E homolog 1 | NDE1 | NM_001143979.1 | Hs.655378 | 6.56 |
| 64 | Homo sapiens dual specificity phosphatase 8 | DUSP8 | NM_004420.2 | Hs.41688 | 6.54 |
| 65 | Homo sapiens asparagine-linked glycosylation 1, beta-1,4- | ALG1 | NM_019109.4 | Hs.592086 | 6.51 |
| | mannosyltransferase homolog (S. cerevisiae) | | | | |
| 66 | Homo sapiens frizzled homolog 3 (Drosophila) | FZD3 | NM_017412.2 | Hs.40735 | 6.45 |
| 67 | Homo sapiens tubulin folding cofactor D | TBCD | NM_005993.4 | Hs.464391 | 6.41 |
| 68 | PREDICTED: Homo sapiens hypothetical protein LOC100287862 | LOC100287862 | XM_002342250.1 | GeneID: | 6.39 |
| | | | | 100287862 | |
| 69 | Homo sapiens centromere protein N | CENPN | NM_001100624.1 | Hs.55028 | 6.20 |
| 70 | Homo sapiens zinc finger protein 492 | ZNF492 | NM_020855.2 | Hs.232108 | 6.19 |
| 71 | Homo sapiens collagen, type IX, alpha 1 | COL9A1 | NM_001851.4 | Hs.590892 | 6.12 |
| 72 | Homo sapiens neuroblastoma breakpoint family, member 10 | NBPF10 | NM_001039703.3 | Hs.515947 | 6.05 |
| 73 | Homo sapiens family with sequence similarity 74, member A1 | FAM74A1 | NR_026803.1 | Hs.553802 | 6.02 |
| 74 | Homo sapiens Rho-related BTB domain containing 3 | RHOBTB3 | NM_014899.3 | Hs.445030 | 6.01 |
| 75 | Homo sapiens WD repeat domain 82 | WDR82 | NM_025222.3 | Hs.194110 | 5.92 |
| 76 | Homo sapiens solute carrier family 38, member 1 | SLC38A1 | NM_030674.3 | Hs.694701 | 5.86 |
| 77 | Homo sapiens zinc finger, RAN-binding domain containing 1 | ZRANB1 | NM_017580.2 | Hs.595158 | 5.86 |
| 78 | Homo sapiens methyltransferase 5 domain containing 1 | METT5D1 | NM_152636.2 | Hs.243326 | 5.77 |
| 79 | Homo sapiens lysophosphatidic acid receptor 5 | LPAR5 | NM_020400.5 | Hs.155538 | 5.66 |
| 80 | Homo sapiens bone morphogenetic protein 7 | BMP7 | NM_001719.2 | Hs.473163 | 5.61 |
| 81 | Homo sapiens tryptase alpha/beta 1 | TPSAB1 | NM_003294.3 | Hs.405479 | 5.40 |
| 82 | Homo sapiens THUMP domain containing 1 | THUMPD1 | NM_017736.3 | Hs.460232 | 5.35 |
| 83 | Homo sapiens gasdermin A | GSDMA | NM_178171.4 | Hs.448873 | 5.29 |
| 84 | Homo sapiens muscleblind-like (Drosophila) | MBNL1 | NM_021038.3 | Hs.478000 | 5.29 |
| 85 | Homo sapiens DC-STAMP domain containing 2 | DCST2 | NM_144622.2 | Hs.591491 | 5.16 |
| 86 | phosphatidic acid phosphatase type 2 domain containing 2 | PPAPDC2 | NM_203453.2 | Hs.107510 | 5.12 |
| 87 | Homo sapiens interleukin 17 receptor D | IL17RD | NM_017563.3 | Hs.150725 | 5.09 |
| 88 | Homo sapiens SWI/SNF related, matrix associated, actin dependent | SMARCC2 | NM_001130420.1 | Hs.236030 | 5.08 |
| | regulator of chromatin, subfamily c, member 2 | | | | |
| 89 | Homo sapiens enabled homolog (Drosophila) | ENAH | NM_001008493.1 | Hs.497893 | 4.95 |
| 90 | Homo sapiens histone cluster 1, H4b | HIST1H4B | NM_003544.2 | Hs.143080 | 4.91 |
| 91 | Homo sapiens latent transforming growth factor beta binding protein 3 | LTBP3 | NM_001130144.2 | Hs.289019 | 4.89 |
| 92 | Homo sapiens zinc finger protein 658 | ZNF658 | NM_033160.5 | Hs.522147 | 4.84 |
| 93 | Homo sapiens ubiquitin specific peptidase 9, X-linked | USP9X | NM_001039591.2 | Hs.77578 | 4.82 |
| 94 | Homo sapiens nuclear pore complex interacting protein-like 3 | NPIPL3 | NM_130464.1 | Hs.552700 | 4.79 |
| 95 | Homo sapiens zinc finger protein 36, C3H type-like 1 | ZFP36L1 | NM_004926.2 | Hs.85155 | 4.77 |
| 96 | Homo sapiens trinucleotide repeat containing 6B | TNRC6B | NM_001024843.1 | Hs.372082 | 4.74 |
| 97 | Homo sapiens high-mobility group box 1 | HMGB1 | NM_002128.4 | Hs.596078 | 4.66 |
| 98 | Homo sapiens single-stranded DNA binding protein 1 | SSBP1 | NM_003143.1 | Hs.490394 | 4.61 |
| 99 | Homo sapiens pleckstrin homology domain containing, family O member 1 | PLEKHO1 | NM_016274.4 | Hs.438824 | 4.56 |
| 100 | PREDICTED: Homo sapiens hypothetical protein LOC100291631 | LOC100291631 | XM_002344465.1 | GeneID: | 4.55 |
| | | | | 100291631 | |
| 101 | Homo sapiens mediator complex subunit 1 | MED1 | NM_004774.3 | Hs.643754 | 4.48 |
| 102 | Homo sapiens ribonuclease P/MRP 30 kDa subunit | RPP30 | NM_006413.4 | Hs.139120 | 4.44 |
| 103 | Homo sapiens jub, ajuba homolog (Xenopus laevis) | JUB | NM_032876.4 | Hs.655832 | 4.43 |
| 104 | Homo sapiens interleukin 17 receptor A | IL17RA | NM_014339.4 | Hs.129751 | 4.43 |
| 105 | Homo sapiens mannosidase, alpha, class 2A, member 1 | MAN2A1 | NM_002372.2 | Hs.432822 | 4.40 |
| 106 | Homo sapiens transmembrane protein 181 | TMEM181 | NM_020823.1 | Hs.99145 | 4.36 |
| 107 | Homo sapiens MYST histone acetyltransferase (monocytic leukemia) 3 | MYST3 | NM_001099413.1 | Hs.491577 | 4.32 |
| 108 | Homo sapiens zinc finger and BTB domain containing 16 | ZBTB16 | NM_006006.4 | Hs.591945 | 4.26 |
| 109 | Homo sapiens regulator of chromosome condensation (RCC1) and BTB | RCBTB1 | NM_018191.3 | Hs.508021 | 4.25 |
| | (POZ) domain containing protein 1 | | | | |
| 110 | Homo sapiens actin related protein 2/3 complex, subunit 2, 34 kDa | ARPC2 | NM_152862.1 | Hs.529303 | 4.23 |
| 111 | Homo sapiens NUAK family, SNF1-like kinase, 1 | NUAK1 | NM_014840.2 | Hs.719171 | 4.22 |
| 112 | Homo sapiens potassium channel tetramerisation domain containing 1 | KCTD1 | NM_001142730.1 | Hs.526630 | 4.15 |
| 113 | Homo sapiens hypothetical protein DKFZp586I1420 | DKFZP586I1420 | NR_002186.1 | GeneID: | 4.12 |
| | | | | 222161 | |
| 114 | Homo sapiens nuclear pore complex interacting protein-like 3 | NPIPL3 | NM_130464.1 | Hs.552700 | 4.04 |
| 115 | Homo sapiens F-box protein 44 | FBXO44 | NM_001014765.1 | Hs.556006 | 4.03 |
| 116 | Homo sapiens ubinuclein 1 | UBN1 | NM_001079514.1 | Hs.440219 | 3.94 |
| 117 | Homo sapiens family with sequence similarity 59, member A | FAM59A | NM_022751.1 | Hs.444314 | 3.91 |
| 118 | Homo sapiens integrin, beta 3 (platelet glycoprotein IIIa, antigen CD61) | ITGB3 | NM_000212.2 | Hs.218040 | 3.90 |
| 119 | Homo sapiens DEAD (Asp-Glu-Ala-Asp) box polypeptide 58 | DDX58 | NM_014314.3 | Hs.190622 | 3.85 |
| 120 | Homo sapiens chromosome 4 open reading frame 34 | C4orf34 | NM_174921.1 | Hs.576320 | 3.85 |
| 121 | Homo sapiens phospholipase C, beta 2 | PLCB2 | NM_004573.2 | Hs.355888 | 3.84 |
| 122 | Homo sapiens potassium voltage-gated channel, subfamily G, member 1 | KCNG1 | NM_002237.3 | Hs.118695 | 3.83 |
| 123 | Homo sapiens dehydrogenase E1 and transketolase domain containing 1 | DHTKD1 | NM_018706.5 | Hs.104980 | 3.81 |
| 124 | Homo sapiens leucine rich repeat and Ig domain containing 2 | LINGO2 | NM_152570.1 | Hs.715650 | 3.77 |
| 125 | Homo sapiens ral guanine nucleotide dissociation stimulator-like 1 | RGL1 | NM_015149.3 | Hs.497148 | 3.75 |
| 126 | Homo sapiens kinetochore associated 1 | KNTC1 | NM_014708.4 | Hs.300559 | 3.73 |
| 127 | Homo sapiens ubiquitin specific peptidase 42 | USP42 | NM_032172.2 | Hs.31856 | 3.72 |
| 128 | Homo sapiens zinc finger protein 833 | ZNF833 | NM_001013691.2 | Hs.672384 | 3.69 |
| 129 | Homo sapiens coatomer protein complex, subunit alpha | COPA | NM_001098398.1 | Hs.162121 | 3.65 |
| 130 | Homo sapiens Ly1 antibody reactive homolog (mouse) | LYAR | NM_017816.2 | Hs.425427 | 3.42 |
| 131 | Homo sapiens isocitrate dehydrogenase 1 (NADP+), soluble | IDH1 | NM_005896.2 | Hs.593422 | 3.42 |
| 132 | Homo sapiens MAP-kinase activating death domain | MADD | NM_003682.3 | Hs.82548 | 3.42 |
| 133 | Homo sapiens solute carrier family 1 (glial high affinity glutamate | SLC1A2 | NM_004171.3 | Hs.502338 | 3.39 |
| | transporter), member 2 | | | | |
| 134 | Homo sapiens family with sequence similarity 171, member A1 | FAM171A1 | NM_001010924.1 | Hs.66762 | 3.38 |
| 135 | Homo sapiens olfactory receptor, family 51, subfamily E, member 2 | OR51E2 | NM_030774.3 | Hs.501758 | 3.36 |
| 136 | Homo sapiens unc-13 homolog D (C. elegans) | UNC13D | NM_199242.2 | Hs.41045 | 3.31 |
| 137 | Homo sapiens histone cluster 1, H4f | HIST1H4F | NM_003540.3 | Hs.247816 | 3.31 |
| 138 | Homo sapiens COX19 cytochrome c oxidase assembly homolog (S. | COX19 | NM_001031617.2 | Hs.121593 | 3.30 |
| | cerevisiae) | | | | |
| 139 | Homo sapiens src kinase associated phosphoprotein 2 | SKAP2 | NM_003930.3 | Hs.200770 | 3.30 |
| 140 | Homo sapiens RWD domain containing 1 | RWDD1 | NM_015952.2 | Hs.532164 | 3.16 |
| 141 | Homo sapiens ryanodine receptor 2 (cardiac) | RYR2 | NM_001035.2 | Hs.109514 | 3.10 |
| 142 | Homo sapiens zinc finger protein 492 | ZNF492 | NM_020855.2 | Hs.232108 | 3.09 |
| 143 | Homo sapiens zinc finger, DHHC-type containing 3 | ZDHHC3 | NM_016598.2 | Hs.61430 | 3.05 |
| 144 | Homo sapiens transmembrane protein 151B | TMEM151B | NM_001137560.1 | Hs.632851 | 3.00 |
| 145 | Homo sapiens oncoprotein induced transcript 3 | OIT3 | NM_152635.1 | Hs.8366 | 2.98 |
| 146 | Homo sapiens stromal antigen 2 | STAG2 | NM_001042749.1 | Hs.496710 | 2.94 |
| 147 | Homo sapiens family with sequence similarity 129, member A | FAM129A | NM_052966.2 | Hs.518662 | 2.93 |
| 148 | Homo sapiens ribonuclease, RNase A family, 11 (non-active) | RNASE11 | NM_145250.3 | Hs.112761 | 2.91 |
| 149 | Homo sapiens zinc finger, SWIM-type containing 4 | ZSWIM4 | NM_023072.2 | Hs.466015 | 2.90 |
| 150 | Homo sapiens G protein-coupled receptor kinase 4 | GRK4 | NM_182982.2 | Hs.32959 | 2.88 |
| 151 | omo sapiens aryl hydrocarbon receptor interacting protein | AIP | NM_003977.2 | Hs.412433 | 2.88 |
| 152 | Homo sapiens Yip1 domain family, member 5 | YIPF5 | NM_001024947.2 | Hs.372050 | 2.87 |
| 153 | Homo sapiens Charcot-Leyden crystal protein | CLC | NM_001828.4 | Hs.889 | 2.84 |
| 154 | Homo sapiens delta-like 3 (Drosophila) | DLL3 | NM_016941.3 | Hs.127792 | 2.77 |
| 155 | Homo sapiens SMAD family member 5 | SMAD5 | NM_001001419.1 | Hs.167700 | 2.75 |
| 156 | Homo sapiens RAB8A, member RAS oncogene family | RAB8A | NM_005370.4 | Hs.642874 | 2.72 |
| 157 | Homo sapiens EPH receptor B1 | EPHB1 | NM_004441.3 | Hs.116092 | 2.72 |
| 158 | Homo sapiens vinculin | VCL | NM_014000.2 | Hs.643896 | 2.71 |
| 159 | Homo sapiens DEAD (Asp-Glu-Ala-Asp) box polypeptide 21 | DDX21 | NM_004728.2 | Hs.223141 | 2.61 |
| 160 | Homo sapiens heterogeneous nuclear ribonucleoprotein R | HNRNPR | NM_005826.3 | Hs.373763 | 2.61 |
| 161 | Homo sapiens TGFB-induced factor homeobox 2 | TGIF2 | NM_021809.5 | Hs.632264 | 2.58 |
| 162 | Homo sapiens keratin associated protein 20-1 | KRTAP20-1 | NM_181615.1 | Hs.553697 | 2.57 |
| 163 | Homo sapiens ribosomal protein S27-like | RPS27L | NM_015920.3 | Hs.108957 | 2.56 |
| 164 | Homo sapiens nardilysin (N-arginine dibasic convertase) | NRD1 | NM_002525.2 | Hs.584782 | 2.50 |
| 165 | Homo sapiens late cornified envelope 1C | LCE1C | NM_178351.3 | Hs.516429 | 2.47 |
| 166 | Homo sapiens arginine vasopressin receptor 2 | AVPR2 | NM_000054.4 | Hs.567240 | 2.45 |
| 167 | Homo sapiens histone cluster 1, H4e | HIST1H4E | NM_003545.3 | Hs.662174 | 2.45 |
| 168 | Homo sapiens ubiquinol-cytochrome c reductase hinge protein-like | UQCRHL | NM_001089591.1 | Hs.568229 | 2.44 |
| 169 | Homo sapiens musashi homolog 1 (Drosophila) | MSI1 | NM_002442.2 | Hs.158311 | 2.44 |
| 170 | Homo sapiens chromosome 21 open reading frame 63 | C21orf63 | NM_058187.3 | Hs.208358 | 2.42 |
| 171 | Homo sapiens HLA-B associated transcript 2-like | BAT2L | NM_013318.3 | Hs.495349 | 2.39 |
| 172 | Homo sapiens BCL2-associated athanogene 3 | BAG3 | NM_004281.3 | Hs.523309 | 2.38 |
| 173 | Homo sapiens dipeptidyl-peptidase 9 | DPP9 | NM_139159.4 | Hs.515081 | 2.35 |
| 174 | Homo sapiens transmembrane and ubiquitin-like domain containing 2 | TMUB2 | NM_001076674.1 | Hs.181391 | 2.34 |
| 175 | Homo sapiens heterogeneous nuclear ribonucleoprotein U (scaffold | HNRNPU | NM_031844.2 | Hs.106212 | 2.32 |
| | attachment factor A) | | | | |
| 176 | Homo sapiens APEX nuclease (apurinic/apyrimidinic endonuclease) 2 | APEX2 | NM_014481.2 | Hs.659558 | 2.31 |
| 177 | Homo sapiens adhesion regulating molecule 1 | ADRM1 | NM_175573.1 | Hs.90107 | 2.31 |
| 178 | Homo sapiens zinc finger protein 275 | ZNF275 | NM_001080485.2 | Hs.348963 | 2.30 |
| 179 | Homo sapiens serine palmitoyltransferase, long chain base subunit 2 | SPTLC2 | NM_004863.2 | Hs.435661 | 2.29 |
| 180 | Homo sapiens coatomer protein complex, subunit alpha | COPA | NM_004371.3 | Hs.162121 | 2.28 |
| 181 | Homo sapiens GATA like protein-1 | GLP-1 | NM_001103167.1 | Hs.709296 | 2.25 |
| 182 | Homo sapiens transforming growth factor, beta receptor III | TGFBR3 | NM_003243.3 | Hs.482390 | 2.24 |
| 183 | Homo sapiens ADAMTS-like 4 | ADAMTSL4 | NM_019032.4 | Hs.516243 | 2.23 |
| 184 | Homo sapiens AF4/FMR2 family, member 3 | AFF3 | NM_002285.2 | Hs.444414 | 2.21 |
| 185 | Homo sapiens superkiller viralicidic activity 2-like (S. cerevisiae) | SKIV2L | NM_006929.4 | Hs.89864 | 2.18 |
| 186 | Homo sapiens CDC42 effector protein (Rho GTPase binding) 3 | CDC42EP3 | NM_006449.3 | Hs.369574 | 2.17 |
| 187 | Homo sapiens coiled-coil domain containing 8 | CCDC8 | NM_032040.3 | Hs.97876 | 2.13 |
| 188 | Homo sapiens kinesin family member 26A | KIF26A | NM_015656.1 | Hs.134970 | 2.09 |
| 189 | Homo sapiens hydroxysteroid (17-beta) dehydrogenase 12 | HSD17B12 | NM_016142.2 | Hs.132513 | 2.09 |
| 190 | Homo sapiens interleukin 7 | IL7 | NM_000880.2 | Hs.591873 | 2.09 |
| 191 | Homo sapiens prolyl-tRNA synthetase 2, mitochondrial (putative) | PARS2 | NM_152268.2 | Hs.380169 | 2.07 |

TABLE 7
Downexpressed candidates (29) of the hybridization on our custom-made library with their fold change

| | | Official | Genbank | Unigene | Fold |
|---|---|---|---|---|---|
| Order | Gene full name | symbol | accession | accession | Change |
| 1 | Homo sapiens ADP-ribosylation factor GTPase activating protein 3 | ARFGAP3 | NM_014570.4 | Hs.162877 | 3.145 |
| 2 | Homo sapiens transmembrane protein 219 | TMEM219 | NM_194280.3 | Hs.460574 | 2.991 |
| 3 | Homo sapiens archain 1 | ARCN1 | NM_001655.4 | Hs.33642 | 2.863 |
| 4 | Homo sapiens eukaryotic translation initiation factor 1 | EIF1 | BC008710 | Hs.150580 | 2.882 |
| 5 | Homo sapiens ribosomal protein L19 | RPL19 | BC066315 | Hs.381061 | 2.66 |
| 6 | Homo sapiens solute carrier family 25 (mitochondrial carrier; Phosphate | SLC25A3 | NM_213611 | Hs.290404 | 2.522 |
| | carrier), member 3 | | | | |
| 7 | Homo sapiens annexin A2 | ANXA2 | NM_001002858.2 | Hs.511605 | 2.458 |
| 8 | Homo sapiens ELK4, ETS-domain protein (SRF accessory protein 1) | ELK4 | NM_021795.2 | Hs.497520 | 2.39 |
| 9 | Homo sapiens interferon induced transmembrane protein 3 (1-8U) | IFITM3 | NM_021034.2 | Hs.374650 | 2.306 |
| 10 | Homo sapiens CCR4-NOT transcription complex, subunit 1 | CNOT1 | NM_016284 | Hs.716474 | 2.298 |
| 11 | Homo sapiens ubiquitin domain containing 2 | UBTD2 | NM_152277 | Hs.131570 | 2.296 |
| 12 | Homo sapiens FBJ murine osteosarcoma viral oncogene homolog B | FOSB | NM_001114171 | Hs.590958 | 2.192 |
| 13 | PREDICTED: Homo sapiens similar to OK/SW-CL.16 (LOC100288418) | LOC100288418 | XM_002342023.1 | GeneID: | 2.183 |
| | | | | 100288418 | |
| 14 | Homo sapiens phosphodiesterase 4D interacting protein | PDE4DIP | NM_022359.4 | Hs.719077 | 2.169 |
| 15 | PREDICTED: Homo sapiens similar to cytochrome c oxidase subunit II | LOC100293593 | XR_078889.1 | GeneID: | 2.162 |
| | | | | 100293593 | |
| 16 | Homo sapiens similar to cytochrome b (LOC100288871), miscRNA | LOC100288871 | XR_078322.1 | GeneID: | 2.122 |
| | | | | 100288871 | |
| 17 | Homo sapiens spermine synthase | SMS | NM_004595.2 | Hs.715555 | 2.104 |
| 18 | Human farnesyl pyrophosphate synthetase mRNA | FDPS | J05262 | Hs.335918 | 2.086 |
| 19 | Homo sapiens cytochrome P450, family 11, subfamily A, polypeptide | CYP11A1 | BC032329 | Hs.303980 | 2.079 |
| 20 | Homo sapiens methylmalonic aciduria (cobalamin deficiency) cblD type, with | MMADHC | NM_015702.2 | Hs.5324 | 2.004 |
| | homocystinuria | | | | |
| 21 | Homo sapiens complement component 9 | C9 | K02766 | Hs.654443 | 2.039 |
| 22 | Homo sapiens chromosome 20 open reading frame 4 | C20orf4 | NM_015511.3 | Hs.11314 | 2.031 |
| 23 | Homo sapiens ovarian tumor suppressor candidate 2 | OVCA2 | NM_080822.2 | Hs.513856 | 2.023 |
| 24 | PREDICTED: Homo sapiens similar to DC24 | LOC100293090 | XR_078993.1 | GeneID: | 2.016 |
| | | | | 100293090 | |
| 25 | Homo sapiens protein phosphatase 1A (formerly 2C), magnesium- | PPM1A | NM_177951.2 | Hs.130036 | 2 |
| | dependent, alpha isoform | | | | |

| No. | Unkown transcripts' sequences | length (pb) | Fold change |
|---|---|---|---|
| 1 | SEQ ID NO: 106 | 693 | 2 |
| 2 | SEQ ID NO: 107 | 275 | 2.3 |
| 3 | SEQ ID NO: 108 | 475 | 2.4 |
| 4 | SEQ ID NO: 109 | 273 | 3 |

TABLE 8
Downexpressed candidates (202) of the hybridization on the OneArray library with their fold change

| | | Official | Genbank | Unigene | Fold |
|---|---|---|---|---|---|
| No. | Gene full name | symbol | accession | accession | change |
| 1 | Homo sapiens golgi autoantigen, golgin subfamily a, 8B | GOLGA8B | NM_001023567.4 | Hs.182982 | 8.192 |
| 2 | Homo sapiens heat shock 60 kDa protein 1 (chaperonin) | HSPD1 | NM_199440.1 | Hs.719142 | 7.081 |
| 3 | Homo sapiens platelet derived growth factor C | PDGFC | NM_016205.1 | Hs.570855 | 6.392 |
| 4 | Homo sapiens stearoyl-CoA desaturase (delta-9-desaturase) | SCD | NM_005063.4 | Hs.558396 | 5.758 |
| 5 | Homo sapiens guanine nucleotide binding protein (G protein), gamma | GNG10 | NM_001017998.2 | Hs.534196 | 5.438 |
| | 10 | | | | |
| 6 | Homo sapiens SPHK1 interactor, AKAP domain containing | SPHKAP | NM_001142644.1 | Hs.436306 | 5.162 |
| 7 | Homo sapiens chromogranin B (secretogranin 1) | CHGB | NM_001819.2 | Hs.516874 | 5.151 |
| 8 | Homo sapiens growth factor receptor-bound protein 14 | GRB14 | NM_004490.2 | Hs.411881 | 5.093 |
| 9 | Homo sapiens RNA binding motif protein 39 | RBM39 | NM_184234.1 | Hs.282901 | 5.067 |
| 10 | Homo sapiens chromosome 4 open reading frame 3 | C4orf3 | NM_001001701.3 | Hs.718451 | 5.004 |
| 11 | Homo sapiens chromosome 1 open reading frame 63 | C1orf63 | NM_020317.3 | Hs.259412 | 4.967 |
| 12 | Homo sapiens baculoviral IAP repeat-containing 3 | BIRC3 | NM_001165.3 | Hs.127799 | 4.817 |
| 13 | Homo sapiens lysyl oxidase-like 2 | LOXL2 | NM_002318.2 | Hs.626637 | 4.790 |
| 14 | Homo sapiens metastasis associated lung adenocarcinoma transcript | MALAT1 | NR_002819.2 | Hs.642877 | 4.605 |
| | 1 (non-protein coding) | | | | |
| 15 | Homo sapiens chromosome 20 open reading frame 199 | C20orf199 | NR_003605.1 | Hs.356766 | 4.491 |
| 16 | Homo sapiens chromosome 20 open reading frame 30 | C20orf30 | NM_001009923.1 | Hs.719105 | 4.458 |
| 17 | Homo sapiens transmembrane 7 superfamily member 3 | TM7SF3 | NM_016551.2 | Hs.438641 | 4.413 |
| 18 | Homo sapiens proteasome (prosome, macropain) 26S subunit, | PSMC6 | NM_002806.3 | Hs.156171 | 4.374 |
| | ATPase, 6 | | | | |
| 19 | Homo sapiens ubiquitin-conjugating enzyme E2D 3 (UBC4/5 | UBE2D3 | NM_181893.1 | Hs.518773 | 4.245 |
| | homolog, yeast) | | | | |
| 20 | Homo sapiens ferredoxin 1 | FDX1 | NM_004109.3 | Hs.744 | 4.194 |
| 21 | Homo sapiens TGF beta-inducible nuclear protein 1 | TINP1 | NM_014886.3 | Hs.482526 | 4.138 |
| 22 | Homo sapiens glutathione S-transferase alpha 5 | GSTA5 | NM_153699.1 | Hs.646984 | 4.113 |
| 23 | Homo sapiens connective tissue growth factor | CTGF | NM_001901.2 | Hs.591346 | 4.096 |
| 24 | Homo sapiens chemokine (C-X-C motif) ligand 1 (melanoma growth | CXCL1 | NM_001511.1 | Hs.789 | 4.077 |
| | stimulating activity, alpha) | | | | |
| 25 | Homo sapiens RAB1A, member RAS oncogene family | RAB1A | NM_004161.4 | Hs.310645 | 4.047 |
| 26 | Homo sapiens cancer susceptibility candidate 4 | CASC4 | NM_138423.2 | Hs.512867 | 4.007 |
| 27 | Homo sapiens ERO1-like (S. cerevisiae) | ERO1L | NM_014584.1 | Hs.592304 | 4.007 |
| 28 | Homo sapiens Meis homeobox 2 | MEIS2 | NM_170676.2 | Hs.510989 | 3.957 |
| 29 | Homo sapiens MAK16 homolog (S. cerevisiae) | MAK16 | NM_032509.3 | Hs.583805 | 3.920 |
| 30 | Homo sapiens chromosome 19 open reading frame 2 | C19orf2 | NM_003796.2 | Hs.466391 | 3.917 |
| 31 | Homo sapiens kelch-like 4 (Drosophila) | KLHL4 | NM_019117.4 | Hs.49075 | 3.912 |
| 32 | Homo sapiens activating transcription factor 3 | ATF3 | NM_001030287.2 | Hs.460 | 3.862 |
| 33 | Homo sapiens fibrillin 1 | FBN1 | NM_000138.3 | Hs.591133 | 3.804 |
| 34 | Homo sapiens protein S (alpha) | PROS1 | NM_000313.3 | Hs.64016 | 3.799 |
| 35 | Homo sapiens major facilitator superfamily domain containing 1 | MFSD1 | NM_022736.1 | Hs.58663 | 3.703 |
| 36 | Homo sapiens cytochrome c oxidase subunit VIc | COX6C | NM_004374.2 | Hs.351875 | 3.699 |
| 37 | Homo sapiens metaxin 2 | MTX2 | NM_006554.4 | Hs.470728 | 3.676 |
| 38 | Homo sapiens DEAD (Asp-Glu-Ala-Asp) box polypeptide 5 | DDX5 | NM_004396.3 | Hs.279806 | 3.588 |
| 39 | Homo sapiens H3 histone, family 3A | H3F3A | NM_002107.3 | Hs.546259 | 3.570 |
| 40 | Homo sapiens KIAA0528 | KIAA0528 | NM_014802.1 | Hs.271014 | 3.553 |
| 41 | Homo sapiens coiled-coil domain containing 50 | CCDC50 | NM_178335.2 | Hs.478682 | 3.526 |
| 42 | Homo sapiens chaperonin containing TCP1, subunit 4 (delta) | CCT4 | NM_006430.2 | Hs.421509 | 3.503 |
| 43 | Homo sapiens collagen, type V, alpha 2 | COL5A2 | NM_000393.3 | Hs.445827 | 3.486 |
| 44 | Homo sapiens guanine nucleotide binding protein (G protein), beta | GNB4 | NM_021629.3 | Hs.173030 | 3.480 |
| | polypeptide 4 | | | | |
| 45 | Homo sapiens up-regulated during skeletal muscle growth 5 homolog | USMG5 | NM_032747.2 | Hs.500921 | 3.456 |
| | (mouse) | | | | |
| 46 | Homo sapiens hyaluronoglucosaminidase 4 | HYAL4 | NM_012269.2 | Hs.28673 | 3.448 |
| 47 | Homo sapiens nucleoporin 35 kDa | NUP35 | NM_138285.3 | Hs.180591 | 3.443 |
| 48 | Homo sapiens RAB23, member RAS oncogene family | RAB23 | NM_016277.3 | Hs.555016 | 3.382 |
| 49 | Homo sapiens ATP synthase, H+ transporting, mitochondrial F1 | ATP5E | NM_001001977.1 | Hs.177530 | 3.351 |
| | complex, epsilon subunit gene 2 | | | | |
| 50 | Homo sapiens solute carrier family 38, member 1 | SLC38A1 | NM_030674.3 | Hs.694701 | 3.347 |
| 51 | Homo sapiens HD domain containing 2 | HDDC2 | NM_016063.2 | Hs.32826 | 3.337 |
| 52 | Homo sapiens SMT3 suppressor of mif two 3 homolog 3 (S. | SUMO3 | NM_006936.2 | Hs.474005 | 3.331 |
| | cerevisiae) | | | | |
| 53 | Homo sapiens insulin-like growth factor binding protein 7 | IGFBP7 | NM_001553.1 | Hs.479808 | 3.315 |
| 54 | Homo sapiens N-glycanase 1 | NGLY1 | NM_001145294.1 | Hs.368960 | 3.262 |
| 55 | Homo sapiens guanine nucleotide binding protein (G protein), alpha | GNA13 | NM_006572.4 | Hs.515018 | 3.238 |
| | 13 | | | | |
| 56 | Homo sapiens USO1 homolog, vesicle docking protein (yeast) | USO1 | NM_003715.2 | Hs.292689 | 3.220 |
| 57 | Homo sapiens major histocompatibility complex, class I, B | HLA-B | NM_005514.6 | Hs.77961 | 3.219 |
| 58 | Homo sapiens nudix (nucleoside diphosphate linked moiety X)-type | NUDT9 | NM_024047.3 | Hs.149500 | 3.214 |
| | motif 9 | | | | |
| 59 | Homo sapiens carbonyl reductase 1 | CBR1 | NM_001757.2 | Hs.88778 | 3.214 |
| 60 | Homo sapiens haloacid dehalogenase-like hydrolase domain | HDHD2 | NM_032124.4 | Hs.465041 | 3.212 |
| | containing 2 | | | | |
| 61 | Homo sapiens syntaxin 3 | STX3 | NM_004177.3 | Hs.180711 | 3.187 |
| 62 | Homo sapiens echinoderm microtubule associated protein like 4 | EML4 | NM_019063.3 | Hs.593614 | 3.182 |
| 63 | Homo sapiens ornithine aminotransferase (gyrate atrophy) | OAT | NM_000274.2 | Hs.523332 | 3.146 |
| 64 | Homo sapiens nuclear undecaprenyl pyrophosphate synthase 1 | NUS1 | NM_138459.3 | Hs.289008 | 3.133 |
| | homolog (S. cerevisiae) | | | | |
| 65 | Homo sapiens thymine-DNA glycosylase | TDG | NM_003211.4 | Hs.584809 | 3.133 |
| 66 | Homo sapiens succinate dehydrogenase complex, subunit D, integral | SDHD | NM_003002.2 | Hs.719164 | 3.118 |
| | membrane protein | | | | |
| 67 | Homo sapiens hypothetical LOC400657, non-coding RNA | LOC400657 | NR_024484.1 | Hs.61508 | 3.112 |
| 68 | Homo sapiens NADH dehydrogenase (ubiquinone) 1 beta | NDUFB10 | NM_004548.2 | Hs.513266 | 3.078 |
| | subcomplex, 10, 22 kDa | | | | |
| 69 | Homo sapiens bromodomain and WD repeat domain containing 2 | BRWD2 | NM_018117.10 | Hs.144447 | 3.069 |
| 70 | Homo sapiens caveolin 1, caveolae protein, 22 kDa | CAV1 | NM_001753.3 | Hs.74034 | 3.038 |
| 71 | Homo sapiens secretogranin V (7B2 protein) | SCG5 | NM_001144757.1 | Hs.156540 | 3.037 |
| 72 | Homo sapiens progesterone receptor membrane component 1 | PGRMC1 | NM_006667.3 | Hs.90061 | 3.029 |
| 73 | Homo sapiens adenylosuccinate synthase | ADSS | NM_001126.2 | Hs.498313 | 3.018 |
| 74 | Homo sapiens nanos homolog 1 (Drosophila) | NANOS1 | NM_199461.2 | Hs.591918 | 3.017 |
| 75 | Homo sapiens ribosomal protein L17 | RPL17 | NM_000985.3 | Hs.374588 | 3.006 |
| 76 | Homo sapiens ADP-ribosylation-like factor 6 interacting protein 5 | ARL6IP5 | NM_006407.3 | Hs.716493 | 3.005 |
| 77 | Homo sapiens profilin 2 | PFN2 | NM_002628.4 | Hs.91747 | 2.938 |
| 78 | Homo sapiens prolyl endopeptidase-like | PREPL | NM_006036.3 | Hs.719111 | 2.908 |
| 79 | Homo sapiens ubiquitin-like with PHD and ring finger domains 2 | UHRF2 | NM_152896.1 | Hs.493401 | 2.902 |
| 80 | Homo sapiens chromodomain helicase DNA binding protein 6 | CHD6 | NM_032221.3 | Hs.371979 | 2.874 |
| 81 | Homo sapiens splicing factor, arginine/serine-rich 12 | SFRS12 | NM_001077199.1 | Hs.519347 | 2.868 |
| 82 | Homo sapiens translocation associated membrane protein 1 | TRAM1 | NM_014294.4 | Hs.491988 | 2.863 |
| 83 | Homo sapiens cytochrome c oxidase subunit VIIa polypeptide 2 (liver) | COX7A2 | NM_001865.2 | Hs.70312 | 2.857 |
| 84 | Homo sapiens RAD17 homolog (S. pombe) | RAD17 | NM_133343.1 | Hs.16184 | 2.850 |
| 85 | Homo sapiens paraoxonase 2 | PON2 | NM_000305.2 | Hs.719159 | 2.820 |
| 86 | Homo sapiens acid phosphatase, prostate | ACPP | NM_001099.4 | Hs.433060 | 2.819 |
| 87 | Homo sapiens zinc finger protein 330 | ZNF330 | NM_014487.4 | Hs.120766 | 2.802 |
| 88 | Homo sapiens RB1-inducible coiled-coil 1 | RB1CC1 | NM_014781.4 | Hs.196102 | 2.795 |
| 89 | Homo sapiens ELOVL family member 5, elongation of long chain fatty | ELOVL5 | NM_021814.3 | Hs.713560 | 2.795 |
| | acids (FEN1/Elo2, SUR4/Elo3-like, yeast) | | | | |
| 90 | Homo sapiens gap junction protein, alpha 1, 43 kDa | GJA1 | NM_000165.3 | Hs.74471 | 2.773 |
| 91 | Homo sapiens density-regulated protein | DENR | NM_003677.3 | Hs.22393 | 2.748 |
| 92 | Homo sapiens KH domain containing, RNA binding, signal | KHDRBS1 | NM_006559.1 | Hs.709204 | 2.745 |
| | transduction associated 1 | | | | |
| 93 | Homo sapiens stearoyl-CoA desaturase 5 | SCD5 | NM_001037582.2 | Hs.379191 | 2.743 |
| 94 | Homo sapiens antizyme inhibitor 1 | AZIN1 | NM_015878.4 | Hs.459106 | 2.741 |
| 95 | Homo sapiens integrin beta 1 binding protein 1 | ITGB1BP1 | NM_004763.3 | Hs.467662 | 2.734 |
| 96 | Homo sapiens leprecan-like 1 | LEPREL1 | NM_018192.3 | Hs.374191 | 2.725 |
| 97 | Homo sapiens nuclear factor (erythroid-derived 2)-like 2 | NFE2L2 | NM_006164.3 | Hs.715540 | 2.722 |
| 98 | Homo sapiens DnaJ (Hsp40) homolog, subfamily C, member 1 | DNAJC1 | NM_022365.3 | Hs.499000 | 2.719 |
| 99 | Homo sapiens tropomyosin 4 | TPM4 | NM_001145160.1 | Hs.631618 | 2.717 |
| 100 | Homo sapiens suppression of tumorigenicity 13 (colon carcinoma) | ST13 | NM_003932.3 | Hs.712713 | 2.712 |
| | (Hsp70 interacting protein) | | | | |
| 101 | Homo sapiens vascular endothelial growth factor C | VEGFC | NM_005429.2 | Hs.435215 | 2.671 |
| 102 | Homo sapiens protein O-fucosyltransferase 2 | POFUT2 | NM_133635.4 | Hs.592164 | 2.635 |
| 103 | Homo sapiens proline-rich nuclear receptor coactivator 2 | PNRC2 | NM_017761.3 | Hs.512636 | 2.632 |
| 104 | Homo sapiens ubiquitin-conjugating enzyme E2B (RAD6 homolog) | UBE2B | NM_003337.2 | Hs.644421 | 2.621 |
| 105 | Homo sapiens DDB1 and CUL4 associated factor 12 | DCAF12 | NM_015397.3 | Hs.716472 | 2.617 |
| 106 | Homo sapiens DnaJ (Hsp40) homolog, subfamily C, member 24 | DNAJC24 | NM_181706.4 | Hs.718544 | 2.579 |
| 107 | Homo sapiens perilipin 2 | PLIN2 | NM_001122.2 | Hs.3416 | 2.570 |
| 108 | Homo sapiens thioredoxin | TXN | NM_003329.2 | Hs.435136 | 2.569 |
| 109 | Homo sapiens amylo-1, 6-glucosidase, 4-alpha-glucanotransferase | AGL | NM_000642.2 | Hs.904 | 2.562 |
| 110 | Homo sapiens N-acetylglucosamine-1-phosphate transferase, | GNPTG | NM_032520.3 | Hs.241575 | 2.559 |
| | gamma subunit | | | | |
| 111 | Homo sapiens tyrosylprotein sulfotransferase 1 | TPST1 | NM_003596.3 | Hs.421194 | 2.555 |
| 112 | Homo sapiens cytochrome c, somatic | CYCS | NM_018947.4 | Hs.437060 | 2.544 |
| 113 | Homo sapiens palladin, cytoskeletal associated protein | PALLD | NM_016081.3 | Hs.151220 | 2.520 |
| 114 | Homo sapiens transmembrane protein 14A | TMEM14A | NM_014051.3 | Hs.94896 | 2.509 |
| 115 | Homo sapiens ADP-ribosylation factor GTPase activating protein 3 | ARFGAP3 | NM_014570.4 | Hs.162877 | 2.505 |
| 116 | Homo sapiens SECIS binding protein 2-like | SECISBP2L | NM_014701.2 | Hs.9997 | 2.504 |
| 117 | Homo sapiens mitogen-activated protein kinase 6 | MAPK6 | NM_002748.3 | Hs.411847 | 2.503 |
| 118 | Homo sapiens RNA binding motif protein, X-linked | RBMX | NM_002139.2 | Hs.380118 | 2.501 |
| 119 | Homo sapiens CD164 molecule, sialomucin | CD164 | NM_001142403.1 | Hs.520313 | 2.499 |
| 120 | Homo sapiens pyrophosphatase (inorganic) 1 | PPA1 | NM_021129.3 | Hs.437403 | 2.493 |
| 121 | Homo sapiens DIRAS family, GTP-binding RAS-like 3 | DIRAS3 | NM_004675.2 | Hs.194695 | 2.483 |
| 122 | Homo sapiens glutathione S-transferase omega 1 | GSTO1 | NM_004832.1 | Hs.190028 | 2.480 |
| 123 | Homo sapiens arginase, type II | ARG2 | NM_001172.3 | Hs.708024 | 2.478 |
| 124 | Homo sapiens headcase homolog (Drosophila) | HECA | NM_016217.2 | Hs.197644 | 2.477 |
| 125 | Homo sapiens heat shock 70 kDa protein 12A | HSPA12A | NM_025015.2 | Hs.654682 | 2.468 |
| 126 | Homo sapiens mitochondrial intermediate peptidase | MIPEP | NM_005932.2 | Hs.507498 | 2.463 |
| 127 | Homo sapiens defender against cell death 1 | DAD1 | NM_001344.2 | Hs.82890 | 2.455 |
| 128 | omo sapiens microsomal glutathione S-transferase 1 | MGST1 | NM_145792.1 | Hs.389700 | 2.454 |
| 129 | Homo sapiens SH3 domain containing 19 | SH3D19 | NM_001009555.3 | Hs.567725 | 2.447 |
| 130 | Homo sapiens myosin ID | MYO1D | NM_015194.1 | Hs.658000 | 2.440 |
| 131 | Homo sapiens COP9 constitutive photomorphogenic homolog subunit | COPS5 | NM_006837.2 | Hs.491912 | 2.427 |
| | 5 (Arabidopsis) | | | | |
| 132 | Homo sapiens programmed cell death 6 interacting protein | PDCD6IP | NM_013374.4 | Hs.475896 | 2.426 |
| 133 | Homo sapiens BTAF1 RNA polymerase II, B-TFIID transcription | BTAF1 | NM_003972.2 | Hs.500526 | 2.420 |
| | factor-associated, 170 kDa (Mot1 homolog, S. cerevisiae) | | | | |
| 134 | Homo sapiens ArfGAP with SH3 domain, ankyrin repeat and PH | ASAP2 | NM_003887.2 | Hs.555902 | 2.419 |
| | domain 2 | | | | |
| 135 | Homo sapiens cytidine monophosphate (UMP-CMP) kinase 1, | CMPK1 | NM_016308.2 | Hs.714325 | 2.418 |
| | cytosolic | | | | |
| 136 | Homo sapiens KN motif and ankyrin repeat domains 1 | KANK1 | NM_015158.2 | Hs.306764 | 2.409 |
| 137 | Homo sapiens Sjogren syndrome antigen B (autoantigen La) | SSB | NM_003142.3 | Hs.632535 | 2.403 |
| 138 | Homo sapiens Alstrom syndrome 1 | ALMS1 | NM_015120.4 | Hs.184720 | 2.402 |
| 139 | Homo sapiens peroxiredoxin 4 | PRDX4 | NM_006406.1 | Hs.83383 | 2.401 |
| 140 | Homo sapiens iron-sulfur cluster scaffold homolog (E. coli) | ISCU | NM_213595.2 | Hs.615131 | 2.392 |
| 141 | Homo sapiens caspase 9, apoptosis-related cysteine peptidase | CASP9 | NM_001229.2 | Hs.329502 | 2.380 |
| 142 | Homo sapiens Werner helicase interacting protein 1 | WRNIP1 | NM_020135.2 | Hs.236828 | 2.375 |
| 143 | Homo sapiens carnitine palmitoyltransferase 2 | CPT2 | NM_000098.2 | Hs.713535 | 2.365 |
| 144 | Homo sapiens splicing factor, arginine/serine-rich 7, 35 kDa | SFRS7 | NM_001031684.2 | Hs.309090 | 2.364 |
| 145 | Homo sapiens Rab geranylgeranyltransferase, beta subunit | RABGGTB | NM_004582.2 | Hs.78948 | 2.356 |
| 146 | Homo sapiens PHD finger protein 3 | PHF3 | NM_015153.2 | Hs.348921 | 2.347 |
| 147 | Homo sapiens phosphatidylinositol glycan anchor biosynthesis, class G | PIGG | NM_017733.3 | Hs.7099 | 2.345 |
| 148 | Homo sapiens topoisomerase (DNA) II binding protein 1 | TOPBP1 | NM_007027.3 | Hs.53454 | 2.340 |
| 149 | Homo sapiens ELOVL family member 6, elongation of long chain fatty | ELOVL6 | NM_001130721.1 | Hs.412939 | 2.339 |
| | acids (FEN1/Elo2, SUR4/Elo3-like, yeast) | | | | |
| 150 | Homo sapiens glyceronephosphate O-acyltransferase | GNPAT | NM_014236.3 | Hs.498028 | 2.337 |
| 151 | Homo sapiens SEC24 family, member B (S. cerevisiae) | SEC24B | NM_006323.2 | Hs.292472 | 2.332 |
| 152 | Homo sapiens A kinase (PRKA) anchor protein (yotiao) 9 | AKAP9 | NM_005751.4 | Hs.651221 | 2.330 |
| 153 | Homo sapiens ATG3 autophagy related 3 homolog (S. cerevisiae) | ATG3 | NM_022488.3 | Hs.477126 | 2.329 |
| 154 | Homo sapiens golgi autoantigen, golgin subfamily a, 1 | GOLGA1 | NM_002077.3 | Hs.133469 | 2.328 |
| 155 | Homo sapiens ubiquitin specific peptidase 14 (tRNA-guanine | USP14 | NM_005151.3 | Hs.464416 | 2.323 |
| | transglycosylase) | | | | |
| 156 | Homo sapiens myeloid/lymphoid or mixed-lineage leukemia (trithorax | MLLT11 | NM_006818.3 | Hs.75823 | 2.322 |
| | homolog, Drosophila); translocated to, 11 | | | | |
| 157 | Homo sapiens pentraxin-related gene, rapidly induced by IL-1 beta | PTX3 | NM_002852.3 | Hs.591286 | 2.319 |
| 158 | Homo sapiens endoplasmic reticulum lectin 1 | ERLEC1 | NM_015701.3 | Hs.438336 | 2.314 |
| 159 | Homo sapiens FK506 binding protein 3, 25 kDa | FKBP3 | NM_002013.3 | Hs.509226 | 2.309 |
| 160 | Homo sapiens acidic repeat containing | ACRC | NM_052957.4 | Hs.135167 | 2.294 |
| 161 | Homo sapiens cathepsin A | CTSA | NM_000308.2 | Hs.609336 | 2.293 |
| 162 | Homo sapiens chromosome 1 open reading frame 151 | C1orf151 | NM_001032363.1 | Hs.466662 | 2.278 |
| 163 | Homo sapiens sec1 family domain containing 1 | SCFD1 | NM_016106.2 | Hs.369168 | 2.276 |
| 164 | Homo sapiens major histocompatibility complex, class II, DQ beta 2 | HLA-DQB2 | NR_003937.1 | Hs.409934 | 2.275 |
| 165 | Homo sapiens phosphoserine phosphatase | PSPH | NM_004577.3 | Hs.512656 | 2.266 |
| 166 | Homo sapiens malic enzyme 1, NADP(+)-dependent, cytosolic | ME1 | NM_002395.3 | Hs.21160 | 2.263 |
| 167 | Homo sapiens regulation of nuclear pre-mRNA domain containing 1A | RPRD1A | NM_018170.3 | Hs.464912 | 2.243 |
| 168 | Homo sapiens membrane-associated ring finger (C3HC4) 7 | MARCH7 | NM_022826.2 | Hs.529272 | 2.237 |
| 169 | Homo sapiens ribosomal protein L4 | RPL4 | NM_000968.2 | Hs.644628 | 2.228 |
| 170 | Homo sapiens TATA box binding protein (TBP)-associated factor, | TAF1C | NM_005679.2 | Hs.153022 | 2.222 |
| | RNA polymerase I, C, 110 kDa | | | | |
| 171 | Homo sapiens phosphatidylinositol glycan anchor biosynthesis, class Y | PIGY | NM_032906.3 | Hs.26136 | 2.222 |
| 172 | Homo sapiens mastermind-like domain containing 1 | MAMLD1 | NM_005491.2 | Hs.20136 | 2.220 |
| 173 | Homo sapiens Down syndrome critical region gene 3 | DSCR3 | NM_006052.1 | Hs.369488 | 2.212 |
| 174 | Homo sapiens sorting nexin 2 | SNX2 | NM_003100.2 | Hs.713554 | 2.212 |
| 175 | Homo sapiens chromosome 5 open reading frame 23 | C5orf23 | NM_024563.3 | Hs.13528 | 2.209 |
| 176 | Homo sapiens adenosine kinase | ADK | NM_006721.2 | Hs.656586 | 2.208 |
| 177 | Homo sapiens inhibitor of Bruton agammaglobulinemia tyrosine | IBTK | NM_015525.2 | Hs.306425 | 2.205 |
| | kinase | | | | |
| 178 | Homo sapiens claudin 1 | CLDN1 | NM_021101.3 | Hs.439060 | 2.204 |
| 179 | Homo sapiens sterile alpha motif and leucine zipper containing kinase | ZAK | NM_133646.2 | Hs.444451 | 2.202 |
| | AZK | | | | |
| 180 | Homo sapiens brix domain containing 5 | BXDC5 | NM_025065.6 | Hs.481202 | 2.201 |
| 181 | Homo sapiens Rho GTPase activating protein 6 | ARHGAP6 | NM_013427.2 | Hs.435291 | 2.201 |
| 182 | Homo sapiens tripeptidyl peptidase I | TPP1 | NM_000391.3 | Hs.523454 | 2.200 |
| 183 | Homo sapiens ribosomal protein L26-like 1 | RPL26L1 | NM_016093.2 | Hs.546390 | 2.187 |
| 184 | Homo sapiens coiled-coil domain containing 53 | CCDC53 | NM_016053.2 | Hs.405692 | 2.175 |
| 185 | Homo sapiens palmitoyl-protein thioesterase 1 | PPT1 | NM_000310.3 | Hs.3873 | 2.173 |
| 186 | Homo sapiens neuroblastoma breakpoint family, member 3 | NBPF3 | NM_032264.2 | Hs.325422 | 2.164 |
| 187 | Homo sapiens glutamine-fructose-6-phosphate transaminase 1 | GFPT1 | NM_002056.2 | Hs.580300 | 2.157 |
| 188 | Homo sapiens SGT1, suppressor of G2 allele of SKP1 (S. cerevisiae) | SUGT1 | NM_001130912.1 | Hs.281902 | 2.152 |
| 189 | Homo sapiens protein tyrosine phosphatase-like (proline instead of | PTPLA | NM_014241.3 | Hs.114062 | 2.137 |
| | catalytic arginine), member A | | | | |
| 190 | Homo sapiens hypoxanthine phosphoribosyltransferase 1 | HPRT1 | NM_000194.2 | Hs.412707 | 2.119 |
| 191 | Homo sapiens golgi autoantigen, golgin subfamily a, 5 | GOLGA5 | NM_005113.2 | Hs.104320 | 2.117 |
| 192 | Homo sapiens sterol-C5-desaturase (ERG3 delta-5-desaturase | SC5DL | NM_006918.4 | Hs.287749 | 2.117 |
| | homolog, S. cerevisiae)-like | | | | |
| 193 | Homo sapiens interleukin 8 | IL8 | NM_000584.2 | Hs.624 | 2.114 |
| 194 | Homo sapiens small nuclear ribonucleoprotein 27 kDa (U4/U6.U5) | SNRNP27 | NM_006857.1 | Hs.54649 | 2.105 |
| 195 | Homo sapiens MAPK scaffold protein 1 | MAPKSP1 | NM_021970.3 | Hs.716375 | 2.101 |
| 196 | Homo sapiens sphingomyelin synthase 1 | SGMS1 | NM_147156.3 | Hs.654698 | 2.098 |
| 197 | Homo sapiens cytidine monophosphate N-acetylneuraminic acid | CMAS | NM_018686.3 | Hs.311346 | 2.097 |
| | synthetase | | | | |
| 198 | Homo sapiens protein phosphatase 2 (formerly 2A), catalytic subunit, | PPP2CA | NM_002715.2 | Hs.105818 | 2.080 |
| | alpha isoform | | | | |
| 199 | Homo sapiens reticulocalbin 1, EF-hand calcium binding domain | RCN1 | NM_002901.2 | Hs.97887 | 2.075 |
| 200 | Homo sapiens ribosomal protein L7 | RPL7 | NM_000971.3 | Hs.571841 | 2.068 |
| 201 | Homo sapiens NADH dehydrogenase (ubiquinone) 1 alpha | NDUFA6 | NM_002490.3 | Hs.274416 | 2.058 |
| | subcomplex, 6, 14 kDa | | | | |
| 202 | Homo sapiens target of myb1 (chicken) | TOM1 | NM_001135732 | Hs.474705 | 1.58 |

### EXAMPLE 3

### Markers in Human Follicular Fluid Associated with Competent Oocytes

This example describes the purification of protein markers from the follicular fluid samples obtained from the same patients part of the study described in Example 1.

### Materials and Methods

### Depletion of Major Abundant Proteins and Sample Preparation

Protein concentrations in samples of follicular fluid were determined using BCA Protein Assay™ kit (Thermo Scientific, Rockford, Ill., USA). Depletion of twelve most abundant proteins (albumin, IgG, transferin, fibrinogen, IgA, α2-macroglobulin, IgM, α1-antitrypsin, haptoglobin, α1-acidic glycoprotein and apolipoproteins A-I a A-II) in follicular fluid was carried out using multiple affinity ProteomeLab™ IgY-12 LC10™ column (Beckman Coulter, Fullerton, Calif., USA) following manufacturer's instructions. The efficacy of high capacity IgY-12 LC-1O™ column was high removing 95-98% of original protein amount. One cycle provided in average 630 μg of proteins of follicular fluid. In total we performed six depletion cycles, three for each pool of samples (A and B). The proteins in flow-through fractions were precipitated by addition of 0.15% sodium deoxycholate for 10 minutes and 72% trichloroacetic acid for 30 minutes (both in 1/10 of total volume). After washing with ice-cold acetone, pellets were resolubilised in sample buffer containing 9 M urea, 3% w/v CHAPS, 2% v/v Nonidet 40, 70 mM DTT, pH 3-10 ampholytes (0.5% w/v), 10 mM beta-glycerol phosphate, 5 mM sodium fluoride, 0.1 mM sodium orthovanadate, and protease inhibitors.

### Two Dimensional Electrophoresis and Image Analysis

Aliquotes of samples of depleted follicular fluid corresponding to 180 μg of proteins were loaded on the first dimmension isoelectric focusing separation using active in gel rehydration of Immobiline DryStrips™ (IPG strip 18 cm 4-7) in rehydration buffer containing 5M urea, 2M thiourea, 2% CHAPS, 2 mM TCEP, 40 mM Tris-base, 0.003% bromophenol blue. After IEF separation the gel strips were equilibrated and applied to vertical 12% T acrylamide SDS-PAGE (18×18×1 mm gel). SDS-PAGE was carried out at a constant current of 40 mA per gel using two in series connected Protean II xi Cells™ (Bio-Rad, Hercules, Calif., USA) allowing simultaneous run of four gels. Gels were then stained with mass spectrometry compatible silver staining SilverQuest™ kit. Stained gels were scanned and digitized at 400 dpi resolution using a GS800™ scanner (Bio-Rad, Hercules, Calif., USA).

The images were evaluated using ImageMaster Platinum 6.0™ (GE Healthcare, Upsala, Sweden). Data were normalized, i.e. expressed as percentages of all valid spots, to account for any differences in protein loading and gel staining. Normalised data were analyzed using statistical procedures available within the software (T-test). The protein spots that were statistically significant with P<0.05 according to Student's t-tests were selected for identification by mass spectrometry.

### Enzymatic In-Gel Digestion

CBB- or silver nitrate-stained protein spots were excised from the gel, cut into small pieces and washed with 50 mM 4-ethylmorpholine acetate (pH 8.1) in 50% acetonitrile (MeCN). After complete destaining, the gel was washed with water, shrunk by dehydration in MeCN and reswelled again in water. The supernatant was removed and the gel was partly dried in a SpeedVac™ concentrator. The gel pieces were then rehydrated in a cleavage buffer containing 25 mM 4-ethylmorpholine acetate, 5% MeCN and trypsin (5 ng/μl; Promega, Madison, Wis.), and incubated overnight at 37° C. The digestion was stopped by addition of 5% trifluoroacetic acid (TFA) in MeCN and the aliquot of the resulting peptide mixture was desalted using a GELoader™ microcolumn (Eppendorf, Hamburg, Germany) packed with a Poros Oligo R3™ material [Gobom, J., Nordhoff, E., Mirgorodskaya, E., Ekman, R., and Roepstorff, P. (1999) Sample purification and preparation technique based on nanoscale reversed-phase columns for the sensitive analysis of complex peptide mixtures by matrix-assisted laser desorption/ionization mass spectrometry. J. Mass Spectrom. 34, 105-116]. The purified and concentrated peptides were eluted from the microcolumn in several droplets directly onto MALDI plate using 1 μl of α-cyano-4-hydroxycinnamic acid (CCA) matrix solution (5 mg/ml in 50% MeCN/0.1% TFA).

### MALDI Mass Spectrometry

Mass spectra were measured on an Ultraflex III™ MALDI-TOF/TOF instrument (Bruker Daltonics, Bremen, Germany) equipped with a Smartbeam™ solid state laser and LIFT™ technology for MS/MS analysis. PMF spectra were acquired in the mass range of 700-4000 Da and calibrated internally using the monoisotopic [M+H]⁺ ions of trypsin autoproteolytic fragments (842.5 and 2211.1 Da).

### Protein Identification

For PMF database searching, peak lists in XML data format were created using flexAnalysis 3.0™ program with SNAP peak detection algorithm. No smoothing was applied and maximal number of assigned peaks was set to 50. After peak labeling all known contaminant signals were removed. The peak lists were searched using in-house MASCOT™ search engine against SwissProt™ 57.0 database subset of human proteins with the following search settings: peptide tolerance of 30 ppm, missed cleavage site value set to two, variable carbamidomethylation of cysteine, oxidation of methionine and protein N-term acetylation. No restriction on protein molecular weight and pl value were applied. Proteins with MOWSE score over the threshold 56 calculated for the used settings were considered as identified. If the score was lower or only slightly higher than the threshold value, the identity of protein candidate was confirmed by MS/MS analysis. In addition to the above mentioned MASCOT™ settings fragment mass tolerance of 0.6 Da and instrument type MALDI-TOF-TOF was applied for MS/MS spectra searching.

### Immunoblot and Quantitative Analysis

Portions of the total protein extracts of follicular fluid (15 μg) are separated in SDS-PAGE gels using Protean II xi Cell™ (Bio-Rad, Hercules, Calif., USA). Proteins are then transferred to Immobilon P™ (Millipore, Bedford, Mass., USA) membranes using a semidry blotting system (Biometra, GOttingen, Germany) and transfer buffer containing 48 mM Tris, 39 mM glycine and 20% methanol. The membranes are blocked for 1 h with 3% skimmed milk in Tris-buffered saline with 0.05% Tween 20™ (TBST, pH 7.4) and incubated overnight with primary antibodies raised against APO A4 (Sigma Prestige Antibodies, St Louis, Mo., USA; HPA001352; 1:7500-10000) and Ceruloplasmin (Abcam Inc., Cambridge, UK, ab 51083; 1: 10000-2000). Peroxidase-conjugated secondary anti-mouse or anti-rabbit IgG antibodies (Jackson Immunoresearch, Suffolk, UK), as appropriate, are diluted 1:10000 in 3% skimmed milk in TBST, and the ECL+™ chemiluminiscence (GE Healthcare, Upsala, Sweden) detection system is used to detect specific proteins. The exposed CL-XPosure™ films (Thermo Scientific, Rockford, Ill., USA) are scanned by a calibrated densitometer GS-800™ (Bio-Rad, Hercules, Calif., USA). The proteins bands of each sample are quantified as Trace Quantity (the quantity of a band as measured by the area under its intensity profile curve, units are intensity×mm) using Quantity One™ software (Bio-Rad, Hercules, Calif., USA). Further immunoanalysis of APO A4 and ceruloplasmin isoforms is carried out by separating non-depleted lysates of follicular fluid samples containing 100-150 μg of protein, in 2-DE gels as described above. Narrow Immobiline DryStrips™ pH 4.7-5.9 7 cm (GE Healthcare, Upsala, Sweden) are used to analyse microheterogeneity of APO A4 and ceruloplasmin. Transfer of the proteins to membranes and immunodetection is performed as described above. Protein quantification using ImageMaster Platimun 6.0™ (GE Healthcare, Upsala, Sweden) is applied and 2DE data are expressed as relative spot volume of all spots representing particular protein.

### Results

Briefly, 3 pools A and 3 pools B were depleted individually using immunoaffinity IgY12 system (Beckman Coulter) removing 12 of the most abundant proteins that represented about 95% of original protein. The eluate (5% l-flow through) was separated by 2DE (18 cm IPG pH 4-7 and 20 cm SDS PAGE 12%) and stained by fluorescence stain (Sypro).

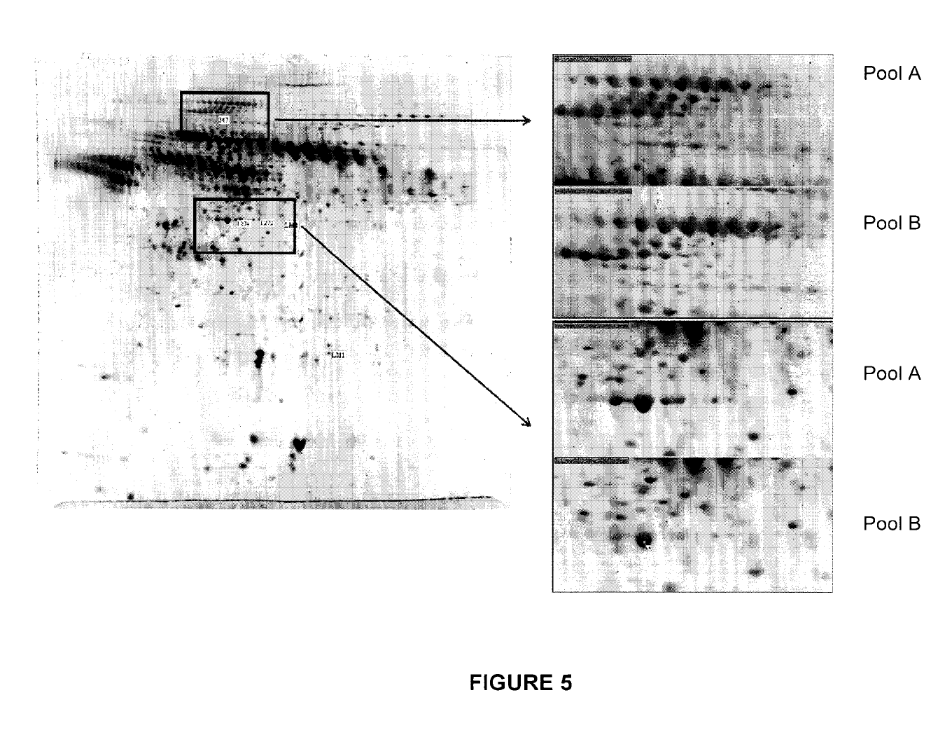

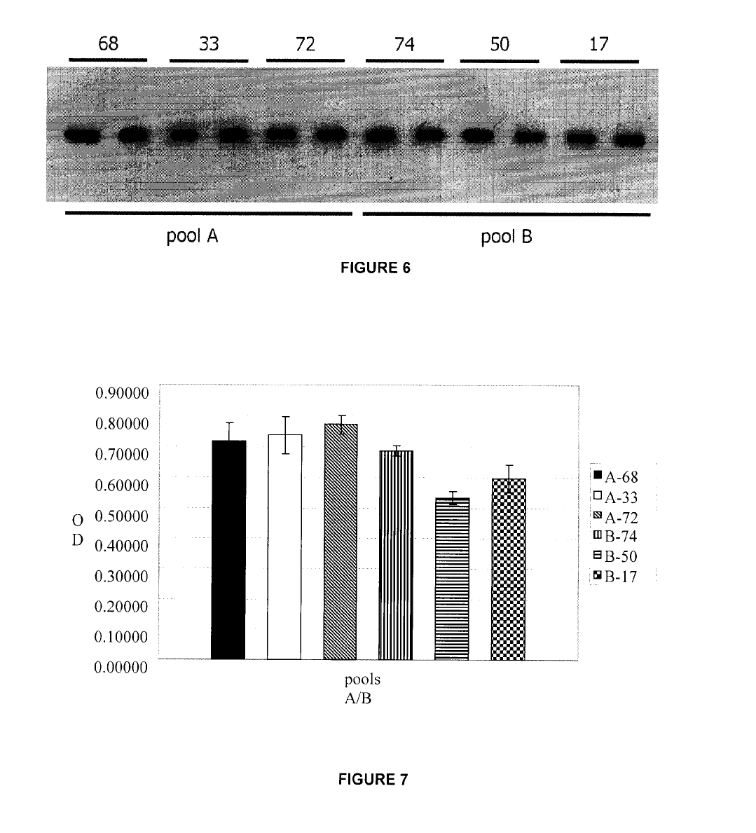

Each sample pool was run in 2 replicates, in total we had 12 gels—6A and 6B. The images were evaluated using Image Master™ software (GE Healthcare). The gels were of good quality based on spot resolution, numbers and matching, as well as scatter plots, unfortunately, it was very difficult to find differentially expressed spots—significant and reproducible. To be sure about images evaluation, two PhD students well experienced with software did the computer evaluation independently. Additionally, we send the images to Ludesi (www.ludesi.com) that is known to performing 2DE gel image analyses. This effort resulted in selection of three differentially expressed proteins (Tables 9 and 10, FIGS. 5 to 7) which have been analysed by MS (Table 4). Spot 347 appears to be upregulated in pool B, spot 1247 (acidic (Left) spot from two closely located spots in A pool) is upregulated in A pool and this “double” was never observed in B pool. Therefore, these two spots could actually be the same protein and be the consequence of post translational modifications. Spot 1272 is upregulated in A pool.

Altogether, this result indicate that the biological variances that are usually relatively high in individual samples are nearly eliminated to zero and the changes that we were able to find (not many) might represent apparently “typical” difference between follicular fluid from competent follicles or follicles with failure in development.

TABLE 9
List of proteins identified by the Mass spec analysis

| Spot | | SwissProt | No | Coverage | | | |
|---|---|---|---|---|---|---|---|
| No | Protein Name | No. | Peptides | (%) | LIFT | MW | pl |
| 347 | Ceruloplasmin | CERU_HUMAN | 4 | 5 | GAYPLSIEPIGVR ALYLQYTDETFR | 125 | 5.4 |
| | precursor | | | | (SEQ ID NO: 110) | | |
| 1272 | Actin, | ACTB_HUMAN | 4 | 12 | SYELPDGQVITIGNERQEYDESGPSIVHR | 42 | 5.3 |
| | cytoplasmic 1 | | | | (SEQ ID NO: 111) | | |
| | (β-actin or | | | | | | |
| | ACTB) | | | | | | |
| 1274A | Apolipoprotein | APOA4_HUMAN | 10 | 21 | LEPYADQLR | 47 | 5.3 |
| | A-IV precursor | | | | (SEQ ID NO: 112) | | |
| 1274B | Apolipoprotein | APOA4_HUMAN | 10 | 21 | No | 47 | 5.3 |
| | A-IV precursor | | | | | | |
| 1274C | Apolipoprotein | APOA4_HUMAN | 10 | 21 | No | 47 | 5.3 |
| | A-IV precursor | | | | | | |

TABLE 10
Sequence information of the proteins identified by the Mass spec analysis

| Spot No | Protein Name | SwissProt No. | Unigene | Genbank prot | Genbank nucl |
|---|---|---|---|---|---|
| 347 | Ceruloplasmin precursor | CERU_HUMAN | Hs.558314 | NP_000087.1 | NM_000096.3 |
| 1272 | Actin, cytoplasmic 1 | ACTB_HUMAN | Hs.520640 | NP_001092.1 | NM_001101.3 |
| | (β-actin or ACTB) | | | | |
| 1274A | Apolipoprotein A-IV precursor | APOA4_HUMAN | Hs.591940 | NP_000473.2 | NM_000482.3 |
| 1274B | Apolipoprotein A-IV precursor | APOA4_HUMAN | Hs.591940 | NP_000473.2 | NM_000482.3 |
| 1274C | Apolipoprotein A-IV precursor | APOA4_HUMAN | Hs.591940 | NP_000473.2 | NM_000482.3 |

### EXAMPLE 4

### Use of a Chip Comprising Antibodies for Evaluating Competence of a Mammalian Oocyte

This hypothetical example describes the use of a solid support such as a chip for evaluating the competence of a mammalian oocyte.

A chip (e.g. Ciphergen ProteinChip™) for measuring two or more predetermined ovarian markers is prepared using known methods (e.g. Lin et al., Application of SELDI-TOF mass spectrometry for the identification of differentially expressed proteins in transformed follicular lymphoma. Mod Pathol. 2004 June; 17(6):670-8; Wang et al., Mass spectrometric analysis of protein markers for ovarian cancer. Clin Chem. 2004 October; 50(10):1939-42; Simonsen et al., Amyloid beta 1-40 quantification in CSF: comparison between chromatographic and immunochemical methods. Dement Geriatr Cogn Disord. 2007; 23(4):246-50)

The chip comprises a plurality of antibodies types, each type being capable of specifically binding to a predetermined ovarian marker (e.g. specific for polypeptides expressed by the gene of interest). The chip is contacted with a cell lysate or with biological fluids from cumulus cells, biological fluids from follicular cells or follicular fluid. After a certain period the chip is rinsed for removing unbound non-specific material and it is submitted to mass spectrometry for quantification of the materials remaining on the chip. Results form the quantification measurements are inputted into a computer for analysis using a multivariable algorithm for obtaining a score. The score gives an indication of the competence of the mammalian oocyte.

### EXAMPLE 5

### Use of a DNA Chip for Evaluating Competence of a Mammalian Oocyte

This hypothetical example describes the use of a solid support such as a DNA chip for evaluating the competence of a mammalian oocyte.

A DNA chip (e.g. micro-array with cDNA or oligomers) for measuring two or more predetermined ovarian markers is prepared using known methods (e.g. Harry et al., Predicting the response of advanced cervical and ovarian tumors to therapy. Obstet Gynecol Surv. 2009 August; 64(8):548-60; Ross J S. Multigene classifiers, prognostic factors, and predictors of breast cancer clinical outcome. Adv Anat Pathol. 2009 July; 16(4):204-15; Sotiriou C and Pusztai L. Gene-expression signatures in breast cancer. N Engl J Med. 2009 Feb. 19; 360(8):790-800).

The chip comprises a plurality of specific DNA targets (each target being capable of specifically binding to a predetermined ovarian marker (e.g. a cDNA molecule or a aRNA molecule hybridizing specifically with a mRNA expressed by the gene of interest). The chip is contacted with a set of DNA targets (e.g. cDNA or mRNA molecules having about 20, 30, 40, 50, 60, 70 or more nucleotides) and probed with complementary DNA obtained by reverse transcription/amplification of the RNA expressed in the selected tissues (follicular or cumulus cells) to examine fluorescent dyes intensity. After a certain period the chip is rinsed for removing unbound non-specific material and it is submitted to laser in a slide reader for pixel quantification of the materials remaining on the chip. Results from the quantification measurements are inputted into a computer for analysis using a multivariable algorithm for obtaining a score. The score gives an indication of the competence of the mammalian oocyte.

Headings are included herein for reference and to aid in locating certain sections These headings are not intended to limit the scope of the concepts described therein under, and these concepts may have applicability in other sections throughout the entire specification Thus, the present invention is not intended to be limited to the embodiments shown herein but is to be accorded the widest scope consistent with the principles and novel features disclosed herein.

Unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contain certain errors resulting from variations in experiments, testing measurements, statistical analyses and such.

It is understood that the examples and embodiments described herein are for illustrative purposes only and that various modifications or changes in light thereof will be suggested to persons skilled in the art and are to be included within the present invention and scope of the appended claims.

1. (canceled)

2. (canceled)

3. (canceled)

4. (canceled)

5. A method for evaluating competence of a human oocyte, said method comprising assessing expression of at least one follicular cell marker which is expressed in follicular cells of an ovarian follicle comprising said mammalian oocyte, wherein said follicular cell marker is selected from the group consisting of UGP2, PHLDA1, GAPBP1, SFRP1, HOMER1, LRP8, DPYSL3, PGR, YWHAZ, MARCKS, SEMA3A, PIR, EREG and combinations thereof; and wherein said expression level is predicative of oocyte competency.

6. The method of claim 5, wherein assessing expression of said at least one follicular cell marker comprises measuring polynucleotide and/or polypeptide expression levels for said marker.

7. The method of claim 6, comprising measuring DNA and/or mRNA levels of a polynucleotide encoding said at least one follicular cell marker.

8. The method of claim 6, wherein said polynucleotide comprises a sequence as set forth in GenBank™ or Unigene™ for the accession numbers provided in Tables 2A and 2B.

9. The method of claim 6, comprising measuring expression levels of a polypeptide encoded by said at least one follicular cell marker, wherein said polypeptide comprises an amino acid sequence as set forth in GenBank™ or Unigene™ for the accession numbers provided in Tables 2A and 2B.

10. The method of claim 5, comprising assessing expression of at least two follicular cell markers.

11. The method of claim 5, further comprising the step of comparing the expression level of said at least one marker with a control expression level.

12. The method of claim 11, wherein the control expression level is derived from an expression level measured from a control group consisting of: follicular cells from one or from a pool of follicles comprising oocyte(s) competent for fertilization; follicular cells from one or from a pool of follicles comprising oocyte(s) not competent for fertilization; follicular cells from one or from a pool of follicles comprising oocyte(s) competent for embryo development; and follicular cells from one or from a pool of follicles comprising oocyte(s) not competent for embryo development.

13. The method of claim 5, wherein said follicular cells are obtained before ovulation by aspirating the oocyte in said ovarian follicle.

14. A method of evaluating competence of a mammalian oocyte, said method comprising:
(a) assessing in follicular cells originating from an ovarian follicle comprising said oocyte an expression level of at least one polynucleotide, wherein said at least one polynucleotide comprises a nucleotide sequence for UGP2; and
(b) comparing the expression level of said at least one polynucleotide with a control expression level;
wherein a differential between expression level of said at least one polynucleotide and the control expression level is predicative of oocyte competency.

15. A method for evaluating competence of a mammalian oocyte, said method comprising:
(a) assessing in follicular cells originating from an ovarian follicle comprising said oocyte an expression level of at least one polypeptide, wherein said polypeptide comprises an amino acid sequence for UGP2; and
(b) comparing the expression level of said at least one polypeptide with a control expression level;
wherein a differential between expression level of said at least one polypeptide and the control expression level is predicative of oocyte competency.

16. A method for selecting a mammalian oocyte for assisted reproduction (AR), the method comprising:
obtaining mammalian follicular cells of an ovarian follicle which contains said oocyte;
determining expression level of at least one follicular cell marker, wherein said at least one follicular cell marker is selected from the group consisting of UGP2, PHLDA1, GAPBP1, SFRP1, HOMER1, LRP8, DPYSL3, PGR, YWHAZ, MARCKS, SEMA3A, PIR, EREG and combinations thereof;
comparing the expression level of said at least one marker with a control expression level in control follicular cells; and
selecting for AR an oocyte which follicular cells have a desirable expression level of said at least one marker when compared with the control expression level.

17. A method for screening a compound stimulatory or inhibitory to mammalian oocyte competence, said method comprising the steps of:
a) contacting follicular cells with a compound to be screened for activity to stimulate or inhibit the competence of an oocyte;
b) determining an expression level of at least one follicular cell marker in follicular cells contacted with said compound, wherein said at least one follicular cell marker is selected from the group consisting of UGP2, PHLDA1, GAPBP1, SFRP1, HOMER1, LRP8, DPYSL3, PGR, YWHAZ, MARCKS, SEMA3A, PIR, EREG and combinations thereof;
c) comparing the expression level measured in step b) with the expression level of non-contacted follicular cells;
wherein a difference in said expression levels is indicative of the compound stimulatory or inhibitory effect.

18. (canceled)

19. The method of claim 17, wherein said contacting is carried out in vivo.

20-56. (canceled)

57. The method of claim 10, wherein said at least two follicular cell markers comprises UGP2 and at least one of PHLDA1, GAPBP1, SFRP1, HOMER1, LRP8, DPYSL3, PGR, YWHAZ, MARCKS, SEMA3A, PIR, EREG.

58. The method of claim 57, comprising assessing expression of at least three follicular cell markers comprising UGP2, PHLDA1, and GAPBP 1.


SEQUENCE LISTING (112 sequences)

SEQ ID NO: 1 - DNA, 4674 bp, Homo sapiens
  CDS (254)..(3451)
acaccctaatgcctccaacaataactgttgactttttattttcagtcagagaagcctggcaaccaagaactgtttttttggtggtttacgagaacttaactgaattggaaaatatttgctttaatgaaacaatttactcttgtgcaacactaaattgtgtcaatcaagcaaataaggaagaaagtcttatttataaaattgcctgctcctgattttacttcatttcttctcaggctccaagaaggggaaaaaaatgaagattttgatacttggtatttttctgtttttatgtagtaccccagcctgggcgaaagaaaagcattattacattggaattattgaaacgacttgggattatgcctctgaccatggggaaaagaaacttatttctgttgacacggaacattccaatatctatcttcaaaatggcccagatagaattgggagactatataagaaggccctttatcttcagtacacagatgaaacctttaggacaactatagaaaaaccggtctggcttgggtttttaggccctattatcaaagctgaaactggagataaagtttatgtacacttaaaaaaccttgcctctaggccctacacctttcattcacatggaataacttactataaggaacatgagggggccatctaccctgataacaccacagattttcaaagagcagatgacaaagtatatccaggagagcagtatacatacatgttgcttgccactgaagaacaaagtcctggggaaggagatggcaattgtgtgactaggatttaccattcccacattgatgctccaaaagatattgcctcaggactcatcggacctttaataatctgtaaaaaagattctctagataaagaaaaagaaaaacatattgaccgagaatttgtggtgatgttttctgtggtggatgaaaatttcagctggtacctagaagacaacattaaaacctactgctcagaaccagagaaagttgacaaagacaacgaagacttccaggagagtaacagaatgtattctgtgaatggatacacttttggaagtctcccaggactctccatgtgtgctgaagacagagtaaaatggtacctttttggtatgggtaatgaagttgatgtgcacgcagctttctttcacgggcaagcactgactaacaagaactaccgtattgacacaatcaacctctttcctgctaccctgtttgatgcttatatggtggcccagaaccctggagaatggatgctcagctgtcagaatctaaaccatctgaaagccggtttgcaagcctttttccaggtccaggagtgtaacaagtcttcatcaaaggataatatccgtgggaagcatgttagacactactacattgccgctgaggaaatcatctggaactatgctccctctggtatagacatcttcactaaagaaaacttaacagcacctggaagtgactcagcggtgttttttgaacaaggtaccacaagaattggaggctcttataaaaagctggtttatcgtgagtacacagatgcctccttcacaaatcgaaaggagagaggccctgaagaagagcatcttggcatcctgggtcctgtcatttgggcagaggtgggagacaccatcagagtaaccttccataacaaaggagcatatcccctcagtattgagccgattggggtgagattcaataagaacaacgagggcacatactattccccaaattacaacccccagagcagaagtgtgcctccttcagcctcccatgtggcacccacagaaacattcacctatgaatggactgtccccaaagaagtaggacccactaatgcagatcctgtgtgtctagctaagatgtattattctgctgtggatcccactaaagatatattcactgggcttattgggccaatgaaaatatgcaagaaaggaagtttacatgcaaatgggagacagaaagatgtagacaaggaattctatttgtttcctacagtatttgatgagaatgagagtttactcctggaagataatattagaatgtttacaactgcacctgatcaggtggataaggaagatgaagactttcaggaatctaataaaatgcactccatgaatggattcatgtatgggaatcagccgggtctcactatgtgcaaaggagattcggtcgtgtggtacttattcagcgccggaaatgaggccgatgtacatggaatatacttttcaggaaacacatatctgtggagaggagaacggagagacacagcaaacctcttccctcaaacaagtcttacgctccacatgtggcctgacacagaggggacttttaatgttgaatgccttacaactgatcattacacaggcggcatgaagcaaaaatatactgtgaaccaatgcaggcggcagtctgaggattccaccttctacctgggagagaggacatactatatcgcagcagtggaggtggaatgggattattccccacaaagggagtgggaaaaggagctgcatcatttacaagagcagaatgtttcaaatgcatttttagataagggagagttttacataggctcaaagtacaagaaagttgtgtatcggcagtatactgatagcacattccgtgttccagtggagagaaaagctgaagaagaacatctgggaattctaggtccacaacttcatgcagatgttggagacaaagtcaaaattatctttaaaaacatggccacaaggccctactcaatacatgcccatggggtacaaacagagagttctacagttactccaacattaccaggtgaaactctcacttacgtatggaaaatcccagaaagatctggagctggaacagaggattctgcttgtattccatgggcttattattcaactgtggatcaagttaaggacctctacagtggattaattggccccctgattgtttgtcgaagaccttacttgaaagtattcaatcccagaaggaaactggaatttgcccttctgtttctagtttttgatgagaatgaatcttggtacttagatgacaacatcaaaacatactctgatcaccccgagaaagtaaacaaagatgatgaggaattcatagaaagcaataaaatgcatgctattaatggaagaatgtttggaaacctacaaggcctcacaatgcacgtgggagatgaagtcaactggtatctgatgggaatgggcaatgaaatagacttacacactgtacattttcacggccatagcttccaatacaagcacaggggagtttatagttctgatgtctttgacattttccctggaacataccaaaccctagaaatgtttccaagaacacctggaatttggttactccactgccatgtgaccgaccacattcatgctggaatggaaaccacttacaccgttctacaaaatgaagacaccaaatctggctgaatgaaataaattggtgataagtggaaaaaagagaaaaaccaatgattcataacaatgtatgtgaaagtgtaaaatagaatgttactttggaatgactataaacattaaaagaagactggaagcatacaactttgtacatttgtgggggaaaactattaattttttgcaaatggaaagatcaacagactatataatgatacatgactgacacttgtacactaggtaataaaactgattcatacagtctaatgatatcaccgctgttagggttttataaaactgcatttaaaaaaagatctatgaccagatattctcctgggtgctcctcaaaggaacactattaaggttcattgaaatgttttcaatcattgccttcccattgatccttctaacatgctgttgacatcacacctaatattcagagggaatgggcaaggtatgagggaaggaaataaaaaataaaataaataaaatagaatgacacaaatttgagttttgtgaacccctgaacagatggtcttaaggacgttatctggaactggagaaaagcagagttgagagacaattctatagattaaatcctggtaaggacaaacattgccattagaagaaaagcttcaaaatagacctgtggcagatgtcacatgagtagaatttctgcccagccttaactgcattcagaggataatatcaatgaactaaacttgaactaaaaattttttaaacaaaaagttataaatgaagacacatggttgtgaatacaatgatgtatttctttattttcacatacactctagctaaaagagcaagagtacacatcaacaaaaatggaaacaaggctttggctgaaaaaaacatgcatttgacaaatcatgttaatagctagacaagaagaaagttagctttgtaaacttctacttcatttgattcagagaaacagagcatgagttttcttaaaagtaacaagaaaaggaacaaaaaaaatgaggtttgaaatcttttaccatggcaaaacattaacatctttctcaaaaacatagagaaatctggaaaaatcaagaagataaaattctggaccagttagtgacattctttcaagcatacttgtaaaatgtttccttaaagtgttcttgggatgaaaatgattgtcatgtctccaacaacagtgaactgatgttgttccttggaataaaagtcaatccccaccttaaaaaaaaaaaaaa

SEQ ID NO: 2 - protein, 1065 aa, Homo sapiens
MKILILGIFLFLCSTPAWAKEKHYYIGIIETTWDYASDHGEKKLISVDTEHSNIYLQNGPDRIGRLYKKALYLQYTDETFRTTIEKPVWLGFLGPIIKAETGDKVYVHLKNLASRPYTFHSHGITYYKEHEGAIYPDNTTDFQRADDKVYPGEQYTYMLLATEEQSPGEGDGNCVTRIYHSHIDAPKDIASGLIGPLIICKKDSLDKEKEKHIDREFVVMFSVVDENFSWYLEDNIKTYCSEPEKVDKDNEDFQESNRMYSVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSVPPSASHVAPTETFTYEWTVPKEVGPTNADPVCLAKMYYSAVDPTKDIFTGLIGPMKICKKGSLHANGRQKDVDKEFYLFPTVFDENESLLLEDNIRMFTTAPDQVDKEDEDFQESNKMHSMNGFMYGNQPGLTMCKGDSVVWYLFSAGNEADVHGIYFSGNTYLWRGERRDTANLFPQTSLTLHMWPDTEGTFNVECLTTDHYTGGMKQKYTVNQCRRQSEDSTFYLGERTYYIAAVEVEWDYSPQREWEKELHHLQEQNVSNAFLDKGEFYIGSKYKKVVYRQYTDSTFRVPVERKAEEEHLGILGPQLHADVGDKVKIIFKNMATRPYSIHAHGVQTESSTVTPTLPGETLTYVWKIPERSGAGTEDSACIPWAYYSTVDQVKDLYSGLIGPLIVCRRPYLKVFNPRRKLEFALLFLVFDENESWYLDDNIKTYSDHPEKVNKDDEEFIESNKMHAINGRMFGNLQGLTMHVGDEVNWYLMGMGNEIDLHTVHFHGHSFQYKHRGVYSSDVFDIFPGTYQTLEMFPRTPGIWLLHCHVTDHIHAGMETTYTVLQNEDTKSG

SEQ ID NO: 3 - DNA, 1852 bp, Homo sapiens
  CDS (85)..(1212)
accgccgagaccgcgtccgccccgcgagcacagagcctcgcctttgccgatccgccgcccgtccacacccgccgccagctcaccatggatgatgatatcgccgcgctcgtcgtcgacaacggctccggcatgtgcaaggccggcttcgcgggcgacgatgccccccgggccgtcttcccctccatcgtggggcgccccaggcaccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttctaggcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcgttgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttcccccttttttgtcccccaacttgagatgtatgaaggcttttggtctccctgggagtgggtggaggcagccagggcttacctgtacactgacttgagaccagttgaataaaagtgcacaccttaaaaatgaaaaaaaaaaaaaaaaaaaaaaaaaaaaaaaaaaaaaaaaa

SEQ ID NO: 4 - protein, 375 aa, Homo sapiens
MDDDIAALVVDNGSGMCKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF

SEQ ID NO: 5 - DNA, 1460 bp, Homo sapiens
  CDS (105)..(1292)
tgcagcgcaggtgagctctcctgaggacctctctgtcagctcccctgattgtagggaggatccagtgtggcaagaaactcctccagcccagcaagcagctcaggatgttcctgaaggccgtggtcctgaccctggccctggtggctgtcgccggagccagggctgaggtcagtgctgaccaggtggccacggtgatgtgggactacttcagccagctgagcaacaatgccaaggaggccgtggaacatctccagaaatctgaactcacccagcaactcaatgccctcttccaggacaaacttggagaagtgaacacttacgcaggtgacctgcagaagaagctggtgccctttgccaccgagctgcatgaacgcctggccaaggactcggagaaactgaaggaggagattgggaaggagctggaggagctgagggcccggctgctgccccatgccaatgaggtgagccagaagatcggggacaacctgcgagagcttcagcagcgcctggagccctacgcggaccagctgcgcacccaggtcagcacgcaggccgagcagctgcggcgccagctgaccccctacgcacagcgcatggagagagtgctgcgggagaacgccgacagcctgcaggcctcgctgaggccccacgccgacgagctcaaggccaagatcgaccagaacgtggaggagctcaagggacgccttacgccctacgctgacgaattcaaagtcaagattgaccagaccgtggaggagctgcgccgcagcctggctccctatgctcaggacacgcaggagaagctcaaccaccagcttgagggcctgaccttccagatgaagaagaacgccgaggagctcaaggccaggatctcggccagtgccgaggagctgcggcagaggctggcgcccttggccgaggacgtgcgtggcaacctgaggggcaacaccgaggggctgcagaagtcactggcagagctgggtgggcacctggaccagcaggtggaggagttccgacgccgggtggagccctacggggaaaacttcaacaaagccctggtgcagcagatggaacagctcaggcagaaactgggcccccatgcgggggacgtggaaggccacttgagcttcctggagaaggacctgagggacaaggtcaactccttcttcagcaccttcaaggagaaagagagccaggacaagactctctccctccctgagctggagcaacagcaggaacagcagcaggagcagcagcaggagcaggtgcagatgctggcccctttggagagctgagctgcccctggtgcactggccccaccctcgtggacacctgccctgccctgccacctgtctgtctgtctgtcccaaagaagttctggtatgaacttgaggacacatgtccagtgggaggtgagaccacctctcaatattcaataaagctgctgagaatctagcctc

SEQ ID NO: 6 - protein, 396 aa, Homo sapiens
MFLKAVVLTLALVAVAGARAEVSADQVATVMWDYFSQLSNNAKEAVEHLQKSELTQQLNALFQDKLGEVNTYAGDLQKKLVPFATELHERLAKDSEKLKEEIGKELEELRARLLPHANEVSQKIGDNLRELQQRLEPYADQLRTQVSTQAEQLRRQLTPYAQRMERVLRENADSLQASLRPHADELKAKIDQNVEELKGRLTPYADEFKVKIDQTVEELRRSLAPYAQDTQEKLNHQLEGLTFQMKKNAEELKARISASAEELRQRLAPLAEDVRGNLRGNTEGLQKSLAELGGHLDQQVEEFRRRVEPYGENFNKALVQQMEQLRQKLGPHAGDVEGHLSFLEKDLRDKVNSFFSTFKEKESQDKTLSLPELEQQQEQQQEQQQEQVQMLAPLES

SEQ ID NO: 7 - DNA, 60 bp, Homo sapiens
aatgtggtgccatgcaggggtctacggtttgtggtagtaatctaataccttaaccccaca

SEQ ID NO: 8 - DNA, 60 bp, Homo sapiens
tccgggataacatccagggcattacaaaaccggctattcgccgtttggctcggcgcggtg

SEQ ID NO: 9 - DNA, 60 bp, Homo sapiens
tcttggttgatcctcttgtactgggagaggtgccttttgtatccccaattaaaggtagaa

SEQ ID NO: 10 - DNA, 333 bp, Homo sapiens
ctagcgcctcgtaaggtgctccgggataactccagggcattacaaaaccggctatccgccgtttggctcggcgcggtggcgtcaagcgcatttccggtcttatctatgaggagactcgaggtgtgcttaaggttttcttagagaacgttattcgagacgccgtcacctatacggagcacgccaagcgcaaaactgtcacagccatggatgtagtatatgccctaaaacgtcaggggcgcactctgtatggcttcggcggctgaatctaagaatacgcggtctcctgagaacttcaaaaaacaaaaaaaaaaaaaaaaaaaaaaaaaatccccc

SEQ ID NO: 11 - DNA, 278 bp, Homo sapiens
tagggtttacgacctcgatgttggatcaggacatcccgatggtgcagccgctattaaaggttcgtttgttcaacgattaaagtcctacgtgatctgagttcagaccggagtaatccaggtcggtttctatctacttcaaattcctcccagtacctcggcgggtcagtgatacatttggctgactctggttcagtattctcttaggtgattatattctctcatgtacagttacaggaaattaaaatgttaaagtaacctaaaatgaattcagaccaata

SEQ ID NO: 12 - DNA, 752 bp, Homo sapiens
tctggttggtttctactatttcattcactttatctattttgcagtgtagtctcccggcagcaataaacctggacagttcctgatcaatgaattccacaccaacaccaaacgcttctgccatatagtcaagggttaatgacctatatgattccagcagctgactgtatgcatgaattctcatttctcttacatagtatcgataatgaggggcaaaaagccagtcctttttcatttcctgttccacaaccgctaatgattggaagaaaacagagtaacggcattcatagagtgaaaacagatactgccgaactgctggaagactgtgcaacacttcaagaatctctgctcctttaatgaccttttccctgagatctggtctttctaaggcaatcatactgacatagacagtataagtcacaaatgttttataatccatgagttcataggatgtaaatgttgaaacagtgtcaaggaagagttcagctgcctgtttgaaatcacgaatagccacacaataaagaccctgatacacttttaggcggtttctcctgtcccagtctcctccttcttctattaagcttttggccttttctgtgtttcgtgtgatgagatcattatccatataaaataagccaatcctaaggagatagaatacaatatccaatcggtgacccagggccacagttttgtcatatgtcttgcgaaaggctgtcagagctccctctttgtcacctatccggcagaggtacctc

SEQ ID NO: 13 - DNA, 300 bp, Homo sapiens
gctggcctcatttgtgtccggagctcaggagttccaaaccgactcagtcgcaccaagtttccgtcttttggaattggggaaggagtttctttctttcttttcttttttcttgagccagttttaatcgctttgaataaatactcccttaagtagttaaatataggaggagaaagaatacatcggttgttaaagcaggagaggaagagagacctgccctgtagcgtgactcctctagaaaaaaaaaaaaaaagcgggagttttttacaaacccccaaaaaggtcaaaatttgtcccccggcc

SEQ ID NO: 14 - DNA, 596 bp, Homo sapiens
tcagcttcatggaaaaggaaaaatgcatttttcaaccgaaagggcagatccaatagaagacccgctccttaaataaacataaaatgtaaaaagttggaaaattaagagtaatgttccatctggaaactgaacttttgtccttgaacttgtgttggcaccaagcctcatacacagtgagctcaataactgttgggacaaaggaaggaaggacaaaatgtgtaacttcccagcatctgggagatgctgtctcttgcctcactgagtgttccttttctttgctctcatgtcattccctgagaacaatgaattctgggacaggctaaacatcatgatgaagtttcttaaacagactttcttagtggaaatccatttagatctgggtgtgctctatggggagtgctgacgtcaaagagcaaatgtctataaggggcccttttaaaatgaacattttcctcattgagcaagctgggattctctaatgtagaaatcaagccatctttataatttcacttcagatgtttatgtttttgttttttttgtctccaatgatggtaaaaataaaaactacgcattactgcaaaaaaaaaaaaaaaaaa

SEQ ID NO: 15 - DNA, 549 bp, Homo sapiens
ggggattgggcggaaagtgagagccagcagcaaaaactacattttgcaacttgttggtgtggatctattggctgatctatgcctttcaactagaaaattctaatgattggcaagtcacgttgttttcaggtccagagtagtttctttctgtctgctttaaatggaaacagactcataccacacttacaattaaggtcaagcccagaaagtgataagtgcagggaggaaaagtgcaagtccattatgtaatagtgacagcaaagggaccaggggagaggcattgccttctctgcccacagtctttccgtgtgattgtctttgaatctgaatcagccagtctcagatgccccaaagtttcggttcctatgagcccggggcatgatctgatccccaagacatgtggaggggcagcctgtgcctgcctttgtgtcagaaaaaggaaaccacagtgagcctgagagagacggcgattttcgggctgagaaggcagtagttttcaaaacacatagtcaaaaaaaaaaaaaaaaaaaaaaaaaaaacccccccccc

SEQ ID NO: 16 - DNA, 515 bp, Homo sapiens
gacggtctggtctagcaatgttaagtatatttacagaatatgcagttacatttatttatatattttgcaagaaatcttttctgaatgattaatgcatttcaatttacaaatactaatggttgttggggaactgtttattatagatcattttaaggtgtatagctattttaaaggcgatccatttccatcaagcagccaatcagaggactattgggattggagccgtttactctgtctgggtcttcacaccaaccacatctctagtcaatcctcataaggcaatattctaaactgttaactaggcatttcaaaagaggaattcaattcagtgggctctcctcagtgaacaggttttaatgttattttgatgtaattttaaaggacttttagcaaacatgcatttctttatatatttcttttaaggagctattttaaaaagtaagtcaagtgctgtctagtctgcttatggagtaagaattgcatcagagtccatatattcttgctgtacctcggcc

SEQ ID NO: 17 - DNA, 427 bp, Homo sapiens
atgcatcacttgcataggtattatttttcccacaaagatgatgaaagttactaggaaagaaagaaacccttttattctgtaggtccttgaaactaagtaagctagcagctggttttattggaatgacaatgttcttggaaggagcagcttacaagatatcttgaattttcaaactgcaaaatttatgcttttttgaaaatttcgtagtggggatgtgaaactcttctgtgccttccatcatgatttccacatgaaagcactttaaggcactggattttaagataatgtttttggaaaactcaaaacatgggtttctgaaatacttcatggactcatttcccccccaggaaattattcttacagaaaagaaaaaaactgcttttgtatgtagaacaagaactttttgtacctcggccgcgaccacgct

SEQ ID NO: 18 - DNA, 22 bp, Artificial Sequence
  Sequence is a chemically synthesized primer or probe
ctgggtttccatcttctacagg

SEQ ID NO: 19 - DNA, 22 bp, Artificial Sequence
  Sequence is a chemically synthesized primer or probe
gccattcatgtcagagctacac

SEQ ID NO: 20 - DNA, 22 bp, Artificial Sequence
  Sequence is a chemically synthesized primer or probe
caaagtcagtcttgagcagagg

SEQ ID NO: 21 - DNA, 22 bp, Artificial Sequence
  Sequence is a chemically synthesized primer or probe
agaagcagcatgatagggaaag

SEQ ID NO: 22 - DNA, 20 bp, Artificial Sequence
  Sequence is a chemically synthesized primer or probe
gattcagaagccagccagag

SEQ ID NO: 23 - DNA, 22 bp, Artificial Sequence
  Sequence is a chemically synthesized primer or probe
taccttccattgccctcttaaa

SEQ ID NO: 24 - DNA, 22 bp, Artificial Sequence
  Sequence is a chemically synthesized primer or probe
ggtgatgacaagaaagggattg

SEQ ID NO: 25 - DNA, 22 bp, Artificial Sequence
  Sequence is a chemically synthesized primer or probe
gcgtgctgtctttgtatgactc

SEQ ID NO: 26 - DNA, 22 bp, Artificial Sequence
  Sequence is a chemically synthesized primer or probe
ttcttcctctgccttgtttctc

SEQ ID NO: 27 - DNA, 23 bp, Artificial Sequence
  Sequence is a chemically synthesized primer or probe
cagcctttacaccatttctagtg

SEQ ID NO: 28 - DNA, 21 bp, Artificial Sequence
  Sequence is a chemically synthesized primer or probe
gactcagtcgcaccaagtttc

SEQ ID NO: 29 - DNA, 22 bp, Artificial Sequence
  Sequence is a chemically synthesized primer or probe
ttcctctcctgctttaacaacc

SEQ ID NO: 30 - DNA, 22 bp, Artificial Sequence
  Sequence is a chemically synthesized primer or probe
cagccctgaagagagaatcatc

SEQ ID NO: 31 - DNA, 22 bp, Artificial Sequence
  Sequence is a chemically synthesized primer or probe
gctctgtgtcaatgacttccag

SEQ ID NO: 32 - DNA, 22 bp, Artificial Sequence
  Sequence is a chemically synthesized primer or probe
ccagagtgctcagaaactcaag

SEQ ID NO: 33 - DNA, 23 bp, Artificial Sequence
  Sequence is a chemically synthesized primer or probe
ccatctttatcttcatccacagc

SEQ ID NO: 34 - DNA, 22 bp, Artificial Sequence
  Sequence is a chemically synthesized primer or probe
aacccagtaaggatggtgtgac

SEQ ID NO: 35 - DNA, 22 bp, Artificial Sequence
  Sequence is a chemically synthesized primer or probe
aagacaaagtggcttctcttgg

SEQ ID NO: 36 - DNA, 24 bp, Artificial Sequence
  Sequence is a chemically synthesized primer or probe
agctcaataactgttgggacaaag

SEQ ID NO: 37 - DNA, 24 bp, Artificial Sequence
  Sequence is a chemically synthesized primer or probe
catgtgagggaaactcctttaagt

SEQ ID NO: 38 - DNA, 22 bp, Artificial Sequence
  Sequence is a chemically synthesized primer or probe
ttctaatgattggcaagtcacg

SEQ ID NO: 39 - DNA, 22 bp, Artificial Sequence
  Sequence is a chemically synthesized primer or probe
tggctgattcagattcaaagac

SEQ ID NO: 40 - DNA, 22 bp, Artificial Sequence
  Sequence is a chemically synthesized primer or probe
gggaacaacctatcttcagcac

SEQ ID NO: 41 - DNA, 22 bp, Artificial Sequence
  Sequence is a chemically synthesized primer or probe
attgcctttgagccatctaaac

SEQ ID NO: 42 - DNA, 22 bp, Artificial Sequence
  Sequence is a chemically synthesized primer or probe
tcccacagaaatgatgaaagtg

SEQ ID NO: 43 - DNA, 22 bp, Artificial Sequence
  Sequence is a chemically synthesized primer or probe
tgatggaaggcacagaatacag

SEQ ID NO: 44 - DNA, 20 bp, Artificial Sequence
  Sequence is a chemically synthesized primer or probe
accacagtccatgccatcac

SEQ ID NO: 45 - DNA, 20 bp, Artificial Sequence
  Sequence is a chemically synthesized primer or probe
tccaccaccctgttgctgta

SEQ ID NO: 46 - DNA, 24 bp, Artificial Sequence
  Sequence is a chemically synthesized primer or probe
cgtgacattaaggagaagctgtgc

SEQ ID NO: 47 - DNA, 24 bp, Artificial Sequence
  Sequence is a chemically synthesized primer or probe
ctcaggaggagcaatgatcttgat

SEQ ID NO: 48 - DNA, 20 bp, Artificial Sequence
  Sequence is a chemically synthesized primer or probe
cagcaggcagagaaaatcct

SEQ ID NO: 49 - DNA, 20 bp, Artificial Sequence
  Sequence is a chemically synthesized primer or probe
gcatctttcgggttcctttt

SEQ ID NO: 50 - DNA, 23 bp, Artificial Sequence
  Sequence is a chemically synthesized primer or probe
acaaggatggagacctcattgcc

SEQ ID NO: 51 - DNA, 22 bp, Artificial Sequence
  Sequence is a chemically synthesized primer or probe
tgctctcgctctgtctttaccc

SEQ ID NO: 52 - DNA, 24 bp, Artificial Sequence
  Sequence is a chemically synthesized primer or probe
gctgcctgctccaagtgatttcaa

SEQ ID NO: 53 - DNA, 23 bp, Artificial Sequence
  Sequence is a chemically synthesized primer or probe
gcaagaatgtgagtagccacggt

SEQ ID NO: 54 - DNA, 18 bp, Artificial Sequence
  Sequence is a chemically synthesized primer or probe
ccatcgtaaggtgctccg

SEQ ID NO: 55 - DNA, 20 bp, Artificial Sequence
  Sequence is a chemically synthesized primer or probe
ttggcgtgctccgtataggt

SEQ ID NO: 56 - DNA, 24 bp, Artificial Sequence
  Sequence is a chemically synthesized primer or probe
acaagcctgatactcctcagtcag

SEQ ID NO: 57 - DNA, 24 bp, Artificial Sequence
  Sequence is a chemically synthesized primer or probe
gcaacccaagaactacacaagcag

SEQ ID NO: 58 - DNA, 24 bp, Artificial Sequence
  Sequence is a chemically synthesized primer or probe
ttccagcagttcggcagtatctgt

SEQ ID NO: 59 - DNA, 24 bp, Artificial Sequence
  Sequence is a chemically synthesized primer or probe
ttccacaccaacaccaaacgcttc

SEQ ID NO: 60 - DNA, 24 bp, Artificial Sequence
  Sequence is a chemically synthesized primer or probe
tgaagggacgcacagttatcgtga

SEQ ID NO: 61 - DNA, 24 bp, Artificial Sequence
  Sequence is a chemically synthesized primer or probe
aagcaacacctggtctcatccgaa

SEQ ID NO: 62 - DNA, 24 bp, Artificial Sequence
  Sequence is a chemically synthesized primer or probe
accctgtgtttgacgagaccttca

SEQ ID NO: 63 - DNA, 24 bp, Artificial Sequence
  Sequence is a chemically synthesized primer or probe
catcttcggcaagtgtctggcttt

SEQ ID NO: 64 - DNA, 24 bp, Artificial Sequence
  Sequence is a chemically synthesized primer or probe
ggtaatctttcaggaaggtggaga

SEQ ID NO: 65 - DNA, 22 bp, Artificial Sequence
  Sequence is a chemically synthesized primer or probe
gctgatgtcatcccagactttg

SEQ ID NO: 66 - DNA, 24 bp, Artificial Sequence
  Sequence is a chemically synthesized primer or probe
caatctcaacaatgtgttcctgcg

SEQ ID NO: 67 - DNA, 22 bp, Artificial Sequence
  Sequence is a chemically synthesized primer or probe
tacctctttccgttggtcagcc

SEQ ID NO: 68 - DNA, 23 bp, Artificial Sequence
  Sequence is a chemically synthesized primer or probe
cttcagatcagcaggacagttgg

SEQ ID NO: 69 - DNA, 24 bp, Artificial Sequence
  Sequence is a chemically synthesized primer or probe
gggagttgttacaagatggaacgg

SEQ ID NO: 70 - DNA, 21 bp, Artificial Sequence
  Sequence is a chemically synthesized primer or probe
aaccctatttccccaccccag

SEQ ID NO: 71 - DNA, 22 bp, Artificial Sequence
  Sequence is a chemically synthesized primer or probe
gctctctaaacttcccgtggca

SEQ ID NO: 72 - DNA, 22 bp, Artificial Sequence
  Sequence is a chemically synthesized primer or probe
tacttcgtgtgctccgacccat

SEQ ID NO: 73 - DNA, 22 bp, Artificial Sequence
  Sequence is a chemically synthesized primer or probe
agtccacagccacagcctactc

SEQ ID NO: 74 - DNA, 22 bp, Artificial Sequence
  Sequence is a chemically synthesized primer or probe
acctgaaacccagtgcccagaa

SEQ ID NO: 75 - DNA, 22 bp, Artificial Sequence
  Sequence is a chemically synthesized primer or probe
tgttgttgcggttgtcagcagt

SEQ ID NO: 76 - DNA, 22 bp, Artificial Sequence
  Sequence is a chemically synthesized primer or probe
accagcctcgtcaatttcccaa

SEQ ID NO: 77 - DNA, 23 bp, Artificial Sequence
  Sequence is a chemically synthesized primer or probe
catctcctgcaagtctcgttcca

SEQ ID NO: 78 - DNA, 21 bp, Artificial Sequence
  Sequence is a chemically synthesized primer or probe
gcagcgatttgccttgacaac

SEQ ID NO: 79 - DNA, 22 bp, Artificial Sequence
  Sequence is a chemically synthesized primer or probe
tttaagctcagggtgctcgtcc

SEQ ID NO: 80 - DNA, 21 bp, Artificial Sequence
  Sequence is a chemically synthesized primer or probe
caactggaccagctccttcac

SEQ ID NO: 81 - DNA, 24 bp, Artificial Sequence
  Sequence is a chemically synthesized primer or probe
gcacagtcattgtcataagcatgt

SEQ ID NO: 82 - DNA, 25 bp, Artificial Sequence
  Sequence is a chemically synthesized primer or probe
cagtgatacatttggctgactctgg

SEQ ID NO: 83 - DNA, 24 bp, Artificial Sequence
  Sequence is a chemically synthesized primer or probe
gcctccaccgtaacctttgtactt

SEQ ID NO: 84 - DNA, 24 bp, Artificial Sequence
  Sequence is a chemically synthesized primer or probe
tgctggacccaacacaaatggttc

SEQ ID NO: 85 - DNA, 24 bp, Artificial Sequence
  Sequence is a chemically synthesized primer or probe
tggtgatcttcttgctggtcttgc

SEQ ID NO: 86 - DNA, 24 bp, Artificial Sequence
  Sequence is a chemically synthesized primer or probe
cgtgacattaaggagaagctgtgc

SEQ ID NO: 87 - DNA, 24 bp, Artificial Sequence
  Sequence is a chemically synthesized primer
ctcaggaggagcaatgatcttgat

SEQ ID NO: 88 - DNA, 250 bp, Homo sapiens
tactatactcaattgatccaataacttgaccaacggaacaagttaccctagggataacagcgcaatcctattctagagtccatatcaacaatagggtttacgacctcgatgttggatcaggacatcccgatggtgcagccgctattaaaggttcgtttgttcaacgattaaagtcctacgtgatctgagttcagaccggagtaatccaggtcggtttctatctacttcaaattcctcccagtacctcggc

SEQ ID NO: 89 - DNA, 418 bp, Homo sapiens
ttcttcttgtattgatccttttatcgttatataggtatccttccttatttttctttacagcctttagtttaaaatctattttgtctcatatgactattgctacccccatttccttgccatttgcatttgcttgaaatatctgtttccatctcctcactttcagtctatgtgtgtcctttgccctaaagtgggtctcttgtaggcaggctctttttttttaatccaacctgccattccgtgtcttttactgggagcatttagttcattaacatttaaggtaatcattgatagatatgtattcatcaccattttaaatcttaatttttccaactgactttgtatttctttgttcctttctgttttttttctttgtggtttgattgtttccctttatattatgcttgtacctcggccgcga

SEQ ID NO: 90 - DNA, 327 bp, Homo sapiens
tttgggggggggggttccaaaaaaaaaaattttttggggggaaaaaaaaaaaaaattttttaaaattttttgggggttttttttccccccccccccccaaaaggattttttttaaaaaaaaaaatttttttttaaaaaacctttttttttttgggggttaaaagggggaaaaaggcccccccaaaaaaaaagggggggggggccccccaggggggggggggggggccccccctttttttttttgggggggggaaaaaaaaatttttaggggggcaaaaattttttttttttttttttaaaggggaaaaacccccgggcccggggggg

SEQ ID NO: 91 - DNA, 245 bp, Homo sapiens
aaacactgaactgacaattaacagcccaatatctacaatcaaccaacaagtcattattaccctcactgtcaacccaacacaggcatgctcataaggaaaggttaaaaaaagtaaaaggaactcggcaaatcttaccccgcctgtttaccaaaaacatcacctctagcatcaccagtattagaggcaccgcctgcccagtgacacatgtttaacggccgcggtacctgcccgggcggccgctcgaa

SEQ ID NO: 92 - DNA, 602 bp, Homo sapiens
ccccccccccccgggggaaaaaaaaccccccccccccccccccaaggggggggggaaaaggggcccccccccccccccgggggaatttttttttttttttaaaaaaaagggggttttaaaattttgggccggggggggcacaacccccaaacccggggacccacccccctggccccccaaaaaaaaaaagattaaaaacccctgctaaaggaaacccacccggaacccgggggccccccccccaaaaattaaaagggaaaggaaaccgcccacctttttttttcggggttgttttttttaaaaggttttttttttttttaaaaaaacccctttaatccgccccccaggggggggggggggggggaaaaaaaaaggggggggccccttaaaacccgggggcaaaaaaaccccttttccccaaaaaaaaaaaaaaaagggcccccccccccccctggggctttttatttttttttggggaaaaaaaaaaaaacccccgcccaaaaaaaattggggtaaaaaaaccaaaagggaatttattttcctttaaaaaaaaggaaaaaattaatttctcccccaaaaaaaaaaaaaaaaaaaaaaaaaaa

SEQ ID NO: 93 - DNA, 402 bp, Homo sapiens
ttctctctgagcttgaggtatactagaagtagagcttctcaaacataatatcattacctcataagcattaacaaatcaggcccaaagagcgtaagtcctagaaatttgttttaaagcagccctagtcatggtgctggtgctaccgccttgttttaggagcctgcctcctgtcagtatgaaaccctcacctgaaaaatgccagcctggacaccaaacactgagccccttcaacaggcacattatttccccctgagatccataagggaatttagtttctactattgtagagttctgaaaagaggtaaaatagtagtcctttggtcatcctatttttgctttcaattttgatatttcagactgtaaaaggccttgggggatgatagtacctcggccgcgaccacg

SEQ ID NO: 94 - DNA, 464 bp, Homo sapiens
tataaagtttggattatacaagtttttcggttatcagaacttttgaattttagaattgtagataaagaattgaggactaaatatgggccaattatttcatattgtattttgggttgagttctacaaagcctcttcaaatgagctatttaaattaaggatatattctttgaacatcaaaagttaaatataaggaagcagtatatttttaaaggattttacatatatgtgtttctgatgaatgtattaactgcattcagttaaagtaaatgaaaataagagtaatctgcagctctgtttaatatatttgagtctttaccttaattttttctttttctaaaattaattgaattcaaaatatctggaaattcctgcaagttgtttttttgaactgcactccagcctgggcgacagagcgagactccgtctcaaagaaaaaaaaaaaaaaaaaaaaaaaaaccccccgc

SEQ ID NO: 95 - DNA, 372 bp, Homo sapiens
atctagatccatgttctcaaacataatcagcttttgaagcatgcagaagctcaggacctactccagacttaccaaatcagaatctgcatttttaaaagatccccacatgaaaaaatgaataaaagatccccaaatgattaacatgcgcattaaattttgagaatattgctctaggctacaaaggagaaatttcttctgtcctaacgatcacatgaagctgcttggtttttgcagttaccactccagttgtaactgtttttccacatagaccttctcatcccttatttccatcttgccctttgtttttccgtgtctctggatcagaaggaaaaattgtatagaaaatgagataatgaaatcccgtacctcggc

SEQ ID NO: 96 - DNA, 367 bp, Homo sapiens
aaatcccaacatacaactgaactcctcacacccaattggaccaatctatcaccctatagaagaactagtgttagtataagtaacatgaaaacattctcctccgcataagcctgcgtcagattaaaacactgaactgacaattaacagcccaatatctacaatcaaccaacaagtcattattaccctcactgtcaacccaacacaggcatgctcataaggaaaggttaaaaaaagtaaaaggaactcggcaaatcttaccccgcctgtttaccaaaaacatcacctctagcatcaccagtattagaggcaccgcctgcccagtgacacatgtttaacggccgcggtacctgcccgggcggccgctcga

SEQ ID NO: 97 - DNA, 462 bp, Homo sapiens
tgcccagcctcagccactattattttctgcctactcagcctacttgacacaacttggatgtctgaagatatatcaaatgaaataagcgccaaagctaaactcttgttcccatctcccctaacccaacagaagtctatttccttctccaataataatggatggcactaccaccaccagtcagtcaactgaaaagcttagatataattcttgagtcctctttctttctcttacaagcccatacccaccccatgtcaatcagggaaaaccttacagatttttcctccaaggtctatcaagaataagcttgtgatccccacctccatgactacatcctagactcagcctccaggatctcttgcctgtggatacaggagcttcctactgtgtctccactgcatgccactcccttccactcctgccacaacagctggtcacaccatcccctatttccccgtacctcgg

SEQ ID NO: 98 - DNA, 650 bp, Homo sapiens
ggcagacgcttcttctacccaccatctcctttttgcttctaaacaatgtttagcacattgttgtagccctaggtagatatgagacttacacataaaccatattttctgtctctctccaccagtattttttcacttcctactattaataatataccttccttggtctttgccataaatgttatgcatatcatatgtcaaaactaatcctagatagaataaagagaaaaatgtaggccgggctcagtggctcacgcctctaatcccagcactttgggaggcagaggcgggtggattcacctgaggttgggagtttgagaccagcctgaccaacatggagaaaccctgtctccactaaaaatacaaaattagccaggcgtggtggtgcatgcctgtaatcccagctactagggaggttgaggcaggagaatcacttgaaccccggaggtggagcttgcagagagctgagatggtgccactgcactcactccagcctgggcaacaagaactaaactctgtctcaaaaaaaaaaaaaatctcccaacaaaaaaaagtccaggatcaggttggcttcattggccatttctatcaaacatttaaaaaaaaattaagggttgggcgcagtggctcacgcctgtaatcctaccacttca

SEQ ID NO: 99 - DNA, 459 bp, Homo sapiens
gggcccccccaaaacgggggagccaaaaggggatttttttggaaaaccgggggggaaaaatttcccaattccctttttttttttttacccttcccttaaaacccccccgggggggggtgaacagggggggaaaaaaaaccttgtgggttaattgaaaaattggggcgttaatttgcccttcccgggtttaaccccccccccggcttaggcggaaaaaaaggtttcctggttacttaaacaaaaattattcccccaaaaggggaaaaaatggccccattggggggggagaattccatttaatttttgggaattttttagggggggggggtggacctgaacccctttctaaatggggggccccttttacccccccaaggggggtaaatttttttccccccccccaagggtttttcccaagggcccaaaaacccccccccccccccccccccccccccccaa

SEQ ID NO: 100 - DNA, 488 bp, Homo sapiens
cttagttgcattgactcacccactcctaaggccaccacatcaaaatctgaggcttactgccctgtcccacctgcctctgtctttcttaaaacagctaaatgcaacaatagcaggaattagcttgtttttgaggttggcaatgaccagttcaaggtgactcttattttcttaagcagtgcttgcaggacgtaaatgtgatgacacttgccctcctttctttatcgcctgggacagactttacaaacagacctgggagaagtcccctaaggggctgcatttatccccatctccctaggggtgatcagcattgtgacagctgggcagagcagtggtgaactgcacccatgtccctgctcacatctcctaagatctcagaattgcctgaggttctagcgtgggctccttctctccagatgatgccatccccacccccctcatttccacacagcatctgaggcatcctgcactaaaagatatatgtacctcgg

SEQ ID NO: 101 - DNA, 473 bp, Homo sapiens
gttagaaatcaataacagaagtaaaactagaaaattcacaatgttttggaaattaaacaccttcttaatcaatggatcaaagaagaaatcacaaggaatgttagaaaatccttaaagatgaatgaaaatgaaaatacaacccaccaaaacctatgtgaccagtgaaagcaatggccaaagggaacattaaaaagcaagaacgatctcaaatcaacaacctaattttacaagttaaggaactggaaaaataagaacaaattaaacccaaagatagcagaagaaagagaataaaaagattaaagatagacaaaatatatactagagacaatagagaaaattaatgaaaacaaaagttaactctttgaaaagatcaacaaaaatttacaaaactttgctagatggactaataacaaaagagaagactcaaaatactaaaaaaaaaaaaaaaaaaaaaaaaaaaaaaaaaccccccc

SEQ ID NO: 102 - DNA, 448 bp, Homo sapiens
gaacagcctctgaggtaagaggaatctcagaaaaggtcaatgtttctgaagccaagagaagagaacttctcaagaaaaaaagagttagtagctgtctagagttccaataaaatcattgagagaagaattgaaggccaggtgcagtggcttacgcctgtaatcccagcactttgggaggccgagttgggcggatcatgaggtcaggagttcgggaccatcctgaccaacatggtgaaaccccgtctctactaaaaatacaaaaattagccaggcatggtggcgcacacctgtaatcccagctactcaggaggctgaggcaggagaattgcttgaacccgggaggcggaggttgtagtgagctgagatcgtgccactgcactccagcctgggcaacagcaagactgcatctcaaaaaaaaaaatcattcaaatgcccgcgtacctgcccg

SEQ ID NO: 103 - DNA, 519 bp, Homo sapiens
ttccaaaaagggaccccccccccgggcccgggggggggggggggggcccccccccgcccccccccacccccccccccccggggggaaaagggccccccccccccccccccccggaaagggggggaccccggggcccccccccccccccgggaaaattttgggatttttaaaaaaaaaaggggttccccccgggaccccgggggggccgggccccccccccctgggaaccccccccccccccccccccaaagggggggaaaaaaggggaaaacccccccccccccccccccccccaaaacttttttttttcctttaaaaaagaacccccccccccccccccggggaaaagggggggggggaaaccccccccccccccaaccccccccccccgggaaaaaacaaccccccccccccccccccccaaaaaggggggacaacggggggggccccccccccccggaaaattttttgttttttttaaaaaaaaagggggttccccccccccccgccccccccccc

SEQ ID NO: 104 - DNA, 326 bp, Homo sapiens
tgtcactgggcaggcggtgcctctatactggtgatgctagaggtgatgtttttggtaaacaggcggggtaagatttgccgagttccttttactttttttaacctttccttatgagcatgcctgtgttgggttgacagtgagggtaataatgacttgttggttgattgtagatattgggctgttaattgtcagttcagtgttttaatctgacgcaggcttatgcggaggagaatgttttcatgttacttatactaacattagttcttctatagggtgatagattggtccaattgggtgtggggagttcagttcccgcgtacctcggc

SEQ ID NO: 105 - DNA, 273 bp, Homo sapiens
ttccgacggcatctacggctcacattttttgtagccacaggcttccacggacttcacgtcattattggctcaactttcctcactatctgcttcatccgccaactaatatttcactttacatccaaacatcactttggcttcgaagccgccgcctgatactggcattttgtagatgtggtttgactatttctgtatgtctccatctattgatgagggtcataaaaaaaaaaaaaaaaaaaaaaaaaaaaaccccccccccccccccccaaaaaa

SEQ ID NO: 106 - DNA, 693 bp, Homo sapiens
gagcccaagtgttccaagtgctgaagccccagcgcctagagcctgtgctctaggagcttctagagaagccaccacaataagaagcccatacacaacaaagagtagatcccttgccacaagtagacagagcttgcaaaaggcaaagaaaatccagcacaaccaaaagtaaataaaaataaaataaaattttttaaaagataccatgttggatggcaactatgtcctagacactacattaggaatcaaagaaataaagatcaagatggataaaaaaagtctctcttaactggatgatctcatagtctaatgaggaagactgatatgatacataaagagataattataacacaaagtaataaacttcagggtagaaaaataaagaaagaacttcagaagcacagtatatttaagagaaagttccgaaattcatgttgattttctaaaagaaataaacaactataactttgtaaacacattttaaaaattttgctttaaactaagaaggaccaaatacctgattatcatagaaatgtcaaattggagatatgtttgcatgactgcacttcccttaaaagaataacatagtgaatttgaaggaccaggagaaataatggagaacataatatatttgtaaccaaaaacttatatactgccactctctgattacttgttaataaaattcaatggcttaaa

SEQ ID NO: 107 - DNA, 275 bp, Homo sapiens
atttccgacggcatctacggctcacattttttgtagccacaggcttccacggacttcacgtcattattggctcaactttcctcactatctgcttcatccgccaactaatatttcactttacatccaaacatcactttggcttcgaagccgccgcctgatactggcattttgtagatgcggtttgactatttctgtatgtctccatctattgatgagggtcttaagaaaaaaaaaaaaaaaaaaaaaaaaaacccgcccgggggggcccccaaaaa

SEQ ID NO: 108 - DNA, 475 bp, Homo sapiens
aggggaagggggcccttttttcccaaaaaaaaccccccggttaaaaaaccccccgtttgtccaaaaccccccccgggatttgggaaacaccaaacctttttttttgtttttgggggaaaaaaaaaaaaaaaaatttgggggggggcccgggctgggggaaattttgggaatttaaaaaaaaaaaaattggggggttggggggaaaaaaagggccagggaccggggtttggtccccggttgtctccggggaaaacccggggggggggaatttaaaaaattttccaaaaaaaaccccaggggggacctggggctcctttaaacccgggggttgggggcttgggggccccctggggggggccccaaaggaaacccccccccccccgggtttttgggtgggctccccccccccggaaaaggggttaaaaccccaaaccccgcgaaaccttttgggaaaaaagggggaaccccttttttt

SEQ ID NO: 109 - DNA, 473 bp, Homo sapiens
tttttgggggttcaattcccttctttttttttaaaatttttataaaaacggtttttcaaaagggggggggggggggggggcccccaaaaaccccccccggttttaggggggttttttcaaaaaaaaaaatttttccttcaaaaaaaaagctttccaaaaaaaaaaaaattaaaaaaattccgggtttaaaaaaaaaaaaaaaccccaaaaaaaaaaaatttttttttggggtttatccccggggaaacccaaaaaaacccggggctttccaaaaaaaccaaaaaaatttttgggaaaaaaaattaaattttccccaaaaaaaaaaaaaaaaaaaaggttttttggcaaatttttgcccaggggggaccccaaaaaaaagaaaaaaaaaaaaaaaaacccccccaaaaaggaaaaaaacccccctttttaaaaaaaaaaaggaaaaggggcaaaaaaaaaaacccccccccccc

SEQ ID NO: 110 - protein, 25 aa, Homo sapiens
GAYPLSIEPIGVRALYLQYTDETFR

SEQ ID NO: 111 - protein, 29 aa, Homo sapiens
SYELPDGQVITIGNERQEYDESGPSIVHR

SEQ ID NO: 112 - protein, 9 aa, Homo sapiens
LEPYADQLR